SEQUENCE LISTING (84 sequences)

SEQ ID NO: 1 - DNA, 1320 bp, Artificial Sequence
  Methyl Transferase From Escherichia coli
atgatgtccggcgaacatactctgaaggcagtacgcgggtcattcatcgatgttacccgtactattgataatccagaagaaatagcctcagctctgcggttcatcgaggatggattgttattaataaagcagggtaaagttgagtggttcggcgagtgggaaaacggtaagcaccagattccggatactattcgtgttcgtgactatcgggggaaacttatagttcccgggttcgtggatacgcacatccattacccccagtccgaaatggtaggagcgtatggcgaacaattattggaatggctcaacaagcatacattcccgactgagcgtagatatgaagatctggaatatgcccgcgaaatgagtgcgtttttcataaagcagcttttgcggaatggcactacaaccgctcttgtctttggtaccgtgcatcctcagtctgtcgatgctctgtttgaggccgcttcacacatcaatatgcggatgatagcaggaaaggtaatgatggatagaaacgcgcccgattacctcctcgatactgccgaatcaagctatcatcaaagcaaggagctgatcgagcgttggcataaaaatggacgccttctttatgcgattacccctagattcgcaccaacgagctcacctgaacagatggccatggcacagcgcttaaaggaagagtatcctgatacatgggtccacacgcacctttgtgagaataaggatgaaattgcatgggtcaaatcactgtacccagatcatgatggctacttagatgtgtatcaccaatatgggttaactggtaagaattgcgtattcgctcactgtgtgcatcttgaagaaaaggaatgggatcgtctgtcagagactaaatcgtcaattgcattctgtcccacatccaatctgtatctcggtagcggcctgtttaatctgaaaaaggcgtggcaaaagaaggtcaaagtcgggatgggtacagacataggcgcaggtacaacgtttaatatgttgcagacgttgaacgaggcctacaaggtcttgcaattgcaaggctatcggctttcagcttacgaagctttttatctggcgactttgggcggagcaaaatccctgggtttagatgatctgatagggaacttcctgcctggtaaagaggctgacttcgtagttatggaacccactgccactccacttcaacaacttcgttatgacaattcggttagtttggtagacaaacttttcgttatgatgacgttaggggacgaccgcagtatctatagaacctacgttgatggtagactggtatatgagcggaattaa

SEQ ID NO: 2 - protein, 439 aa, Artificial Sequence
  Methyl Transferase From Escherichia coli
MMSGEHTLKAVRGSFIDVTRTIDNPEEIASALRFIEDGLLLIKQGKVEWFGEWENGKHQIPDTIRVRDYRGKLIVPGFVDTHIHYPQSEMVGAYGEQLLEWLNKHTFPTERRYEDLEYAREMSAFFIKQLLRNGTTTALVFGTVHPQSVDALFEAASHINMRMIAGKVMMDRNAPDYLLDTAESSYHQSKELIERWHKNGRLLYAITPRFAPTSSPEQMAMAQRLKEEYPDTWVHTHLCENKDEIAWVKSLYPDHDGYLDVYHQYGLTGKNCVFAHCVHLEEKEWDRLSETKSSIAFCPTSNLYLGSGLFNLKKAWQKKVKVGMGTDIGAGTTFNMLQTLNEAYKVLQLQGYRLSAYEAFYLATLGGAKSLGLDDLIGNFLPGKEADFVVMEPTATPLQQLRYDNSVSLVDKLFVMMTLGDDRSIYRTYVDGRLVYERN

SEQ ID NO: 3 - DNA, 471 bp, Artificial Sequence
  Methyl Transferase From Bacillus subtilis
atgaatcacgaaacattcctcaaaagagccgtcacgcttgcttgcgagggtgttaatgccggcattgggggaccatttggcgcagtcatcgttaaagatggggcgatcatagctgagggccagaacaacgtgactacctctaatgacccaacagcacacgcggaggtgacagctatacggaaggcttgcaaagtactcggtgcgtaccaactggacgactgtattttatatacttcctgtgagccatgcccaatgtgcctcggtgcgatatactgggctagaccaaaagctgtcttttacgccgccgaacataccgatgcagcggaagcggggttcgatgactccttcatatacaaggaaatcgacaaaccggccgaggagcgcacaattcccttctaccaggtcacgctgaccgagcacctttctcctttccaagcctggagaaacttcgctaacaagaaagagtattaa

SEQ ID NO: 4 - protein, 156 aa, Artificial Sequence
  Methyl Transferase From Bacillus subtilis
MNHETFLKRAVTLACEGVNAGIGGPFGAVIVKDGAIIAEGQNNVTTSNDPTAHAEVTAIRKACKVLGAYQLDDCILYTSCEPCPMCLGAIYWARPKAVFYAAEHTDAAEAGFDDSFIYKEIDKPAEERTIPFYQVTLTEHLSPFQAWRNFANKKEY

SEQ ID NO: 5 - DNA, 1311 bp, Artificial Sequence
  Methyl Transferase From Klebsiella pneumonia
atgatggactaccagaccgcggtgcgcggtgcgtttttcgatattgccggcgtagcagaaacccccgatgaagtggcagcacaggcccgttacctcgatgatgggttactttttctgcgcgatggaaagataattgcgttgctgccgtggcaagaaggggaagcattcttacatccgctgaagggctatgtagatcagcgcggcaagctgttactgcctggatttgtcgatacacatatccactaccctcagaccgaaatgataggtgcctttggtgagcagctccttgaatggcttacaacatatacgtttcctgtcgaatcacagtttgccgatgccgattatgcacaggaaatcgcgcagttctttgtcaaccagttaatttcacacggcactacaaccgcactggtcttttgcacactgcacccggcctctgccgaggccttattctcggaagcattacggttgaacatgagactgatagccggtaaggtcatgatggatcgccatgtcccagattacttgtgcgaaacggccggtgaatcatacgaacaaacaagagagttgatacttcggtggcatcagcgtggacggttaggttacgccatcacacctcgctttgcgccaacttcaactcctgcccttttagaagcggtccagagacttcgtaccgaatttcctgatacctggttgcagacccacttatcagaaaaccgcgaagaaattgcttgggtgaaacagctgtggccagagcatgagcattatttggacgtctatcaccattatcagctgacaggggagcggtcagtcttcgcgcacggcattcatttggatgatgcggagtggcagtgtctgcacgacactgggtctgccgtcgcattttgtccgacatcaaatctgttcctgggatctggtctgtttcgcttacccgcgtgttggcagcatcaggtaagaatgggaataggtagtgacgttggtgctggaactacattctctatgctccgtacactgggtgaagcgtataaagtcggacagctgcagtcctaccggttgcgtgcatcggaagccttctaccatgctacgttgggaggggctagagcattaagattggaggataaaattggtaacttccagccgggtaaagaggcggattttgtcgtaattgatcccgctgttacacctcttcaacgtctgcgtaccggacgctgccatgacatctatgaacagcttttcgttttaatgacactgggggacgagagaaacatcagtgaaacatgggtcaacggggagcgcgtttggtgccaggattaa

SEQ ID NO: 6 - protein, 436 aa, Artificial Sequence
  Methyl Transferase From Klebsiella pneumoniae
MMDYQTAVRGAFFDIAGVAETPDEVAAQARYLDDGLLFLRDGKIIALLPWQEGEAFLHPLKGYVDQRGKLLLPGFVDTHIHYPQTEMIGAFGEQLLEWLTTYTFPVESQFADADYAQEIAQFFVNQLISHGTTTALVFCTLHPASAEALFSEALRLNMRLIAGKVMMDRHVPDYLCETAGESYEQTRELILRWHQRGRLGYAITPRFAPTSTPALLEAVQRLRTEFPDTWLQTHLSENREEIAWVKQLWPEHEHYLDVYHHYQLTGERSVFAHGIHLDDAEWQCLHDTGSAVAFCPTSNLFLGSGLFRLPACWQHQVRMGIGSDVGAGTTFSMLRTLGEAYKVGQLQSYRLRASEAFYHATLGGARALRLEDKIGNFQPGKEADFVVIDPAVTPLQRLRTGRCHDIYEQLFVLMTLGDERNISETWVNGERVWCQD

SEQ ID NO: 7 - DNA, 1311 bp, Artificial Sequence
  Methyl Transferase From Pseudomonas putida
atgactgtcactcgcaaggcttatcgcgcggcaatacttcactcaatcgctgatcccgcagaggtcggattagacgcgtcacatgagtacttcgaggacggccttcttgtgatcgacggaggtcgtattcaagcggttggccatgcgagtgacctgctgcccaccctggatgctgatatcccggttgaacattatcaggatgctctcattacgcctggctttatagatactcatatccacttcccccaaacgggtatgataggcagttatggggaacagttgctggattggctgaacacctacacatttccgtgcgagaaacagtttgcggataaagatcatgcagatcaagtggcccatgtgttcttaaaagagctgttacgcaacggcaccacgaccgcgctggtatttggttcggtacatcccgaaagcgtaaatgcgctgtttgaggcagctgagcgtcttgatcttcggctcatagctggtaaagttatgatggatcgcaacgcacccgattatctgaccgacactgctgagagcagctacgcccagtccaaagctctcatcgagcggtggcacggtaaaggcagattacactacgccgtgacgccgagatttgcgccaacatctacgccagaacagctcgcacgtgcaggtcaactgctgaaagagcaccctggagtgtatttgcatacccatctcagcgagaacctccaagaaatcgactgggttaagtctttattccctgaacaaaagggctaccttgatgtctatgaccacttcgaactgttaggggaacgcagcgtatttgctcacggggttcatctctgcgatgaagaatgtcagcgtttggcggaaacaggttcggctgtggcattctgccctacgtccaatttatttctcggaagtggactgtttaatcttccccaggccgagagattcaaagtgaacgtggggctggggacagatgtgggtgctggtactagtttctccctgctgaataccctgaacgaagcttataaggttatgcagctgcaaggtgcgcggctccacccgtacaaatctctttacctcgctacacttggtggtgcaagagcgttaagacttgatgaccgcattgggagcctgcgccctggtaatgatgcagactttgtggtgctcgattataaagctactcccttactggactatcggttacagcaatctagatccatcgaggaaacattatttgtgctgacaactctcggggatgatagaactgttcgcgaaacgtatgcggcaggtcggtgtgtccatcaaagagaaggataa

SEQ ID NO: 8 - protein, 436 aa, Artificial Sequence
  Methyl Transferase From Pseudomonas putida
MTVTRKAYRAAILHSIADPAEVGLDASHEYFEDGLLVIDGGRIQAVGHASDLLPTLDADIPVEHYQDALITPGFIDTHIHFPQTGMIGSYGEQLLDWLNTYTFPCEKQFADKDHADQVAHVFLKELLRNGTTTALVFGSVHPESVNALFEAAERLDLRLIAGKVMMDRNAPDYLTDTAESSYAQSKALIERWHGKGRLHYAVTPRFAPTSTPEQLARAGQLLKEHPGVYLHTHLSENLQEIDWVKSLFPEQKGYLDVYDHFELLGERSVFAHGVHLCDEECQRLAETGSAVAFCPTSNLFLGSGLFNLPQAERFKVNVGLGTDVGAGTSFSLLNTLNEAYKVMQLQGARLHPYKSLYLATLGGARALRLDDRIGSLRPGNDADFVVLDYKATPLLDYRLQQSRSIEETLFVLTTLGDDRTVRETYAAGRCVHQREG

SEQ ID NO: 9 - DNA, 1470 bp, Artificial Sequence
  Methyl Transferase From Saccharomyces cerevisiae
atgaccaaatcggatttattgttcgataaatttaacgataaacacggaaaatttttggtattctttggaacattcgtggacacgccgaagctgggagaactccggattcgcgaaaagacgtcagtgggagttttaaacggaattatacggttcgttaaccggaactcgttagatccggttaaggattgcctggatcatgattcttcgttgtctccggaggacgtcaccgtcgtcgatataatcggtaaagataagactcggaacaactccttctactttcccggatttgtcgacactcataatcatgtttcacagtacccaaatgtgggggtttttggtaattcaacactgttggattggctggagaaatatactttcccgatcgaggctgctttggccaacgaaaacattgcccgtgaggtctacaacaaggttatctccaaaactctgagtcatggtacaactactgtggcgtattacaacaccatagatctgaagtctacaaagcttctggcccagttatcatcccttctcggtcagcgtgtacttgttggcaaagtctgtatggataccaatggaccggaatattatattgaagatacgaaaacgagcttcgaatctacggtcaaagttgtcaaatacatacgcgaaacgatttgcgatcctctggtaaacccaatagtgactcctagattcgctccatcgtgcagccgtgagttaatgcagcagttaagcaagcttgttaaggacgaaaatatccatgttcagacgcacctgtcagagaacaaagaagaaatccaatgggtgcaggatctgttccccgagtgcgaatcttatacagacgtctatgataagtatggcctgctcactgagaaaacggttttagcacattgcattcatctgacagatgcagaagcacgtgttataaagcagcggcgctgtggcatcagtcactgcccgataagtaactcctcattgacatcgggagaatgtcgggttcgctggctgctggatcaaggtattaaagttggtctgggaacggatgtctcggcagggcattcatgttcaattttgacaactggacggcaggcgttcgcggttagccgccatttagccatgcgtgagactgatcacgcaaaattatcggtttcagagtgcttgtttcttgcaactatgggcggtgcgcaggttttaagaatggatgagacgttgggaacatttgatgtcggtaaacaattcgatgcgcaaatgatagatacgaatgctccgggctccaacgtagatatgtttcactggcagttgaaggaaaaagatcaaatgcaggagcaagagcaggaacaaggacaggacccttacaaaaatccgccgttactgacaaacgaagatatcattgcgaagtggttctttaatggtgacgatcgtaacaccacaaaagtgtgggtggcaggtcagcaagtttatcagatctaa

SEQ ID NO: 10 - protein, 489 aa, Artificial Sequence
  Methyl Transferase From Saccharomyces cerevisiae
MTKSDLLFDKFNDKHGKFLVFFGTFVDTPKLGELRIREKTSVGVLNGIIRFVNRNSLDPVKDCLDHDSSLSPEDVTVVDIIGKDKTRNNSFYFPGFVDTHNHVSQYPNVGVFGNSTLLDWLEKYTFPIEAALANENIAREVYNKVISKTLSHGTTTVAYYNTIDLKSTKLLAQLSSLLGQRVLVGKVCMDTNGPEYYIEDTKTSFESTVKVVKYIRETICDPLVNPIVTPRFAPSCSRELMQQLSKLVKDENIHVQTHLSENKEEIQWVQDLFPECESYTDVYDKYGLLTEKTVLAHCIHLTDAEARVIKQRRCGISHCPISNSSLTSGECRVRWLLDQGIKVGLGTDVSAGHSCSILTTGRQAFAVSRHLAMRETDHAKLSVSECLFLATMGGAQVLRMDETLGTFDVGKQFDAQMIDTNAPGSNVDMFHWQLKEKDQMQEQEQEQGQDPYKNPPLLTNEDIIAKWFFNGDDRNTTKVWVAGQQVYQI

SEQ ID NO: 11 - DNA, 1281 bp, Artificial Sequence
  Methyl Transferase From Clostridium sp.
atgcgggatttggagaagaacataaaaattctgaagggcaacatcatctacaccgaaaactctgattcatttaacgttgtggaaaaggggtttatcattgtaaaggatggcattatcgatggcacgtacgagaaattacctgaagaatttaaagatgttcaagtcgaggattacggtgataaactgattatacctgggtttatcgaccttcacacccatgcatctcagttcgctatcaagggtattggatatgataaagagcttttgccatggttggaaacttacacatttcctgaggaggcgaagtttatcgacaaggtgtatgccgaaaaagtatataaggagtttgtggacgaattgtatgaggagggcactacccgtgcagtaatttttgctacaatacatagcgaatctacagagatcctgatgaaattactggaagaaaaaggcattgaggcttatgttgggaaagtcaatatggatcgcaattgccctgaatccctcaaagaaaattcagacgagtctgtggaggcgaccatcaagtggattgagaactcaagcaagaaatataagtatgttaaaccaatcatcaccccaagattcgtaccttcatgtacaggccacttaatgaaagaactgggcagcatcgcagtttcgaaatcgatgccggtccagagccatttgtccgagaaccttagtgaaatagagtgggttaaagaattgcaccccgaatgtaagaactacggcgaagtgtatgacaagttcaacttgtttggccaaaccaaaacaattatggcgcactgcgtgtatttaacagaggatgagatcaacgctatagaagataataacgtgactattgctcactgcccaaccagcaacgttaatatctcgtcgggaattgctccgatcaatacgttgctgagaaagaagaaagtgaaaattggactcggctccgatattgccggaggggaaagtctgagtcttctttctgtgatgaaaagtgcggtttctttatcgaaaatgcgcgcggtgtgtttcaaagaagaagaaaaggcgctgacgctccccgaagtgttttacttagcgactaaaggtggcggttccttctttggtaatgtaggctcgtttgagaaatcttacgagtttgatgccctgattatcgacgacgactctttatggaaagttaataaaggaaatattgaggaacgtctggagaaactggtgtacttaggagacaaacgcaatattataaaacgctatgtctgtggacgcgaaatctaa

SEQ ID NO: 12 - protein, 426 aa, Artificial Sequence
  Methyl Transferase From Clostridium sp.
MRDLEKNIKILKGNIIYTENSDSFNVVEKGFIIVKDGIIDGTYEKLPEEFKDVQVEDYGDKLIIPGFIDLHTHASQFAIKGIGYDKELLPWLETYTFPEEAKFIDKVYAEKVYKEFVDELYEEGTTRAVIFATIHSESTEILMKLLEEKGIEAYVGKVNMDRNCPESLKENSDESVEATIKWIENSSKKYKYVKPIITPRFVPSCTGHLMKELGSIAVSKSMPVQSHLSENLSEIEWVKELHPECKNYGEVYDKFNLFGQTKTIMAHCVYLTEDEINAIEDNNVTIAHCPTSNVNISSGIAPINTLLRKKKVKIGLGSDIAGGESLSLLSVMKSAVSLSKMRAVCFKEEEKALTLPEVFYLATKGGGSFFGNVGSFEKSYEFDALIIDDDSLWKVNKGNIEERLEKLVYLGDKRNIIKRYVCGREI

SEQ ID NO: 13 - DNA, 483 bp, Artificial Sequence
  Methyl Transferase From Arthrobacter aurescens
atgtcagctcacagtgcacaacagcgggatgagtatcttaaccttgccatacagctggcggtgcacaatgtcagcgacggcggtggaccttttggggcagtagtagtgactgctgacggaacagtgcatgagggagtcaaccgggtgacacgcgatcatgacccaacggcacatgctgaagtggtcgccatacgtcgcgcagctgcagcgtcgaaacggttcgacctgactgggtctgtcttatatgcgtcgtgcgaaccgtgtcccctgtgcctgtctgctacgttgtgggctcgcattgggcatgtctacttcgcagcggatagacatggagcagcaaaggccgggttcgacgatgcggtgttttatgaatatttcgcaggcacacggcccgagttacttcccgttgaacatgcagagcttgctgccagtaacgaaccatttgacgcatggagaaaccacgctcgccgtacagcctattaa

SEQ ID NO: 14 - protein, 160 aa, Artificial Sequence
  Methyl Transferase From Arthrobacter aurescens
MSAHSAQQRDEYLNLAIQLAVHNVSDGGGPFGAVVVTADGTVHEGVNRVTRDHDPTAHAEVVAIRRAAAASKRFDLTGSVLYASCEPCPLCLSATLWARIGHVYFAADRHGAAKAGFDDAVFYEYFAGTRPELLPVEHAELAASNEPFDAWRNHARRTAY

SEQ ID NO: 15 - DNA, 1398 bp, Artificial Sequence
  Methyl Transferase From Bradyrhizobium japonicum
atgacgactgtaggcatccggggcactttctttgactttgtggatgatccatggaagcacataggtaacgagcaagcggcagcgagattccaccaggacgggctcatggtagtcacaggtggcgtgataaaagcttttgggccacacgacaaaatcgctgcggcgcatcccggggttgagattacacacatcaaggaccggataatcgttccgggttttattgatggtcacattcatctgccgcaaactcgtgtccttggtgcgtatggtgagcagttgcttccgtggctgcagaaaagtatctaccccgaagagatcaaatacaaagatcgtaactacgctcgcgagggcgtgaaacgtttcttagatgcgctgctggcagctggtactacgacttgtcaggcttttacgagctcctctcctgttgcgactgaagagctttttgaagaagcagcacggcggaatatgcgtgttatcgcgggtctgacgggcatcgatcgcaatgctcctgctgaattcatagatactccagaaaacttctaccgcgatagtaaacggctcattgcgcagtatcataacaaagggagaaacttatacgcgatcacaccccgtttcgcatttggagcgagccccgaactgctcaaagcatgccaacgtttaaaacacgagcatcctgattgctgggtcaatacccatatttctgagaatcccgctgagtgctcaggggttctggttgagcatccggattgtcaggactacctgggtgtttacgagaagtttgatctcgtaggaccaaaatttagcggaggacacggggtatatctgtcgaataacgagttccatagaatgtctaagaagggtgcggctgtggtattttgcccttgctccaatttgtttcttggaagcggtttgtttcggctcggtagagccaccgatccagaacatcgggtcaaaatgagtttcgggaccgatgtgggcggcggtaatcgcttttctatgatttccgttttggacgacgcctataaagtaggtatgtgcaacaacactatgctggatggttcgatagatcccgcacggaaagatctggctgaggcagagcgtaataaattatccccataccgcggtttttggtcagttaccttagggggtgcagaaggcctttacattgacgataaattaggcaattttgaaccaggtaaagaagcagattttgtagcgttagatccgaacggtggacagcttgctcagccatggcaccaaagtcttatagctgacggagcgggaccgcgtaccgtagatgacgcagccaacatgctgttctccgtaatgatggttggggatgatcgttgtgtggacgagacgtgggtcatgggaaaacggctgtacaaacagtcataa

SEQ ID NO: 16 - protein, 465 aa, Artificial Sequence
  Methyl Transferase From Bradyrhizobium japonicum
MTTVGIRGTFFDFVDDPWKHIGNEQAAARFHQDGLMVVTGGVIKAFGPHDKIAAAHPGVEITHIKDRIIVPGFIDGHIHLPQTRVLGAYGEQLLPWLQKSIYPEEIKYKDRNYAREGVKRFLDALLAAGTTTCQAFTSSSPVATEELFEEAARRNMRVIAGLTGIDRNAPAEFIDTPENFYRDSKRLIAQYHNKGRNLYAITPRFAFGASPELLKACQRLKHEHPDCWVNTHISENPAECSGVLVEHPDCQDYLGVYEKFDLVGPKFSGGHGVYLSNNEFHRMSKKGAAVVFCPCSNLFLGSGLFRLGRATDPEHRVKMSFGTDVGGGNRFSMISVLDDAYKVGMCNNTMLDGSIDPARKDLAEAERNKLSPYRGFWSVTLGGAEGLYIDDKLGNFEPGKEADFVALDPNGGQLAQPWHQSLIADGAGPRTVDDAANMLFSVMMVGDDRCVDETWVMGKRLYKQS

SEQ ID NO: 17 - DNA, 1320 bp, Artificial Sequence
  Methyl Transferase From Deinococcus radiodurans
atgaagatttaccgcagtacccttctgcatacaccagccagcccgtttgcggtgccggatgcgctgcagactttcagcgacggtgctctggccgtaggtgatacgggtaccattgcacaccttggtacattcaccgaagtacttgcagaagtgcgtgccgcatgcccagatgcggaagtacacgatctgcgtggtggcgttctgctgcctggctttatcgatactcacgttcactatccgcaggtacgcgttttgggagggctggggatggccttattagagtggcttgatcgtaatacactgcccgaagaagctcgcttagctgatgcggcttatgctcgcacaatagccggcgagttccttcacggtcttgcaagtcatggaactactaccgccctggtgtttggtagtcactttgcgggagctatggatgagttctttgcggaagctgcggcaagaggtttacgggtggttgcgggccaagtggtcagtgatcgtctgctgcgccccgaattacacactactccggagcgggcttatgccgagggcaaagcgctgattgaacgttggcatggacaaggaagatctctgtatgccgtgactccgcgcttttcgctctcggccagtgagggcatcctcgatgcgtgtgcggcattgctgactgaatttccagacgtgcggtttacgagtcacataaacgagaataatcaggagatcgaagtggtgcgggggctgttcccaggtgcacgtgactacctcgatacttatgaaagagctggcttggtgacgccgcgctctgtttttgcccataacgtgcaccccaacgagcgggaactcggtgtcttggcggcacaacggtgctctgtcgcgcattgcccatgtagtaattcagccttagggtctggacttttcccactgcgtcggcacctggcagcaggcgtgcatgtagcgctggggacggacgtgggtggaggaacgggattcagcttactcaaagaaggtttgcaagcgtatttcatgcagcaactgttgggtgaagaaggtgcggcactgtctcctgcccatctcttatacttagcaacactggcgggcgcgcaggcacttggtctggatggacaagttggagatttcacgcctggaaaacagttcgacgctgtctggttgcgtccaagagcagggtcaaccctggcaaccgtcctcgctcatgctgagtctgaagaacgtacgctcgccgcgttgttcgcactcggtacaggggatgatgtcgaacgtgtgtgggttggcggaggcgtggtctttgcccgtgaagcagaagcgtaa

SEQ ID NO: 18 - protein, 439 aa, Artificial Sequence
  Methyl Transferase From Deinococcus radiodurans
MKIYRSTLLHTPASPFAVPDALQTFSDGALAVGDTGTIAHLGTFTEVLAEVRAACPDAEVHDLRGGVLLPGFIDTHVHYPQVRVLGGLGMALLEWLDRNTLPEEARLADAAYARTIAGEFLHGLASHGTTTALVFGSHFAGAMDEFFAEAAARGLRVVAGQVVSDRLLRPELHTTPERAYAEGKALIERWHGQGRSLYAVTPRFSLSASEGILDACAALLTEFPDVRFTSHINENNQEIEVVRGLFPGARDYLDTYERAGLVTPRSVFAHNVHPNERELGVLAAQRCSVAHCPCSNSALGSGLFPLRRHLAAGVHVALGTDVGGGTGFSLLKEGLQAYFMQQLLGEEGAALSPAHLLYLATLAGAQALGLDGQVGDFTPGKQFDAVWLRPRAGSTLATVLAHAESEERTLAALFALGTGDDVERVWVGGGVVFAREAEA

SEQ ID NO: 19 - DNA, 1320 bp, Artificial Sequence
  Methyl Transferase From Escherichia coli
atgatgtcaggagaacacacgttaaaagcggtacgaggcagttttattgatgtcacccgtacgatcgataacccggaagagattgcctctgcgctgcggtttattgaggatggtttattactcattaaacagggaaaagtggaatggtttggcgaatgggaaaacggaaagcatcaaattcctgacaccattcgcgtgcgcgactatcgcggcaaactgatagtaccgggctttgtcgatacacatatccattatccgcaaagtgaaatggtgggggcctatggtgagcaattgctggagtggttgaataaacacaccttccctactgaacgtcgttatgaggatttagagtacgcccgcgaaatgtcggcgttcttcatcaagcagcttttacgtaacggaaccaccacggcgctggtgtttggcactgttcatccgcaatctgttgatgcgctgtttgaagccgccagtcatatcaatatgcgtatgattgccggtaaggtgatgatggaccgcaacgcaccggattatctgctcgacactgccgaaagcagctatcaccaaagcaaagaactgatcgaacgctggcacaaaaatggtcgtctgctatatgcgattacgccacgcttcgccccgacctcatctcctgaacagatggcgatggcgcaacgcctgaaagaagaatatccggatacgtgggtacatacccatctctgtgaaaacaaagatgaaattgcctgggtgaaatcgctttatcctgaccatgatggttatctggatgtttaccatcagtacggcctgaccggtaaaaactgtgtctttgctcactgcgtccatctcgaagaaaaagagtgggatcgtctcagcgaaaccaaatccagcattgctttctgtccgacctccaacctttacctcggcagcggcttattcaacttgaaaaaagcatggcagaagaaagttaaagtgggcatgggaacggatatcggtgccggaaccactttcaacatgctgcaaacgctgaacgaagcctacaaagtattgcaattacaaggctatcgcctctcggcatatgaagcgttttacctggccacgctcggcggagcgaaatctctgggccttgacgatttgattggcaactttttacctggcaaagaggctgatttcgtggtgatggaacccaccgccactccgctacagcagctgcgctatgacaactctgtttctttagtcgacaaattgttcgtgatgatgacgttgggcgatgaccgttcgatctaccgcacctacgttgatggtcgtctggtgtacgaacgcaactaa

SEQ ID NO: 20 - protein, 439 aa, Artificial Sequence; identical to SEQ ID NO: 2
  Methyl Transferase From Escherichia coli

SEQ ID NO: 21 - DNA, 1320 bp, Artificial Sequence
  Methyl Transferase From Escherichia coli
atgatgtcaggagaacacacgttaaaagcggtacgaggcagttttattgatgtcacccgtacggtcgataacccggaagagattgcctctgcgctgcggtttattgaggatggtttattactcattaaacagggaaaagtggaatggtttggcgaatgggaaaacggaaagcatcaaattcctgacaccattcgcgtgcgcgactatcgcggcaaactgatagtaccgggctttgtcgatacacatatccattatccgcaaagtgaaatggtgggggcctatggtgagcaattgctggagtggttgaataaacacaccttccctactgaacggcgttatgaggatttagagtacgcccgcgaaatgtcggcgttcttcatcaagcagcttttacgtaacggaaccaccacggcgctggtgtttggcactgttcatccgcaatccgttgatgcgctgtttgaagccgccagtcatatcaatatgcgtatgattgccggtaaggtgatgatggaccgtaacgcaccggattatctgctcgacactgccgaaagcagctatcaccaaagcaaagaactgattgaacgctggcacaaaaatggtcgtctgctatatgcgattacgccacgcttcgccccgacctcatctcctgaacagatggcgatggcgcaacgcctgaaagaagaatatccggatacgtgggtacatacccatctcagtgaaaacaaagatgaaattgcctgggtgaaatcgctttatcctgaccatgatggttatctggatgtttaccatcagtacggcctgaccggtaaaaactgtgtctttgctcactgcgtccatctcgaagaaaaagagtgggatcgtctcagcgaaaccaaatccagcattgctttctgtccgacctccaacctttacctcggcagcggcttattcaacttgaaaaaagcatggcagaagaaagttaaagtgggcatgggaacggatatcggtgccggaaccactttcaacatgctgcaaacgctgaacgaagcctacaaagtattgcaattacaaggctatcgcctctcggcatatgaagcgttttacttggccacgctcggcggagcgaaatctctgggccttgacgatttgattggcaactttttacctggcaaagaggctgatttcgtggtgatggaacccaccgccactccgctacagcagctgcgctatgacaactctgtttctttagtcgacaaattgttcgtgatgatgacgttgggcgatgaccgttcgatctaccgcacctacgttgatggtcgtctggtgtacgaacgcaactaa

SEQ ID NO: 22 - protein, 439 aa, Artificial Sequence
  Methyl Transferase From Escherichia coli
MMSGEHTLKAVRGSFIDVTRTVDNPEEIASALRFIEDGLLLIKQGKVEWFGEWENGKHQIPDTIRVRDYRGKLIVPGFVDTHIHYPQSEMVGAYGEQLLEWLNKHTFPTERRYEDLEYAREMSAFFIKQLLRNGTTTALVFGTVHPQSVDALFEAASHINMRMIAGKVMMDRNAPDYLLDTAESSYHQSKELIERWHKNGRLLYAITPRFAPTSSPEQMAMAQRLKEEYPDTWVHTHLSENKDEIAWVKSLYPDHDGYLDVYHQYGLTGKNCVFAHCVHLEEKEWDRLSETKSSIAFCPTSNLYLGSGLFNLKKAWQKKVKVGMGTDIGAGTTFNMLQTLNEAYKVLQLQGYRLSAYEAFYLATLGGAKSLGLDDLIGNFLPGKEADFVVMEPTATPLQQLRYDNSVSLVDKLFVMMTLGDDRSIYRTYVDGRLVYERN

SEQ ID NO: 23 - DNA, 1137 bp, Artificial Sequence
  Methyl Transferase From Coffea arabica
atggagcttcaagaggtactgcacatgaacgagggcgaaggagatacgagctacgcgaaaaatgcgagttataacctggcgctcgcaaaagtgaaacctttcctggaacagtgtatacgcgagttgcttcgcgcaaacctgccaaacataaataaatgcattaaggtagcggatttaggctgtgcctcaggcccaaatacgctgctcactgttcgtgacatagtacagtcgatagacaaagttggccaggaggagaaaaacgaattagaacggccaacgatacaaatttttctgaacgacctgttccaaaacgattttaattccgtctttaagctgcttccgtctttctatcggaaactcgagaaggaaaatggtcgtaaaatcggtagttgtctgattagtgcgatgcccggctcgttctatggtagactttttccggaagagtccatgcattttctgcattcttgttacagtgtgcactggctgagtcaggttccttcggggttagtgatcgaacttggtatcggcgcgaacaaaggaagtatctacagctccaaaggatgccgtccaccggtccagaaggcctatttggaccagtttacaaaggacttcaccaccttcctgcgcatacatagtaaagaactgttttctcgtggccgcatgctcttaacttgtatttgcaaggttgatgagtttgatgaacctaaccctctggaccttcttgacatggcaatcaatgaccttattgtcgaaggcttgcttgaggaggagaaattagactcattcaatattccgtttttcacgccatccgccgaagaagtaaagtgtatcgtagaagaagaagggagctgtgagatcctctatctcgagacattcaaggcccactatgatgcagcgttctccatagacgacgattaccctgttcggtcacacgaacaaatcaaagccgaatatgtggccagtctgatcagatctgtctatgaacctatcctcgcctcccactttggcgaagcaataatgcccgacctgtttcaccgccttgctaagcacgctgccaaagttttgcacatggggaaggggtgttacaacaacttgatcatttcacttgccaagaaaccggagaagtctgatgtgtaa

SEQ ID NO: 24 - protein, 378 aa, Artificial Sequence
  Methyl Transferase From Coffea arabica
MELQEVLHMNEGEGDTSYAKNASYNLALAKVKPFLEQCIRELLRANLPNINKCIKVADLGCASGPNTLLTVRDIVQSIDKVGQEEKNELERPTIQIFLNDLFQNDFNSVFKLLPSFYRKLEKENGRKIGSCLISAMPGSFYGRLFPEESMHFLHSCYSVHWLSQVPSGLVIELGIGANKGSIYSSKGCRPPVQKAYLDQFTKDFTTFLRIHSKELFSRGRMLLTCICKVDEFDEPNPLDLLDMAINDLIVEGLLEEEKLDSFNIPFFTPSAEEVKCIVEEEGSCEILYLETFKAHYDAAFSIDDDYPVRSHEQIKAEYVASLIRSVYEPILASHFGEAIMPDLFHRLAKHAAKVLHMGKGCYNNLIISLAKKPEKSDV

SEQ ID NO: 25 - DNA, 1155 bp, Artificial Sequence
  Methyl Transferase From Coffea arabica
atggaactgcaggccgttttacacatgaatggaggggaaggagacacgagctatgctaaaaattcttcatataacctggcgttggcgaaagtaaaaccagtgttagaacaatgcattcgtgaactgttacgggcgaatcttccaaatattaacaattgtataaaagtagcggaccttggctgcgcaagtggaccgaacacgttattaactgttagagatatcgtacaaagtattgacaaggttggccaggaggagaaaaatgaactggagcggccgactatacagatattcctgaatgatttatttcagaacgatttcaatagcgttttcaagctgcttccttcgttttaccgtaaacttgagaaagaaaatggacggaaaattgggtcatgtttaatttccgcaatgcccggcagtttctatgggcgcttgtttccggaagaaagcatgcattttattcactcatgctattcgttccactggctgtcacaagtgccaagtggactcgttattgaactggggatctcagccaacaaaggcagtatatattcctcaaaggcctcaagaccgcctgtgcaaaaagcttacttagaccagttcacaaaggattttacaacgttccttcgcattcacagcaaggaattgttctcacgcggacggatgcttcttacatgtatctgcaaagtggatgagtatgacgaaccaaaccctcttgacttgttagacatggcgataaacgatctcattgtggaaggtcatctcgaggaagaaaagttagcttcttttaacctgccatttttcacaccatcggccgaggaggttaagtgcatcgttgaggaagagggttctttcgagatcctctatctcgagacatttaaagcgcattatgatgcggggtttagcatagatgatgattacccagtccggtcccattttcaagtatatggcgatgaacatattaaagcagagtatgtcgcatctctgatccgctccgtttatgagccgattcttgcaagtcattttggtgaggctatcatgccagacttgtttcaccggttagccaaacacgctgctaaggtactccatctgggtaagggttgttataacaatctcataatttctcttgcgaagaagccagaaaaatcagacatgtaa

SEQ ID NO: 26 - protein, 384 aa, Artificial Sequence
  Methyl Transferase From Coffea arabica
MELQAVLHMNGGEGDTSYAKNSSYNLALAKVKPVLEQCIRELLRANLPNINNCIKVADLGCASGPNTLLTVRDIVQSIDKVGQEEKNELERPTIQIFLNDLFQNDFNSVFKLLPSFYRKLEKENGRKIGSCLISAMPGSFYGRLFPEESMHFIHSCYSFHWLSQVPSGLVIELGISANKGSIYSSKASRPPVQKAYLDQFTKDFTTFLRIHSKELFSRGRMLLTCICKVDEYDEPNPLDLLDMAINDLIVEGHLEEEKLASFNLPFFTPSAEEVKCIVEEEGSFEILYLETFKAHYDAGFSIDDDYPVRSHFQVYGDEHIKAEYVASLIRSVYEPILASHFGEAIMPDLFHRLAKHAAKVLHLGKGCYNNLIISLAKKPEKSDM

SEQ ID NO: 27 - DNA, 1119 bp, Artificial Sequence
  Methyl Transferase From Coffea arabica
atggaattgcaagaagttctgcgtatgaacggtggcgaaggtgacactagctacgctaaaaatagtgcttataaccaactggtactcgcaaaagttaaaccggttttggagcagtgtgttcgtgaactgctgagagcaaacttaccgaacataaataaatgcatcaaagttgccgatttgggctgtgcaagcggccctaatacccttttaaccgttcgggatatcgtgcagtcaatcgataaagtaggtcaagagaaaaagaatgaattagagcgcccaactatccagatctttcttaacgacctgtttcccaacgacttcaatagcgtttttaaactcttgccttctttttatcggaaattagagaaggaaaatgggcgtaagatcggttcctgccttatcggtgcgatgccaggttcattttactcccgtttatttccggaagaaagtatgcattttctccactcttgctattgtcttcagtggttatctcaggtccctagtggattggtaacagaattagggattagcactaacaagggctccatttatagttcgaaagcctcacgtctgcctgtacaaaaggcatatcttgatcagtttacgaaagactttactaccttcctgagaatccattcagaggagttgttttcgcatgggcggatgcttcttacatgtatatgcaaaggagttgaattagacgcaagaaatgctatcgatctgttagagatggcaattaacgacttagtagtcgagggtcatcttgaagaggaaaaacttgactcttttaacctgccggtctatattccttcagccgaggaagttaaatgcattgtagaagaagaggggtcctttgagatactctatcttgagacttttaaggtcttatacgacgcggggttctcaatcgacgatgagcacatcaaagcagaatatgttgcctcttcggtgcgggcggtatatgaaccgatattagccagtcatttcggagaagcgattataccggatatctttcatcgttttgctaagcatgccgctaaagtactccctttggggaaaggattttataacaatctgatcatttcacttgccaagaaacccgagaaatctgatgtgtaa

SEQ ID NO: 28 - protein, 372 aa, Artificial Sequence
  Methyl Transferase From Coffea arabica
MELQEVLRMNGGEGDTSYAKNSAYNQLVLAKVKPVLEQCVRELLRANLPNINKCIKVADLGCASGPNTLLTVRDIVQSIDKVGQEKKNELERPTIQIFLNDLFPNDFNSVFKLLPSFYRKLEKENGRKIGSCLIGAMPGSFYSRLFPEESMHFLHSCYCLQWLSQVPSGLVTELGISTNKGSIYSSKASRLPVQKAYLDQFTKDFTTFLRIHSEELFSHGRMLLTCICKGVELDARNAIDLLEMAINDLVVEGHLEEEKLDSFNLPVYIPSAEEVKCIVEEEGSFEILYLETFKVLYDAGFSIDDEHIKAEYVASSVRAVYEPILASHFGEAIIPDIFHRFAKHAAKVLPLGKGFYNNLIISLAKKPEKSDV

SEQ ID NO: 29 - DNA, 1158 bp, Artificial Sequence
  Methyl Transferase From Coffea arabica
atggaattgcaagaggtgttgcgcatgaacggaggggaaggggatactagttatgcaaagaattccgcctataatcagttggtactggccaaggtcaaacccgtattagagcaatgcgtgcgggagttattacgtgcgaatctgccaaacatcaataaatgtataaaagttgcagacttgggttgtgccagtgggcctaacacacttctgacggtaagagacatcgttcagtccatcgataaagtcggacaagagaagaaaaacgaattagagcgcccgacgatccaaattttccttaatgatctgttccccaacgacttcaactcggtcttcaaacttctgccctcgttttatcgcaagttggaaaaggagaatggtcgcaaaattggttcgtgtttaatcggcgcgatgccgggtagtttttattcgcgcttgtttccagaagaatcaatgcatttcttacactcgtgttactgcttacagtggttgtcacaggtcccttcaggattggtaactgaactggggattagcacaaacaaagggtcaatatattcaagtaaagctagtagactgcccgtgcaaaaggcatatctggatcagttcacaaaagattttactacctttctgcgtatccatagtgaagagctgttctcgcatggtcggatgttattaacctgtatatgtaagggtgttgaactcgatgcacggaatgccatcgacctgcttgaaatggcgattaatgatctggtggtggagggacacctcgaagaagaaaagctggactcgttcaacctcccagtttatataccgagtgctgaagaagttaagtgcatcgtggaagaagagggtagctttgaaatcctgtaccttgaaacgttcaaagtgctgtacgacgcaggattttcaattgatgatgactatcccgtgcgctcacattttcaagtatatggagatgaacacatcaaagcagaatatgtagcgagtttaatccgtagtgtctatgagccaatcttagcaagtcatttcggtgaggcgattatgccagacttgtttcatcgcctggctaaacacgccgctaaagttctccaccttggaaaagggttctataataatctgataatctctcttgccaaaaagcccgaaaaatccgatgtctaa

SEQ ID NO: 30 - protein, 385 aa, Artificial Sequence
  Methyl Transferase From Coffea arabica
MELQEVLRMNGGEGDTSYAKNSAYNQLVLAKVKPVLEQCVRELLRANLPNINKCIKVADLGCASGPNTLLTVRDIVQSIDKVGQEKKNELERPTIQIFLNDLFPNDFNSVFKLLPSFYRKLEKENGRKIGSCLIGAMPGSFYSRLFPEESMHFLHSCYCLQWLSQVPSGLVTELGISTNKGSIYSSKASRLPVQKAYLDQFTKDFTTFLRIHSEELFSHGRMLLTCICKGVELDARNAIDLLEMAINDLVVEGHLEEEKLDSFNLPVYIPSAEEVKCIVEEEGSFEILYLETFKVLYDAGFSIDDDYPVRSHFQVYGDEHIKAEYVASLIRSVYEPILASHFGEAIMPDLFHRLAKHAAKVLHLGKGFYNNLIISLAKKPEKSDV

SEQ ID NO: 31 - DNA, 1161 bp, Artificial Sequence
  Methyl Transferase From Coffea liberica
atggaactccaggaagttcttcggatgaatggtggcgaaggcgacacaagctatgccaaaaattcgtcatataatcttgccctggcgaaagtaaaacccgttttggaacagtgtatccgggaattattacgtgctaacctgcccaacattaataattgcattaaagtagctgatctcggttgcgcatcgggaccgaatacgttacttactgtcagagacatcgttcagtcgattgacaaacttgggctggaggaaaagaatgagctggaacgcccgacaatacagatatttctgaacgatctgtttcagaacgactttaactctgtctttaagctgttacccagtttctaccgcaaattggaaaaagaaaacggccgtaagattggctcctgcctcatttccgctatgccgggttcattctatggtagattgttccccgaggaatcaatgcacttcctgcattcatgttactgcctgcactggttgtcccaggttccctcgggccttgtcacggaactgggtatctcagcaaataagggaagtatatactcttcaaaggccagtcgcctgccggttagaaaagcgtatcttgaccaattcactaaggatttcaccactttcttgcgcatccactcagaggagctgttttctcatggaagaatgctgctcacttgcatttgcaaaggggtagaatttgatgcccggaatgccatagatttattagaaatggctattaatgacctggtggtggagggtcacttggaagaggaaaaattggatagctttaacctgccggtgtatattccgtcaacagaggaggtgaagtgcatcgtagaagaagaaggcagtttcgagattctgtacctggaaactttcaaagttctgtacgatgcaggtttctcaattgacgatgattatccgctccgtagtcatgtccaggtgtattcagatgagcatattaaggcggaatacgttgcgtcatcggtacgggcagtatatgagcctatccttgcatcacactttggtgaagctatcattcctgatatctttcatcggttcgctaaacatgcggcgaaagtgctgcctttaggtaaaggcttttataacaaccttatcatctctctggctaaaaagcctgaaaagtccgacgtgaaccactaa

SEQ ID NO: 32 - protein, 386 aa, Artificial Sequence
  Methyl Transferase From Coffea liberica
MELQEVLRMNGGEGDTSYAKNSSYNLALAKVKPVLEQCIRELLRANLPNINNCIKVADLGCASGPNTLLTVRDIVQSIDKLGLEEKNELERPTIQIFLNDLFQNDFNSVFKLLPSFYRKLEKENGRKIGSCLISAMPGSFYGRLFPEESMHFLHSCYCLHWLSQVPSGLVTELGISANKGSIYSSKASRLPVRKAYLDQFTKDFTTFLRIHSEELFSHGRMLLTCICKGVEFDARNAIDLLEMAINDLVVEGHLEEEKLDSFNLPVYIPSTEEVKCIVEEEGSFEILYLETFKVLYDAGFSIDDDYPLRSHVQVYSDEHIKAEYVASSVRAVYEPILASHFGEAIIPDIFHRFAKHAAKVLPLGKGFYNNLIISLAKKPEKSDVNH

SEQ ID NO: 33 - DNA, 1116 bp, Artificial Sequence
  Methyl Transferase From Coffea canephora
atggaacttcaggaagtccttcacatgaatgagggagagggagatacctcttacgccaagaacgcttcttataacctggcgttggccaaagtgaagcctttccttgaacagtgcattagagaattactgcgggccaatttaccaaacattaataagtacatcaaagtggctgacttaggttgcgcgagcggtccaaacacgctcctgaccgtgcgtgatatcgtacaatcaatagataaagttggacaggaggagaagaatgaactggagcggccgacgatacaaatctttctgaatgatctctttcaaaacgatttcaactcggtgtttaagctgctgccaagtttctatcggaaacttgaaaaggaaaacgggcgtaaaattggaagttgcctgatttcagccatgccaggttctttttatggccgtttattccccgaggaatcaatgcactttcttcacagttgctactcagtccattggctgtcgcaggttccttccggccttgtaatcgaactgggtattggtgcaaataaagggagcatctactcttcaaaagggtgccgtccgcctgtgcaaaaagcttatttggatcaatttacgaaagacttcactacgtttttgcggatacattccgaagagctgttttctcacggacgcatgctcctgacatgcatctgcaaaggtgttgagttggatgcccgcaatgccatcgatttgcttgaaatggcaattaatgacctcgtagtggaaggccatctggaagaggagaagcttgactctttcaacttgcctgtatacataccatctgccgaagaagtaaaatgcatcgtggaggaagaaggttctttcgagatcttgtatcttgaaacatttaaggtcttgtatgatgctggctttagtatagatgatgagcacatcaaggcggagtatgtcgcctcttcagttagagccgtttatgaacctatattggcgagccatttcggagaggccattatacccgatatcttccatcggtttgccaaacatgccgcaaaagttttaccccttggcaaaggtttctacaacaacctgattatttcgctcgcgaagaaacccgaaaaatctgatgtttaa

SEQ ID NO: 34 - protein, 371 aa, Artificial Sequence
  Methyl Transferase From Coffea canephora
MELQEVLHMNEGEGDTSYAKNASYNLALAKVKPFLEQCIRELLRANLPNINKYIKVADLGCASGPNTLLTVRDIVQSIDKVGQEEKNELERPTIQIFLNDLFQNDFNSVFKLLPSFYRKLEKENGRKIGSCLISAMPGSFYGRLFPEESMHFLHSCYSVHWLSQVPSGLVIELGIGANKGSIYSSKGCRPPVQKAYLDQFTKDFTTFLRIHSEELFSHGRMLLTCICKGVELDARNAIDLLEMAINDLVVEGHLEEEKLDSFNLPVYIPSAEEVKCIVEEEGSFEILYLETFKVLYDAGFSIDDEHIKAEYVASSVRAVYEPILASHFGEAIIPDIFHRFAKHAAKVLPLGKGFYNNLIISLAKKPEKSDV

SEQ ID NO: 35 - DNA, 1158 bp, Artificial Sequence
  Methyl Transferase From Coffea arabica
atggaactccaggaggtgctgcagatgaatggcggtgaaggggatacatcctatgccaagaattcggcttataatcagttggtgcttgctaaggtgaaacctgtccttgagcagtgcgtcagagaactccttcgcgctaaccttccgaacattaataaatgcattaaagtcgctgacttgggctgcgcgagtggacccaataccttattaactgttcgcgacatagtccagtccatagacaaagtgggccaggagaagaagaacgaactggaacgcccgactattcaaatttttctgaatgaccttttcccgaacgatttcaatagcgtgtttaaactcttgccttcgttttacagaaaactggaaaaggaaaatgggcgtaaaattgggtcgtgcttgatcggagccatgcctggctctttttattcacggctgtttccggaagaatctatgcatttcttacacagctgttattcagtgcattggctgtcacaggtcccgagtggattagtgacagaactgggcatctctgcaaacaaggggtgtatctattcatctaaagcaagccggcccccagtacagaagacgtaccttgaccaattcaccaaagactttacgactttcctgcgtattcactcggaagagctgatttcgcgcggaagaatgttgttgacttttatctgtaaggaagacgaattcggaaaccctaatagtatggacctgttagagatgtcaataaatgacttggtgatcgagggtcatctcgaagaggaaaagcttgattcctttaatgtcccagtttatgccgccagtgcagaagaagtgaagcgtatagtagaggaggaaggttccttcgagattctgtatttggagacgtttaaggccccgtacgacgctggcttctctatcgatgacgactatcaaggtcggagtcacagtccggtgtcatgtgatgagcatgcgagagcggctcatgttgcgagtgttgtacggagcgtgtacgagcctatcctggcaggacactttggtgaggcaatcttaccggacctctctcacagaatcgagaaaaatgctgcgaaagtattacgttctggcaaaggattctatgactcgctgatcataagtttggctaagaagccggaaaaatctgacgtgtaa

SEQ ID NO: 36 - protein, 385 aa, Artificial Sequence
  Methyl Transferase From Coffea arabica
MELQEVLQMNGGEGDTSYAKNSAYNQLVLAKVKPVLEQCVRELLRANLPNINKCIKVADLGCASGPNTLLTVRDIVQSIDKVGQEKKNELERPTIQIFLNDLFPNDFNSVFKLLPSFYRKLEKENGRKIGSCLIGAMPGSFYSRLFPEESMHFLHSCYSVHWLSQVPSGLVTELGISANKGCIYSSKASRPPVQKTYLDQFTKDFTTFLRIHSEELISRGRMLLTFICKEDEFGNPNSMDLLEMSINDLVIEGHLEEEKLDSFNVPVYAASAEEVKRIVEEEGSFEILYLETFKAPYDAGFSIDDDYQGRSHSPVSCDEHARAAHVASVVRSVYEPILAGHFGEAILPDLSHRIEKNAAKVLRSGKGFYDSLIISLAKKPEKSDV

SEQ ID NO: 37 - DNA, 1155 bp, Artificial Sequence
  Methyl Transferase From Coffea arabica
atggaactgcaggaagtgttgcacatgaacggcggcgagggcgatacctcctatgctaagaattcaagttataatctgtttctgattcgggttaaaccggtactggaacagtgcatacaagaattgctgcgtgcgaatttacctaacataaataagtgttttaaagtgggtgatcttgggtgtgcgtcaggaccgaacactttttctaccgttcgggacatcgtgcagtcaatcgataaagtcggtcaggagaaaaagaacgagttggaacgtccaacgatacaaatcttcctcaatgaccttttccagaacgactttaactcggtttttaagttactgccctccttctatcgtaatctggaaaaagagaatggccggaaaattggttcatgtctgattggtgcgatgccgggctcattttattcacgtttgtttccagaggaaagcatgcattttctccattcctgttactgcctccattggttgagccaggttccttctggccttgtcacagaactcggtatttctgtgaataaaggctgtatttattcctcgaaggcctccagaccacctatccagaaagcgtatttagatcagtttacaaaggattttaccacgtttctgagaattcactctgaagagctcatatcacgcggtagaatgctgttaacgttcatttgtaaagaagatgagttcgaccacccaaattcaatggatctgctggagatgtctataaatgatctggttgtcgagggtcatttagaagaggaaaaactggattcctttaacgtccccatttatgctcccagcacggaagaggtgaaacgcatcgttgaagaagaagggtcatttgaaatcttatatttagaaacgttttatgcaccttatgatgcgggcttttccatcgatgacgactatcaaggtcgctcccacagtccagtcagttgcgatgagcatgctcgggctgcacatgttgcttcagttgtgcgctctatttacgaaccaattcttgcatcacatttcggagaggctattcttccggacctttctcaccgcattgcgaaaaacgcagcaaaagtcttacgctcgggtaaggggttttatgattccgttataatcagtctcgcaaagaagcctgaaaaagctgatatgtaa

SEQ ID NO: 38 - protein, 384 aa, Artificial Sequence
  Methyl Transferase From Coffea arabica
MELQEVLHMNGGEGDTSYAKNSSYNLFLIRVKPVLEQCIQELLRANLPNINKCFKVGDLGCASGPNTFSTVRDIVQSIDKVGQEKKNELERPTIQIFLNDLFQNDFNSVFKLLPSFYRNLEKENGRKIGSCLIGAMPGSFYSRLFPEESMHFLHSCYCLHWLSQVPSGLVTELGISVNKGCIYSSKASRPPIQKAYLDQFTKDFTTFLRIHSEELISRGRMLLTFICKEDEFDHPNSMDLLEMSINDLVVEGHLEEEKLDSFNVPIYAPSTEEVKRIVEEEGSFEILYLETFYAPYDAGFSIDDDYQGRSHSPVSCDEHARAAHVASVVRSIYEPILASHFGEAILPDLSHRIAKNAAKVLRSGKGFYDSVIISLAKKPEKADM

SEQ ID NO: 39 - DNA, 1155 bp, Artificial Sequence
  Methyl Transferase From Coffea arabica
atggaactgcaggaggtcctgcacatgaatggtggtgagggtgatacctcgtacgccaaaaatagtttttataacctttttctgattcgtgtcaaacccatactcgagcagtgtatacaagaattactgcgcgctaacttgccgaatatcaacaagtgcataaaagttgccgacctgggttgcgcttcagggcctaatacgttgttgacagtgcgtgatattgttcagtctattgataaggtcggacaagagaagaaaaatgaactggaacgccctacaatccagatatttctgaatgatctgtttcagaacgactttaatagtgtgtttaaaagccttccgtcattctaccgcaagctggagaaagaaaatgggtgtaagattggcagttgtttgataggtgcgatgccaggaagcttttacggccggctgtttcccgaggaaagcatgcacttcttacacagttgttattgtttgcattggctgtcccaggtgcctagtgggttagtaaccgagctcgggatcagtgccaataaaggctgtatctactctagcaaagcgagccggcctccaatccaaaaagcgtacctggatcagtttactaaggactttacgacattcctgcgtatccatagcgaagaattaatctcgcgcggtcgcatgctgctgacctggatctgtaaggaagatgagttcgaaaacccaaatagtatagatctcctcgagatgagcattaacgatctcgtgatagagggtcacttagaggaggaaaaactggacagtttcaatgtcccaatctatgcgccatccacggaggaagtcaagtgcatagtggaagaagaaggctcctttgagattctttatcttgagactttcaaagtaccgtatgacgcgggatttagcatcgatgatgattaccaaggaagatctcactctccagtttcatgcgatgaacacgcaagagcggcgcatgtagccagcgttgtccgttctatatttgagccaattgtggcgtcccactttggcgaggctatcctgcctgatctgagccatcggatagccaagaatgcggctaaagttttacgttctggtaaagggttctatgattcagtcatcatctcgctggctaaaaagcctgagaaggcagatatgtaa

SEQ ID NO: 40 - protein, 384 aa, Artificial Sequence
  Methyl Transferase From Coffea arabica
MELQEVLHMNGGEGDTSYAKNSFYNLFLIRVKPILEQCIQELLRANLPNINKCIKVADLGCASGPNTLLTVRDIVQSIDKVGQEKKNELERPTIQIFLNDLFQNDFNSVFKSLPSFYRKLEKENGCKIGSCLIGAMPGSFYGRLFPEESMHFLHSCYCLHWLSQVPSGLVTELGISANKGCIYSSKASRPPIQKAYLDQFTKDFTTFLRIHSEELISRGRMLLTWICKEDEFENPNSIDLLEMSINDLVIEGHLEEEKLDSFNVPIYAPSTEEVKCIVEEEGSFEILYLETFKVPYDAGFSIDDDYQGRSHSPVSCDEHARAAHVASVVRSIFEPIVASHFGEAILPDLSHRIAKNAAKVLRSGKGFYDSVIISLAKKPEKADM

SEQ ID NO: 41 - DNA, 1161 bp, Artificial Sequence
  Methyl Transferase From Coffea liberica
atggaattgcaagaggttttacgcatgaatggcggtgaaggcgacactagctatgctaaaaattcgtcatacaacctgtttctgattcgggtcaaaccggtacttgaacaatgcattcaagggttactcagagccaacctgcctaacattaacaaatgcattaaagtagccgatttaggatgcgcaagtggaagcaatacattaagtacggtcagagatatagtgcaatctatagacaaggttggacaagaaaagaagaatgaattggaaagaccaacaattcaagtctttctgaacgacttgtttcagaacgactttaattccgtgtttaagctgttgccgtccttttatcggaaactggaaaaggaaaatggccggaaaattggaagttgcttgattggggccatgcctggttccttctatggtcggctgttcccagaggaaagtatgcactttcttcactcctgttattgcctgcattggctctctcaggtaccatcaggcctggttacagagttgggaatttcagcaaataaggggtgcatttattcgtccaaagccagcagaccgccgatcaaaaaggcctatcttgaccaattcacgaaggacttcactacgtttttacgggttcacagcgaagaattgataagtcgtggaagaatgttgttgacttggatctgtaaagaagacgagtttgaaaacccaaattcaattgatctgctggagatgtcgcttaatgatctggttattgagggccatctcgaggaagaaaaactggattcctttaacgtaccgatattcgccccgtccacggaagaggtcaaatgtatagtagaagaagaagggtcatttgagatactttacttagagacattcaaaacaccatacgacgcagggttttcaatcgacgatgattatcagggtagaagccattcgccggtttcatgtgacgaacatgcgattgcagaacatgtcgccgcagtggtgagatcgatttacgagcctatattagcttcacattttggtgaggcaattatgccggatcttagtcatcgtatagccaaaaatgctgcgaaggtcttgcgctcaggtaagggtttttatgactcaattatcataagtcttgccaagaagccggagaaatcagatgttaatcattaa

SEQ ID NO: 42 - protein, 386 aa, Artificial Sequence
  Methyl Transferase From Coffea liberica
MELQEVLRMNGGEGDTSYAKNSSYNLFLIRVKPVLEQCIQGLLRANLPNINKCIKVADLGCASGSNTLSTVRDIVQSIDKVGQEKKNELERPTIQVFLNDLFQNDFNSVFKLLPSFYRKLEKENGRKIGSCLIGAMPGSFYGRLFPEESMHFLHSCYCLHWLSQVPSGLVTELGISANKGCIYSSKASRPPIKKAYLDQFTKDFTTFLRVHSEELISRGRMLLTWICKEDEFENPNSIDLLEMSLNDLVIEGHLEEEKLDSFNVPIFAPSTEEVKCIVEEEGSFEILYLETFKTPYDAGFSIDDDYQGRSHSPVSCDEHAIAEHVAAVVRSIYEPILASHFGEAIMPDLSHRIAKNAAKVLRSGKGFYDSIIISLAKKPEKSDVNH

SEQ ID NO: 43 - DNA, 1155 bp, Artificial Sequence
  Methyl Transferase From Coffea canephora
atggaagtgcaggaagtccttagaatgaatggtggcgaaggtgatacatcatacgcgaaaaattcaagctataacctcttccttattcgggtcaaacccgtcctcgaacagtgcattcaagaactgttaagagcaaatctgcctaacataaataaatgtattaaagttgctgacctcggctgcgcgtctgggtcaaacactctttcaaccgtacggggcatagtccagatcatcgataaagtgggtcaggaaaagaaaaatgaacttgaacgcccgacaatacaggtgtttttaaacgatctgtttcaaaacgactttaactctgtctttaaatcacttccttctttctaccgcaagctggaaaaggaaaacggacgcaaaattggctcctgcttaatcggggcaatgccggggagcttttatggtcgtctgttccccgaagaaagcatgcacttcttgcattcatgttattgcttgcactggctgtctcaggtgccgagtggcttagtgactgagctgggtatttctgcgaacaaagggtgtatttattcgtcaaaggcgtcacggccacctataaagaaggcatatctggatcaattcacgaaggattttacaaccttcctgaaaatacacagtgaggagttgatttctcgcggtcgcatgctgctgacgtggatctgcaaggaagatgagtttgagaatcccaattcaattgatctgctcgaaatgtccctgaatgatttggtgacagagggcctcttggaagaagagaagctggactcttttaatgtgcctatctatgccccatcgacagaagtggttaaatgcattgtagaagaagaaggctcatttgaaatactctatttgaaaacattcaaagctccgtacgatgcaggattttcgaccgatgacgattatcaagggcgttctcatagcccagcgagttgtgacgaacatgctcgtgccgctcatgtagcttctgtggcacgctctatctttgagccgatagtagcctcccattttggagaagcaataatgccagacttgtcccatcgcatagctaaaaatgcggcaaaagttttacgcagcggaaagggctttttcgacagtattataattagcctggcgaaaaagccagaaaaatcagatatgtaa

SEQ ID NO: 44 - protein, 384 aa, Artificial Sequence
  Methyl Transferase From Coffea canephora
MEVQEVLRMNGGEGDTSYAKNSSYNLFLIRVKPVLEQCIQELLRANLPNINKCIKVADLGCASGSNTLSTVRGIVQIIDKVGQEKKNELERPTIQVFLNDLFQNDFNSVFKSLPSFYRKLEKENGRKIGSCLIGAMPGSFYGRLFPEESMHFLHSCYCLHWLSQVPSGLVTELGISANKGCIYSSKASRPPIKKAYLDQFTKDFTTFLKIHSEELISRGRMLLTWICKEDEFENPNSIDLLEMSLNDLVTEGLLEEEKLDSFNVPIYAPSTEVVKCIVEEEGSFEILYLKTFKAPYDAGFSTDDDYQGRSHSPASCDEHARAAHVASVARSIFEPIVASHFGEAIMPDLSHRIAKNAAKVLRSGKGFFDSIIISLAKKPEKSDM

SEQ ID NO: 45 - DNA, 1152 bp, Artificial Sequence
  Methyl Transferase From Coffea canephora
ctgcaggaagtcctgcacatgaacggcggagaaggagaagctagctatgctaagaattccagtttcaatcaactggtgctggccaaggtgaaacctgtcttagagcagtgcgtacgtgaactgctgcgggccaatttaccaaacatcaacaagtgtataaaggttgccgatttgggctgcgcttcgggcccaaatacactgttaacggtatgggatacagtacagtctattgataaggttaaacaggaaatgaaaaacgaactggagcgccccactattcaagtgtttctgacggaccttttccagaacgatttcaattccgtggtgatgttgttgccatccttttaccggaaactggagaaggagaacggacgtaaaatcggatcttgtctgatcgcggcaatgccgggctcttttcacggacgcttatttccagaagaatccatgcacttcttacattcctcttattccatccaattcctgtcacaagttccgagcgggttggttacggaactcggaatcacggcaaataaccgttcaatttattcgagtaaagcgtccccgcctccggtccagaaagcgtacttagaccagttcactaaagacttcacgacgtttttacggatgagatcggaggaactgctcagtcgtggcagaatgctgctgacctgcatttgcaagggggacgagtgcgatggtccaaatacaatggatttgttggaaatggctatcaacgacttagtagctgagggtcgtctgggtgaagaaaaattagattcatttaacgtgcccatctatacagcgtccgtggaagaagtcaaatgcatggtcgaggaagagggtagctttgagatactgtacctgcagacttttaaattacgctatgatgcaggttttagtatcgacgatgattgtcaagtacgctcccatagtccggtttattctgatgagcacgccagagctgcacatgtagcatctctcatacgctcagtttatgaaccaatcttagcatcccattttggtgaagtgataaaaccggatatcttccatcgttttgcgaccaacgcggcgaaagtaatacggctgggtaaaggcttttacaacaatttgataattagccttgccaagaaacccgagaaatcagatatttaa

SEQ ID NO: 46 - protein, 383 aa, Artificial Sequence
  Methyl Transferase From Coffea canephora
LQEVLHMNGGEGEASYAKNSSFNQLVLAKVKPVLEQCVRELLRANLPNINKCIKVADLGCASGPNTLLTVWDTVQSIDKVKQEMKNELERPTIQVFLTDLFQNDFNSVVMLLPSFYRKLEKENGRKIGSCLIAAMPGSFHGRLFPEESMHFLHSSYSIQFLSQVPSGLVTELGITANNRSIYSSKASPPPVQKAYLDQFTKDFTTFLRMRSEELLSRGRMLLTCICKGDECDGPNTMDLLEMAINDLVAEGRLGEEKLDSFNVPIYTASVEEVKCMVEEEGSFEILYLQTFKLRYDAGFSIDDDCQVRSHSPVYSDEHARAAHVASLIRSVYEPILASHFGEVIKPDIFHRFATNAAKVIRLGKGFYNNLIISLAKKPEKSDI

SEQ ID NO: 47 - DNA, 1158 bp, Artificial Sequence
  Methyl Transferase From Coffea arabica
atggaactccaggaggttctccacatgaatggtggcgaaggagaggcaagttacgcaaagaactcctcattcaatcaattggtcttagcgaaggtgaaacccgttctggaacaatgtgtgcgtgaactgcttcgcgcaaatcttccgaatatcaataaatgcattaaggtagcggatcttgggtgcgcgtcaggtccgaataccctcttaacggtctgggataccgttcaatccatcgataaagtcaagcaggaaatgaaaaatgagttggaacgcccgaccatacaggtgtttctgaccgatcttttccaaaacgattttaactcggtgttcatgttgctgccctcattttatcgcaaattagagaaggaaaatggacgcaaaattggctcttgtcttattgccgctatgcccggaagctttcatggtcggcttttcccagaggagtccatgcatttcttacattccagctactctttgcagtttctcagccaagtcccctctgggttggtcaccgagttagggataacagctaataaacgctcaatatactcttcgaaagcaagtccgcctccagtccagaaagcataccttgaccagttcaccaaagatttcacgacattcctccgcatgcgttccgaagagttactcagtcggggccgcatgttgcttacctgtatttgcaaaggtgatgaatgtgatgggcccaatacgatggacttgctggaaatggcgattaatgatttggtggctgaagggcgtctgggagaggagaaattggattcttttaacgttccaatctacacagccagcgttgaagaggttaaatgtatggtggaagaagaaggctctttcgaaatactctatcttcagacatttaaactgcggtatgatgccggtttttccattgacgatgactgccaagttagaagccattcgcccgtctacagcgatgaacatgcacgtgccgctcatgttgccagcttaattcgctcagtgtacgaaccgatcctggcgtcgcatttcggcgaggctataataccggatatatttcaccgttttgcaactaacgcggccaaggtgattcgcttggggaaagggttctacaacaaccttattatatcacttgctaagaaacctgaaaagagtgacatctaa

SEQ ID NO: 48 - protein, 385 aa, Artificial Sequence
  Methyl Transferase From
MELQEVLHMNGGEGEASYAKNSSFNQLVLAKVKPVLEQCVRELLRANLPNINKCIKVADLGCASGPNTLLTVWDTVQSIDKVKQEMKNELERPTIQVFLTDLFQNDFNSVFMLLPSFYRKLEKENGRKIGSCLIAAMPGSFHGRLFPEESMHFLHSSYSLQFLSQVPSGLVTELGITANKRSIYSSKASPPPVQKAYLDQFTKDFTTFLRMRSEELLSRGRMLLTCICKGDECDGPNTMDLLEMAINDLVAEGRLGEEKLDSFNVPIYTASVEEVKCMVEEEGSFEILYLQTFKLRYDAGFSIDDDCQVRSHSPVYSDEHARAAHVASLIRSVYEPILASHFGEAIIPDIFHRFATNAAKVIRLGKGFYNNLIISLAKKPEKSDI

SEQ ID NO: 49 - DNA, 1158 bp, Artificial Sequence
  Methyl Transferase From Coffea arabica
atggaactccaggaagttctgcacatgaacggtggcgaaggtgatgcttcgtatgctaaaaattcaagctttaatcagttggttctggctaaagtgaagccggtacttgaacaatgcgtgggagagctcttacgcgcaaatctccccaatattaacaaatgtataaaagttgcggatttagggtgtgcctccggcccgaacaccttgttaaccgtccgtgatatcgtacagtcaatcgataaagttagacaggaaatgaagaatgaattggaacgccccaccatccaagtctttcttacggacctgttccagaacgatttcaacagcgtgttcatgctgttaccgtcattctaccgcaaattagaaaaggagaacggtcggaagatcggttcttgtctgatagcagcaatgcccggctcttttcatggtcgtcttttccccgaagaatcgatgcatttcctccacagcagctattcactgcagttcttatcacaagttccgtcaggactggttactgagttgggtataactgcgaacaaacgctctatctatagctcaaaagcaagtccaccgccagtgcaaaaggcctacttggatcagtttactaaagactttacgaccttcctgcgcatacggagtgaagaactcctttctcgtggtagaatgttattgacatgcatctgcaaaggcgatgagttcgatggaccaaacactatggatctgttagaaatggcaataaacgatctcgtcgtggaaggccaccttgaggaagaaaaactggacagcttcaacgtccccatttatgctgcttcggttgaagagctgaagtgtattgtggaagaagagggttcatttgagatcttatatttagaaaccttcaaactccgctacgatgcgggtttttcgattgatgatgactgccaggtacgtagtcatagtccagaatattccgatgagcatgcaagagcagcgcatgtcgcgtcattactgcggtccgtatatgaaccgatcttagcgaaccactttggcgaagcgataatccctgatatctttcatcgttttgcgactaatgcagcgaaagtgattagattggggaagggtttttacaataacctcataattagcctggctaaaaagcccgagaagagcgatatctaa

SEQ ID NO: 50 - protein, 385 aa, Artificial Sequence
  Methyl Transferase From Coffea arabica
MELQEVLHMNGGEGDASYAKNSSFNQLVLAKVKPVLEQCVGELLRANLPNINKCIKVADLGCASGPNTLLTVRDIVQSIDKVRQEMKNELERPTIQVFLTDLFQNDFNSVFMLLPSFYRKLEKENGRKIGSCLIAAMPGSFHGRLFPEESMHFLHSSYSLQFLSQVPSGLVTELGITANKRSIYSSKASPPPVQKAYLDQFTKDFTTFLRIRSEELLSRGRMLLTCICKGDEFDGPNTMDLLEMAINDLVVEGHLEEEKLDSFNVPIYAASVEELKCIVEEEGSFEILYLETFKLRYDAGFSIDDDCQVRSHSPEYSDEHARAAHVASLLRSVYEPILANHFGEAIIPDIFHRFATNAAKVIRLGKGFYNNLIISLAKKPEKSDI

SEQ ID NO: 51 - DNA, 837 bp, Artificial Sequence
  Methyl Transferase From Coffea canephora
ctgcaagaagtccttcacatgaacggtggcgaaggggatactagctacgctaaaaactccagttacaatctggtgctgacaaaagttaaacctgtgctggaacaatgcattagagagctcctgagagcgaaccttccgaatattaataaatgcattaaagtagccgacttgggttgcgcttcaggccctaacacgcttctcactgttcgcgatattgtccagtccatagataaggttggtcaggaggagaagaatgaattggagcatcctaccattcagatcttcttgaatgacctttttcaaaatgatttcaacagcgtattcaagctgctccctagtttttatcggaaacttgagaaagagaacgggagaaaaataggcagttgtttaatctgggcaatgcctggcagcttttacagccggcttttcccagaagaaagtatgcactttcttcactcatgctattgtctgcagtggttaagtcaagtccctagtggcttagttacagagctgggtattagtgccaataaaggcataatatactcttctaaggcttctccaccaccggtgcagaaggcctatctggatcaattcaccaaagattttaccacatttctgcgtattcactctgaagaactcctttccagaggacgtatgctgttaacttgtatctgcaaaggagatgaaagtgacgggttgaacacaatagaccttctggagcgggcaattaacgacctcgtagtagaaggtttgttagaagaggaaaagttagattcattcaacttgccattatatactccatcgctcgaagtggtcaaatgcatggtggaagaataa

SEQ ID NO: 52 - protein, 278 aa, Artificial Sequence
  Methyl Transferase From Coffea canephora
LQEVLHMNGGEGDTSYAKNSSYNLVLTKVKPVLEQCIRELLRANLPNINKCIKVADLGCASGPNTLLTVRDIVQSIDKVGQEEKNELEHPTIQIFLNDLFQNDFNSVFKLLPSFYRKLEKENGRKIGSCLIWAMPGSFYSRLFPEESMHFLHSCYCLQWLSQVPSGLVTELGISANKGIIYSSKASPPPVQKAYLDQFTKDFTTFLRIHSEELLSRGRMLLTCICKGDESDGLNTIDLLERAINDLVVEGLLEEEKLDSFNLPLYTPSLEVVKCMVEE

SEQ ID NO: 53 - DNA, 837 bp, Artificial Sequence
  Methyl Transferase From Coffea canephora
ctgcaggaagtattgcacatgaatgaaggtgagggtgatacaagttatgccaagaatgcatcttataatctcgctttggcgaaagtgaagccatttcttgagcagtgcattcgtgaattgctccgtgcaaatcttccgaatatcaataagtgcattaaagtcgccgatcttggatgtgcttcaggaccaaatactctgcttacagtgcgggacatagtacaatccattgacaaagtaggacaggaggagaagaatgagctggaacgcccaacgatacagatattccttaatgatctgtttcagaacgatttcaatagcgtttttaagctgttaccctcattctacagaaaactggagaaggaaaacgggcgcaaaattgggtcttgcttaataagtgccatgcctggttccttctatggtcgcttatttcctgaagaaagtatgcactttctgcatagttgctattcggttcattggctttctcaggttccaagcggacttgttattgaattgggtatcggcgctaacaagggttccatttatagtagtaaaggatgtcgtccgccagttcagaaagcttatcttgatcaattcaccaaagactttacgacgttcttgcgtattcattccaaggagctgttttctcggggacgtatgctgctgacgtgtatatgcaaagtggatgaattcgatgaacccaatccgctggacctgctggatatggcaataaatgacctcatcgtagaaggcagattaggggaagagaaacttgattcatttaatgtccccatttataccgctagcgtcgaagaagtaaaatgcatggttgaagaataa

SEQ ID NO: 54 - protein, 278 aa, Artificial Sequence
  Methyl Transferase From Coffea canephora
LQEVLHMNEGEGDTSYAKNASYNLALAKVKPFLEQCIRELLRANLPNINKCIKVADLGCASGPNTLLTVRDIVQSIDKVGQEEKNELERPTIQIFLNDLFQNDFNSVFKLLPSFYRKLEKENGRKIGSCLISAMPGSFYGRLFPEESMHFLHSCYSVHWLSQVPSGLVIELGIGANKGSIYSSKGCRPPVQKAYLDQFTKDFTTFLRIHSKELFSRGRMLLTCICKVDEFDEPNPLDLLDMAINDLIVEGRLGEEKLDSFNVPIYTASVEEVKCMVEE

SEQ ID NO: 55 - DNA, 1098 bp, Artificial Sequence
  Methyl Transferase From Camellia sinensis
atggaagtaaaggaagcgctgttcatgaatagaggtgaaggcgaatcctcatacgctcaaaacagtagttttactcagaaggttgcgtctatgactatgccggtattggagaatgccgtagaaaccttatttagcaaggatttccatctttttcaggcactgaatgccgcggatctgggttgcgctacaagtccaaatacattcacggtgatttcgaccattaaacgtatgatggagaagaaatgccgcgaattgaactgtcagacgttagaactgcaagtttacttgaatgacctgcccggaaatgacttcaacactctgttcaaaggacttttgtctaaggtagtggtgggaaataagtgcgaagaagtctcatgctatgtgatgggcgtgccgggtagcttccatggtcggctttttcctagaaatagcttacaccttgtccactcatgctactcagcccactggttatctcaagctcctaaaggcctgacgtctcgggaaggtttaccgcttaacaaaggaaagatttacatttcaaaacgcagcccgccggtggtacgggaagcctatttgagtcagtttcatgatgattttacgatgtttcttaatgcacgttcccaggaagtggtgccgcatgggtgtatggtgctcatcctgcctagtagacaatccagcgatccaagttctatggaatcctgcttcacctgggaacttttggcaattgctattgccgaactcgtatcacaaggccttattgatgaggataaactggacaccttcaatgtaccatcatactttcccagtttagaagaagtaaaggatatcgtagagcgcgatggctctttcactatcgatcacatggagggtttcgagctggatacactgcagatgcaggaaaatgacaagtggatacgcggagagaaattagccaaagcagttagagcgttcacggagcctatcatttcaaaccagttcggccatgagataatggataagctgtatgacaaatttacacatatagtcgcgagcgatctggaagggaaaatccccaaatcaacgtccatagttttggtgctgagcaagattgtcggctaa

SEQ ID NO: 56 - protein, 365 aa, Artificial Sequence
  Methyl Transferase From Camellia sinensis
MEVKEALFMNRGEGESSYAQNSSFTQKVASMTMPVLENAVETLFSKDFHLFQALNAADLGCATSPNTFTVISTIKRMMEKKCRELNCQTLELQVYLNDLPGNDFNTLFKGLLSKVVVGNKCEEVSCYVMGVPGSFHGRLFPRNSLHLVHSCYSAHWLSQAPKGLTSREGLPLNKGKIYISKRSPPVVREAYLSQFHDDFTMFLNARSQEVVPHGCMVLILPSRQSSDPSSMESCFTWELLAIAIAELVSQGLIDEDKLDTFNVPSYFPSLEEVKDIVERDGSFTIDHMEGFELDTLQMQENDKWIRGEKLAKAVRAFTEPIISNQFGHEIMDKLYDKFTHIVASDLEGKIPKSTSIVLVLSKIVG

SEQ ID NO: 57 - DNA, 1098 bp, Artificial Sequence
  Methyl Transferase From Camellia sinensis
atggaggtgaaagaggcgctgttcatgaatcggggagaaggtgagaatagttatgcacaaaactcctcctttacacagaaggttgcgagtatgaccatgccggtactcgaaaacgctgtggaaacccttttctctaaagatttccatttgcttcaggcgctgaacgtagtagatctgggatgcgctacgagtccgaatactttcacggtaatcagcaccatcaagcggatgatggaaaagaagtgccgggaactcaattgtcagacacttgaactgcaggtttatctcaatgatctgccaggtaatgacttcaatacactgtttaaaggccttttgtctaaagttgtagtcggcaacaagtgcgaagaagtgtcatgctatgttatgggcgttcctgggtcatttcacggtcgtttatttccgagaaattcattgcgcttggttcactcttgctattctgctcactggttatctcaggcgcccaaggggttgacatcacgtgaaggtttagcattgaaccgccgcaaaatatacatttccaaaaccagccctctggtagtgcgtgaagcgtatctttcccagtttcacgaagatttcactatgttcttaaatgcccgtagtcaggaagtagttcccaatggctgcatggtcctgatacttccggggcgtcagtcttcaaatccgtcatcaatggagagctgctttacctgggaactgttagcgatagccataggagagctcgtgagccaaggcctcatcgatgaggacaagctcgatacatttaacgtgccgagttatttcccgagcttagaagaggttaaggacatagtcgagagagatggtagttttacaatagatcacatggttggcttcgaactggatacgccccagatgcaagagaatgataaatgggtacgtgtagaaaagttagctaaggcggttagagcttttactgagccaatcataagtaatcagtttggtcacgaaattatggacaaactctacgacaaattcacttacatcgtggtaagtgatttggaagggaagatcccgaaaactacgtcgatagtactggtgctgtcgaaaattatcggataa

SEQ ID NO: 58 - protein, 365 aa, Artificial Sequence
  Methyl Transferase From Camellia sinensis
MEVKEALFMNRGEGENSYAQNSSFTQKVASMTMPVLENAVETLFSKDFHLLQALNVVDLGCATSPNTFTVISTIKRMMEKKCRELNCQTLELQVYLNDLPGNDFNTLFKGLLSKVVVGNKCEEVSCYVMGVPGSFHGRLFPRNSLRLVHSCYSAHWLSQAPKGLTSREGLALNRRKIYISKTSPLVVREAYLSQFHEDFTMFLNARSQEVVPNGCMVLILPGRQSSNPSSMESCFTWELLAIAIGELVSQGLIDEDKLDTFNVPSYFPSLEEVKDIVERDGSFTIDHMVGFELDTPQMQENDKWVRVEKLAKAVRAFTEPIISNQFGHEIMDKLYDKFTYIVVSDLEGKIPKTTSIVLVLSKIIG

SEQ ID NO: 59 - DNA, 1098 bp, Artificial Sequence
  Methyl Transferase From Camellia sinensis
atgaaagaagtaaaagaagctctgtttatgaacaaaggcgaaggtgaatccagttacgctcagaatagctcgttcactcagaccgtaacgtccatgaccatgccggtgttagagaatgccgttgagacattattctccaaggattttcacttgctgcaagctcttaatgcagtagatttaggatgcgcagctggaccaactacatttacagtgatttcgactatcaaacgtatgatggagaagaaatgtcgcgaactcaattgtcaaactttagaacttcaggtatacctgaacgatttacctggtaacgactttaatacactgtttaaaggtcttccgtctaaagtcgtcggtaataaatgcgaagaagtcagctgctacgtagtaggtgttccaggttcatttcatggtcggttatttcctcgtaattcccttcacttggtgcattcatgttacagtgttcattggttgactcaggctccgaaaggtttgaccagtaaggaggggttagcgctcaacaaaggtaagatctatattagtaagacatcccctccagtggttagagaggcatacttgtctcagtttcacgaggattttactatgtttctgaactcacggagccaggaagttgttccaaatggctgcatggtattgattctgagaggtcgtctttcgagtgatcctagcgatatgggttcatgttttacttgggaactgctggccgtggcgatcgcagaactcgtgagccaaggtttaatcgatgaagacaagctggacacctttaacgtgccgtcatatttcccttcccttgaagaggtgaaagacatagttgaacgcaatggctctttcactattgaccacatggagggcttcgaattggattcacccgagatgcaggaaaatgataaatgggttcggggcgaaaaatttgctacagttgcgcgggcgttcacggaaccgattatttcaaatcagtttgggcacgaaatcatggataaactttatgagaaatttacacacatcgtggtgagtgatttcgaagccaaaataccgaaaattacttctataatcttggttctcagtaaaattgtgggctaa

SEQ ID NO: 60 - protein, 365 aa, Artificial Sequence
  Methyl Transferase From Camellia sinensis
MKEVKEALFMNKGEGESSYAQNSSFTQTVTSMTMPVLENAVETLFSKDFHLLQALNAVDLGCAAGPTTFTVISTIKRMMEKKCRELNCQTLELQVYLNDLPGNDFNTLFKGLPSKVVGNKCEEVSCYVVGVPGSFHGRLFPRNSLHLVHSCYSVHWLTQAPKGLTSKEGLALNKGKIYISKTSPPVVREAYLSQFHEDFTMFLNSRSQEVVPNGCMVLILRGRLSSDPSDMGSCFTWELLAVAIAELVSQGLIDEDKLDTFNVPSYFPSLEEVKDIVERNGSFTIDHMEGFELDSPEMQENDKWVRGEKFATVARAFTEPIISNQFGHEIMDKLYEKFTHIVVSDFEAKIPKITSIILVLSKIVG

SEQ ID NO: 61 - DNA, 1095 bp, Artificial Sequence
  Methyl Transferase From Camellia irrawadiensis
atggaggtaaaagaagccttgtttatgaacaagggagagggcgaaagttcctacgctcagagttctagttttacggaaacagttactagtatgactatgccagtgcttgagaatgctgttgaaactctgttctcaaaagatttccatcttttacaagcattgaacgcagccgaccttggctgtgcagcaggtcctaccacgtttacggtaatcagcaccataaaacgtatgatggagaagaagtgccgggagctgaattgtcagacgttagaactccaggtatacctgaacgacttgcccggcaatgacttcaacaccctgtttaaagggttgtcgagtaaggtggtgggcaacaagtgcgaagaagtgccatgttatgttgtaggcgtacccgggagctttcatggtagactttttccccgtaacagcctgcacttagttcactcgtgttattcagttcactggttgactcaggcacctaaaggactgactagtaaagagggtctggccctgaataaaggtaaaatctacataagcaagacctctccgccagtagtgcgggaagcctacttgtcacagtttcatgaggattttacaatgtttcttaattcgcgctcgcaagaagttgttccaaatgggtgtatggtattaattttacgtggacgtctgtcttccgatccgagcgacatggagtcatgttttacttgggaactcttagcagtggccatagctgagctggtgtcacagggtctgattgatgaggataagctcgacacatttaacgtaccgtcctattttccgagtctggaggaagttaaagatatagtcgagcgcaatggctccttcacgatagatcacatggagggtttcgaacttgacagtccacagatgcaggaaaatgacaaatgggttcgtggtgaaaagtttgcgacagtggcacgcgcttttacagagcccattattagcaatcagtttggccatgaaattatggacaaactctatgagaaattcacccacattgtagtgtcagacttagaagctaaaatcccgaaaataacctcaatcatcctggttttatcgaaaattgtaggctaa

SEQ ID NO: 62 - protein, 364 aa, Artificial Sequence
  Methyl Transferase From Camellia irrawadiensis
MEVKEALFMNKGEGESSYAQSSSFTETVTSMTMPVLENAVETLFSKDFHLLQALNAADLGCAAGPTTFTVISTIKRMMEKKCRELNCQTLELQVYLNDLPGNDFNTLFKGLSSKVVGNKCEEVPCYVVGVPGSFHGRLFPRNSLHLVHSCYSVHWLTQAPKGLTSKEGLALNKGKIYISKTSPPVVREAYLSQFHEDFTMFLNSRSQEVVPNGCMVLILRGRLSSDPSDMESCFTWELLAVAIAELVSQGLIDEDKLDTFNVPSYFPSLEEVKDIVERNGSFTIDHMEGFELDSPQMQENDKWVRGEKFATVARAFTEPIISNQFGHEIMDKLYEKFTHIVVSDLEAKIPKITSIILVLSKIVG

SEQ ID NO: 63 - DNA, 1098 bp, Artificial Sequence
  Methyl Transferase From Camellia japonica
atgatggaagtgaaagaagccctgtttatgaatcggggtgaaggggaaagcagctacgcgcaaaactcgtcatttactcaaaaagttgcatcattaaccatgccagttcttgagaatgccgtggagactctgttctcaaaagatttccaccttctgcaggcattgaatgccgccgatctgggttgtgccgcagggccgaacacatttaccgttatattcacaattaaacgcatgatggagaagaaatgtagagaactgaattgccagaccctggagctgcaagtgtatctcaacgacctgcctgggaacgattttaacacattgtttaagggtctgtcgtcaaaagtcgtaggtaacaaatgcgaagaggtatcgtactatgtcatgggagtgccaggttcctttcatggccgcttgtttccacgcaatagtctgcatctggtacactcgagttatagcgtacattggttgtctcaagcacctaagggtttgagatcccgcgaaggcttggctctgaataagggcaaaatttacatatcgaaaacgtctcctccggttgtccgggaagcctacttatcacaattccacgaggattttactatgttccttaatgcccgtagccaagaagtagtgcctaatggatgtatggtgcttatccttcacggtcgtaaatcatcagacccgtcgaatatggaaagttgctttacgtgggaactcttagccatcgcaattagcgagcttgtgtcacagggcttgatcgatgaggataaactcgataccttcaacgtgccatattatacgcctagccttgaggagatgaaagatatcgtagaacgtgaaggatcatttacaattgaccatatagagggctttgagcttgattccccgcacatgcaagaaaaagacaagtgggcgggtagagaaaagcttgcaaaagccatacgcgcttttaccgagccgattatctccaatcagttcggccatgagattatggacaagttatatgacaagttcacacatatagttgtctctgacttagaggctaaaatcccaaaaaccacctcaatcatcctggtcttgtctaaaattgttgggtaa

SEQ ID NO: 64 - protein, 365 aa, Artificial Sequence
  Methyl Transferase From Camellia japonica
MMEVKEALFMNRGEGESSYAQNSSFTQKVASLTMPVLENAVETLFSKDFHLLQALNAADLGCAAGPNTFTVIFTIKRMMEKKCRELNCQTLELQVYLNDLPGNDFNTLFKGLSSKVVGNKCEEVSYYVMGVPGSFHGRLFPRNSLHLVHSSYSVHWLSQAPKGLRSREGLALNKGKIYISKTSPPVVREAYLSQFHEDFTMFLNARSQEVVPNGCMVLILHGRKSSDPSNMESCFTWELLAIAISELVSQGLIDEDKLDTFNVPYYTPSLEEMKDIVEREGSFTIDHIEGFELDSPHMQEKDKWAGREKLAKAIRAFTEPIISNQFGHEIMDKLYDKFTHIVVSDLEAKIPKTTSIILVLSKIVG

SEQ ID NO: 65 - DNA, 1068 bp, Artificial Sequence
  Methyl Transferase From Camellia telotii
atgaatcggggcgaaggagaatctagcgatgcccagaatagctccttcacacagaaaggcgcatccatgactatgccggtgctcgagaatgcggtggaaaccctgttctcgaaagatttccatttgctgcaagcgttaaatgccgctgattttggatgtgctgccggcccgaatactgtcatttcgacaataaaaagaatgatggagaaaaagtgtcgcgagctgaattgtcagacgctcgagttgcaggtttacctgaacgatctcccgggtaatgacttcaacaccctgttcaagggtcttagctcaaaaattgttgtgggaaacaagtgcgaggaggtaagttgttatgcgatgggcgttccgggctctttccacggaagattatttccccgtaactcgctccatctggtgcattcaagttattccgtccattggctctcacaggcaccgaaaggcttaagatcacgcgagggactggccctcaacaaaggcaaaatttacatatcaaaaacatcacctccggttgttcgcgaagcgtatttaagccagttccatgaagactttacgatgttcttaaacgccagaagtcaagaggtggtaccgaacgggtgcatggtgttaattttgcatggcagaaagtcttccgacccgtctaacatggaatcgtgtttcacttgggaactgttagctatcgcgatcgcggagctggtatcacaagggctcattgacgaggacaagcttgacacgtttaatgtgccatattacacgcctagtctggaagaaatgaaagatattgtagaacgcgaggggtcttttaccattgaccacattgagggtgtggaattagatagcccccacatgcaagaaaaagataaatgggcaggcagagagaagttagccaaagctattcgcgcgtttactgaaccaatcatctcaaaccagttcggtcatgagatcatggataaactgtacgataagtttacgcacattgttgtaagtgatctggaggcaaaaataccaaaaactacttcgattatcctcgtcctgtcaaaaatagttggctaa

SEQ ID NO: 66 - protein, 355 aa, Artificial Sequence
  Methyl Transferase From Camellia telotii
MNRGEGESSDAQNSSFTQKGASMTMPVLENAVETLFSKDFHLLQALNAADFGCAAGPNTVISTIKRMMEKKCRELNCQTLELQVYLNDLPGNDFNTLFKGLSSKIVVGNKCEEVSCYAMGVPGSFHGRLFPRNSLHLVHSSYSVHWLSQAPKGLRSREGLALNKGKIYISKTSPPVVREAYLSQFHEDFTMFLNARSQEVVPNGCMVLILHGRKSSDPSNMESCFTWELLAIAIAELVSQGLIDEDKLDTFNVPYYTPSLEEMKDIVEREGSFTIDHIEGVELDSPHMQEKDKWAGREKLAKAIRAFTEPIISNQFGHEIMDKLYDKFTHIVVSDLEAKIPKTTSIILVLSKIVG

SEQ ID NO: 67 - DNA, 1101 bp, Artificial Sequence
  Methyl Transferase From Camellia granthamiana
atgatggaagttaaggaagcactgttcatgaatggtggtgagggcgaatcctcttatgcgcaaaattccagtttcacacagaaagtggcgagtatgacaattcctgtgttggaaatagctgtggagacaatattttctaaggatttccatcttttacaagcacttaatgccgctgatctggggtgcgcagccggaccaaatacatttacggttatttcaactattaagcgcatgatggaaaagaaatgccgggagctcaactgcgaaactttggaattacaagtatatcttaatgatcttcccggcaatgacttcaatacgttgtttaaaggcctgagcagtacggatgtggttggaaataagtgcgaagaagtttcctgttatgttatgggtgtcccaggctcgtttcatggtcgtctgtttccgcgtaactcattgcatttggtacatagttcttatagcgttcattggttatcccaggccccaaagggcttacggtctagagaaggcctggcattgaacaaaggtaaaatctacattagtaagacctcaccacctgttgttagagaggcgtatctgagtcagttccatgaagatttcacaatgttcttaaacgcccgtagtcaagaagtggttcctaatgggtgtatggtcctcattctccatggaagaaaatcatccgatccctccaaaattgaatgctgctttacttgggaattattggccattgctatcgccgaacttgtttcccaggggctgatcgacgaggataaattagacaccttcaacgtgccatactacacgccatctctggaagaggttaaggacattgtagaacgcgagggctcgttcactatcgatcacatggagggcctggaattagactctccacaaatgcaggaaaaagataaatgggtacggggtgaaaaactggcgaaagcggtacgcgcattcacggaaccaataatatccaaccagtttggtcacgaaattatggacaagctttatgacaaattcactcacatcgtagtatcagaccttgaagccaaaaagcctaaaactaccagtataattttggtgctgagcaaaattgttggctaa

SEQ ID NO: 68 - protein, 366 aa, Artificial Sequence
  Methyl Transferase From Camellia granthamiana
MMEVKEALFMNGGEGESSYAQNSSFTQKVASMTIPVLEIAVETIFSKDFHLLQALNAADLGCAAGPNTFTVISTIKRMMEKKCRELNCETLELQVYLNDLPGNDFNTLFKGLSSTDVVGNKCEEVSCYVMGVPGSFHGRLFPRNSLHLVHSSYSVHWLSQAPKGLRSREGLALNKGKIYISKTSPPVVREAYLSQFHEDFTMFLNARSQEVVPNGCMVLILHGRKSSDPSKIECCFTWELLAIAIAELVSQGLIDEDKLDTFNVPYYTPSLEEVKDIVEREGSFTIDHMEGLELDSPQMQEKDKWVRGEKLAKAVRAFTEPIISNQFGHEIMDKLYDKFTHIVVSDLEAKKPKTTSIILVLSKIVG

SEQ ID NO: 69 - DNA, 1101 bp, Artificial Sequence
  Methyl Transferase From Camellia lutchuensis
atgatggaggtgaaagaggccctttttatgaatggtggcgagggagaatcgtcttacgcccagaacagttcatttacgcaaaaggttgcctctatgactatgcctgtgcttgaaattgcagttgaaacgttactgagcaaagacttccatttactgcaggccctgaacgtagcggatctggggtgtgcggccggtccgaatacatttaccgtaatatccaccattaaacggatgatggaaaagaaatgtcgcgaactgaattgtcagacgctggaattgcaagtttaccttaacgatctgccggggaatgattttaacacactgttcaaaggcctcagctcaaaggttgttgtaggtaacaaatgcgaggaagttagctgttatgctatgggcgtcccaggtagcttccacggtagattatttccacggaattcgttacacttggtccactccagctattcagtgcactggctgtcccaagcacctaaaggcctccggtcaagagaagggctggcgcttaacaaaggtaaaatctacataagtaagacatctcccccagttgttagagaagcgtatttgtcccagtttcatgaagactttacaatgttcttgaatgctcgctcccaggaggttgtacccaatggatgcatggtcctgatcttgcacggaagaaaatcaagtgatcctagcaacatggagtcatgcttcacatgggaactgttagcgattgcgattgccgggcttgtctcacagggcttaatagacgaagacaaactcgatacgtttaacgtgccctattacaccccgagcctcgaagaagtaaaagacatagtggaacgcgaaggcagtttcactatcgatcacatggaaggattagaattagataatcctcacatgcaggaaaaagacaaatgggtacgtggtgctaaactggctaaagctgtgcgcgcgtttacggagccgattatcagcaaacaattcggccatgagattatgaataagctgtatgataaattcacacatattgtcgttagtgagctcgaggccaagaaacctaagactaccagtataattcttgtcttgagcaaaatagttgggtaa

SEQ ID NO: 70 - protein, 366 aa, Artificial Sequence
  Methyl Transferase From Camellia lutchuensis
MMEVKEALFMNGGEGESSYAQNSSFTQKVASMTMPVLEIAVETLLSKDFHLLQALNVADLGCAAGPNTFTVISTIKRMMEKKCRELNCQTLELQVYLNDLPGNDFNTLFKGLSSKVVVGNKCEEVSCYAMGVPGSFHGRLFPRNSLHLVHSSYSVHWLSQAPKGLRSREGLALNKGKIYISKTSPPVVREAYLSQFHEDFTMFLNARSQEVVPNGCMVLILHGRKSSDPSNMESCFTWELLAIAIAGLVSQGLIDEDKLDTFNVPYYTPSLEEVKDIVEREGSFTIDHMEGLELDNPHMQEKDKWVRGAKLAKAVRAFTEPIISKQFGHEIMNKLYDKFTHIVVSELEAKKPKTTSIILVLSKIVG

SEQ ID NO: 71 - DNA, 1098 bp, Artificial Sequence
  Methyl Transferase From Camellia irrawadienensis
atgggcaaagttaacgaagttttattcatgaaccgcggagagggggagatctcatacgcacagaattcagcgtttacccaaaaagttgcgagcatggcaatgccggccttggagaacgcggttgagactttgtttagcaaagactttcatttacttcaggcacttactgctgctgatcttggctgtgctgccgggccgaataccttcgccgttatttctacgattaagcggatgatggagaagaaatgtagagaattatactgccaaacgctcgaactgcaggtatatttgaacgacctgttcggaaatgatttcaataccctcttcaaaggcctctcatcagaagttgtcggcaataagtgcgaagaagtatcctgctatgttatgggtgttcctgggtcattccatggacgtttatttccccgcaattcattgcatttagttcactcaagctactcagtgcattggctgacccaggccccaaaaggcttaacaagtcgggaaggcctcgcattgaacaaaggcaagatatatatctctaaaacatcaccgccagttgtgaaagaagcgtacctgtcgcagtttcacgaagattttactatgttcttaaatgcgcgctctcaggaagtagtgcccaatggttgcatggttttgatcctgcatggacgtcaaagttccgatccctcagaaatggagtcctgctttacttgggagctgctcgcgattgctattgcagagttagtgtcgcaaggtttgatcgatgaagacaaattggatacctttaacgtgccaagctattggccatctcttgaagaagtgaaagatatcgtcgaacgtgatggcagtttcactattgaccatttggagggtttcgaattggactctctggagatgcaggaaaacgacaaatgggttcgcggagataaatttgctaagatggtgcgggcctttactgaaccgattatttcaaaccagttcggccacgagattatggacaaactgtatgataaatttacccacattctggttagtgaccttgaagcagaactgccgaagacaaccagcattattctggtattgtcgaagatagttgggtaa

SEQ ID NO: 72 - protein, 365 aa, Artificial Sequence
  Methyl Transferase From Camellia irrawadienensis
MGKVNEVLFMNRGEGEISYAQNSAFTQKVASMAMPALENAVETLFSKDFHLLQALTAADLGCAAGPNTFAVISTIKRMMEKKCRELYCQTLELQVYLNDLFGNDFNTLFKGLSSEVVGNKCEEVSCYVMGVPGSFHGRLFPRNSLHLVHSSYSVHWLTQAPKGLTSREGLALNKGKIYISKTSPPVVKEAYLSQFHEDFTMFLNARSQEVVPNGCMVLILHGRQSSDPSEMESCFTWELLAIAIAELVSQGLIDEDKLDTFNVPSYWPSLEEVKDIVERDGSFTIDHLEGFELDSLEMQENDKWVRGDKFAKMVRAFTEPIISNQFGHEIMDKLYDKFTHILVSDLEAELPKTTSIILVLSKIVG

SEQ ID NO: 73 - DNA, 1083 bp, Artificial Sequence
  Methyl Transferase From Camellia sinensis
atggaactcgctactgccggtaaggttaatgaggttctgtttatgaacagaggcgagggcgaatctagttacgcacaaaacagctcatttacacagcaagtcgcgagcatggcccagcccgcgctcgaaaatgcagttgaaacgctgtttagcagagattttcacttacaagcactcaacgctgcagatctgggatgtgctgcaggcccgaacacgtttgcggtaatctcaaccatcaaacgcatgatggagaaaaagtgccgggaattgaactgtcagaccctggagttacaggtgtacctgaatgacctgttcggcaatgactttaatactctgttcaagggcctcagcagtgaggtaataggtaataagtgcgaagaagtgagctgctacgtaatgggagtgccaggtagttttcatggacggcttttcccgcgcaattctcttcacctcgttcactcgagctatagcgttcattggttaacacaagcaccaaagggattaacaagcagagagggtttagccttgaacaaaggaaaaatctacatcagcaaaacttctccgcccgtagttagagaagcgtacctgtcacaatttcacgaggactttaccatgttcctcaatgcgagatctcaggaggtggtccccaacggctgcatggttctcattctgcgcgggagacaatgctcagatccatcagacatgcaatcttgtttcacctgggagctgttagccatggccatagccgagctcgtttcgcaaggcttgatagatgaagacaagttggacacttttaatataccctcatattttgctagcctggaggaggtaaaggacattgtggagcgggacggcagttttacaatcgaccatatagaggggttcgatctcgattcacttgagatgcaggagaacgacaaatgggtccgcggcgaaaaatttactaaagtagtgcgggcctttacagaacccattatctcaaatcagtttggacacgaaatcatggataagctgtatgataagttcactcacatcgtggtcagcgacttggaggcgaaacttcccaaaactaccagtatctaa

SEQ ID NO: 74 - protein, 360 aa, Artificial Sequence
  Methyl Transferase From Camellia sinensis
MELATAGKVNEVLFMNRGEGESSYAQNSSFTQQVASMAQPALENAVETLFSRDFHLQALNAADLGCAAGPNTFAVISTIKRMMEKKCRELNCQTLELQVYLNDLFGNDFNTLFKGLSSEVIGNKCEEVSCYVMGVPGSFHGRLFPRNSLHLVHSSYSVHWLTQAPKGLTSREGLALNKGKIYISKTSPPVVREAYLSQFHEDFTMFLNARSQEVVPNGCMVLILRGRQCSDPSDMQSCFTWELLAMAIAELVSQGLIDEDKLDTFNIPSYFASLEEVKDIVERDGSFTIDHIEGFDLDSLEMQENDKWVRGEKFTKVVRAFTEPIISNQFGHEIMDKLYDKFTHIVVSDLEAKLPKTTSI

SEQ ID NO: 75 - DNA, 1059 bp, Artificial Sequence
  Methyl Transferase From Paullinia cupana var sorbilis
atgaataccggtgagggtgaaagctcatatcttcttaactccaaatttaccaacgtgacggcaatcaagagcatccctaccctgaagcgcgccattgaatcactctttaaagaggaaagcccgccctttgagcatttactgaacgtagcggatcttggatgtgcgtctggatcaacatcgaataccattatgccaaccgtagtccagacggtggttaataaatgccgtgaactcaaccataaaattccagaattccaattctatctgaatgatctccctagcaacgactttaatactctgttcaagggtctgaacggatttgtgggaagcggaggagaagagttcgagaatacgtcttgccttgtaatgggtgcacctggcagttttcatgggcgcttatttccactcaacacgatacatctggtatactcaaattattcggttcactggttgagtaaagttcccgatttgcgcgacgaaaaaggtaaccccatcaacaaagggacattctacattagcaaaacaagccccagtggagtgcgtgaagcttatttggcacaatttcagaaagattttacattattcttgaagtcccgtgcagaagaaatggtttcaaacggtcgcgtagttctcgtcttgcacggtcgtctgagtcaagactttagctgcgaaaaagagcttcaactgccctggctgattttgagtcaagccatatcgcgcctggtatcaaaaggcttgatcgacgaagagaaactcgactcatttgaagtaccgtactatactccaagcgtgcaggaagtgaaggaattggtcgaaggagaagggtcgtacgctgtagagttgatggaaacttttactatccgtatcggggctcggaatgaaggtatttggtccgatgcaagaggttttggaaataacctgcgttccatcacggaaacgatgataagccaccacttcggcccgcagatactcgatgagttgtacgatgagattcaggatttacctctgcaggactttgcaacacagtgctcattcgttgtcggcttaaaaagaaattaa

SEQ ID NO: 76 - protein, 352 aa, Artificial Sequence
  Methyl Transferase From Paullinia cupana var sorbilis
MNTGEGESSYLLNSKFTNVTAIKSIPTLKRAIESLFKEESPPFEHLLNVADLGCASGSTSNTIMPTVVQTVVNKCRELNHKIPEFQFYLNDLPSNDFNTLFKGLNGFVGSGGEEFENTSCLVMGAPGSFHGRLFPLNTIHLVYSNYSVHWLSKVPDLRDEKGNPINKGTFYISKTSPSGVREAYLAQFQKDFTLFLKSRAEEMVSNGRVVLVLHGRLSQDFSCEKELQLPWLILSQAISRLVSKGLIDEEKLDSFEVPYYTPSVQEVKELVEGEGSYAVELMETFTIRIGARNEGIWSDARGFGNNLRSITETMISHHFGPQILDELYDEIQDLPLQDFATQCSFVVGLKRN

SEQ ID NO: 77 - DNA, 405 bp, Artificial Sequence
  Methyl Transferase From Paullinia cupana var sorbilis
atggatatgaaagacgtgctttgcatgaataccggtgagggcgagagttcgtacttgctgaatagcaagttcacaaacgtaacagctatcaaatctatccccactcttaagagagcaattgaatcgctcttcaaggaagaatcccctccatttgaacacttattgaatgtagccgatttaggatgcgcctccggcagtacgagtaatacgataatgcctaccgtggtccaaacggtggtaaacaaatgtcgcgaactgaaccacaaaattcctgaatttcaattctacctgaatgacttgccgtcaaatgacttcaatacgttgttcaagggacttaatggatttgtaggtagcggtggtgaggaatttgaaaatacgagttgtctcgtgatgggtgcgccctaa

SEQ ID NO: 78 - protein, 134 aa, Artificial Sequence
  Methyl Transferase From Paullinia cupana var sorbilis
MDMKDVLCMNTGEGESSYLLNSKFTNVTAIKSIPTLKRAIESLFKEESPPFEHLLNVADLGCASGSTSNTIMPTVVQTVVNKCRELNHKIPEFQFYLNDLPSNDFNTLFKGLNGFVGSGGEEFENTSCLVMGAP

SEQ ID NO: 79 - DNA, 654 bp, Artificial Sequence
  Methyl Transferase From Camellia sinensis
atggaactggcaaccgcaggaaaagtaaatgaagtactctttatgaaccgcggcgaaggagaaagctcgtatgcccagaattcaagctttacacagcaagttgcttctatggcccagccagctcttgaaaacgctgtcgaaactttgttctcccgggattttcatctccaggctcttaacgctgctgatttggggtgcgctgcaggacctaatactttcgcggtcatctcaacaattaaacgcatgatggagaagaagtgtcgcgaactcaactgccagacgctcgaactccaagtctacctcaacgacctctttgggaacgactttaacacgctgtttaaaggcttgtcgagtgaagtcatcggaaataagtgtgaggaggttagttgctacgtgatgggagtaccggggtcattccacggtcgtctgtttcccagaaactctctgcatcttgtccattcctcatactcagtacattggcttactcaggccccaaagggccttacaagccgtgaaggtctggcactgaacaagggcaaaatttatatatccaagactagccctcccgttgtaagagaatcctatttatcccagttccatgaggattcccaatgttttagcatgttagacccacgcgggggctcacagtggtaa

SEQ ID NO: 80 - protein, 217 aa, Artificial Sequence
  Methyl Transferase From Camellia sinensis
MELATAGKVNEVLFMNRGEGESSYAQNSSFTQQVASMAQPALENAVETLFSRDFHLQALNAADLGCAAGPNTFAVISTIKRMMEKKCRELNCQTLELQVYLNDLFGNDFNTLFKGLSSEVIGNKCEEVSCYVMGVPGSFHGRLFPRNSLHLVHSSYSVHWLTQAPKGLTSREGLALNKGKIYISKTSPPVVRESYLSQFHEDSQCFSMLDPRGGSQW

SEQ ID NO: 81 - DNA, 1092 bp, Artificial Sequence
  Methyl Transferase From Theobroma cacao
atggaagtgaaagagatgttattcatgaataaaggcgatggcgaaaattcatacgtgaaaacatcgggttacactcagaaagtggcggccgtgacgcaacccgttgtatatcgggcagctcagagcctgttcactggtcggaattcttgtagttatcaggttctcaatgtcgcggatttagggtgctcaagcggacctaatacctttacagtgatgagcacagtgattgaatcaactcgtgacaaatgcagtgagctcaactggcagatgccagaaatccagttttatctgaatgatctggtcggtaacgattttaacaccttgtttaaaggcctgtcagttatacaggataagtacaaaaatgtaagctgtttcgccatgggagctccgggctcatttcatggacgtttatttccccaaaacagcatgcacctcatacatagcagctatggggtacagtggctctctaaagtaccgaaaatgacctcagaaggtggtttatcgccgccaaataaaggcaaaatttatatctcgaaaacgtctccgcccgccgtgtggaaagcttacctcagtcaatttcaggaggactttttaagtttcttgcgttgtcgcagtccggagctggttccagatggacgcatggtgctcattattcatggccgtaaatcagctgacccgaccactagagaaagctgttatacatgggaagtgcttgcagatgctatatcctaccaggtatcgcaaggcttaattgacgaggaaaaactgaactccttcaatgtcccttactatattcctagtcaggaagaggtacgggatttggttaataaagaaggcagcttcttaacggagtttgtagacacaatcgaagtagaactggagggaatctggactggcccggaaaacggtgccaaaaacctgcgcagcttcacagaaccaatgatttcccatcagttcggtgaggaagtcatggacaagctgtatgacaaggtcaaagatattctggttgaggactgcaaacaagaaaaacaatctacgagaggagtctctatagtacttgagctcaaaaagaaagagagccaccttagctaa

SEQ ID NO: 82 - protein, 363 aa, Artificial Sequence
  Methyl Transferase From Theobroma cacao
MEVKEMLFMNKGDGENSYVKTSGYTQKVAAVTQPVVYRAAQSLFTGRNSCSYQVLNVADLGCSSGPNTFTVMSTVIESTRDKCSELNWQMPEIQFYLNDLVGNDFNTLFKGLSVIQDKYKNVSCFAMGAPGSFHGRLFPQNSMHLIHSSYGVQWLSKVPKMTSEGGLSPPNKGKIYISKTSPPAVWKAYLSQFQEDFLSFLRCRSPELVPDGRMVLIIHGRKSADPTTRESCYTWEVLADAISYQVSQGLIDEEKLNSFNVPYYIPSQEEVRDLVNKEGSFLTEFVDTIEVELEGIWTGPENGAKNLRSFTEPMISHQFGEEVMDKLYDKVKDILVEDCKQEKQSTRGVSIVLELKKKESHLS

SEQ ID NO: 83 - DNA, 1119 bp, Artificial Sequence
  Methyl Transferase From Coffea canephora
atggagctccaagaagtcctgcggatgaatggaggcgaaggcgatacaagctacgccaagaattcagcctacaatcaactggttctcgccaaggtgaaacctgtccttgaacaatgcgtacgggaattgttgcgggccaacttgcccaacatcaacaagtgcattaaagttgcggatttgggatgcgcttctggaccaaacacacttttaacggttcgggacattgtccaaagtattgacaaagttggccaggaaaagaagaatgaattagaacgtcccaccattcagatttttctgaatgatcttttcccaaatgatttcaattcggttttcaagttgctgccaagcttctaccgcaaacttgagaaagaaaatggacgcaaaataggatcgtgcctaataggggcaatgcccggctctttctacagcagactcttccccgaggagtccatgcattttttacactcttgttactgtcttcaatggttatctcaggttcctagcggtttggtgactgaattggggatcggcacgaacaaagggagcatttactcttccaaagcaagtcgtctgcccgtccagaaggcatatttggatcaatttacgaaagattttaccacatttctaaggattcattcggaagagttgttttcacatggccgaatgctccttacttgcatttgtaaaggagttgaattagacgcccggaatgccatagacttacttgagatggcaataaacgacttggttgttgagggacatctggaggaagaaaaattggatagtttcaatcttccagtctatataccttcagcagaagaagtaaagtgcatagttgaggaggaaggttcttttgaaattttatacctggagacttttaaggtcctttacgatgctggcttctctattgacgatgaacatattaaagcagagtatgttgcatcttccgttagagcagtttacgaacccatcctcgcaagtcattttggagaagctattatacctgacatattccacaggtttgcgaagcatgcagcaaaggttctccccttgggcaaaggcttctataataatcttatcatttctctcgccaaaaagccagagaagtcagacatgtaa

SEQ ID NO: 84 - protein, 372 aa, Artificial Sequence
  Methyl Transferase From Coffea canephora
MELQEVLRMNGGEGDTSYAKNSAYNQLVLAKVKPVLEQCVRELLRANLPNINKCIKVADLGCASGPNTLLTVRDIVQSIDKVGQEKKNELERPTIQIFLNDLFPNDFNSVFKLLPSFYRKLEKENGRKIGSCLIGAMPGSFYSRLFPEESMHFLHSCYCLQWLSQVPSGLVTELGIGTNKGSIYSSKASRLPVQKAYLDQFTKDFTTFLRIHSEELFSHGRMLLTCICKGVELDARNAIDLLEMAINDLVVEGHLEEEKLDSFNLPVYIPSAEEVKCIVEEEGSFEILYLETFKVLYDAGFSIDDEHIKAEYVASSVRAVYEPILASHFGEAIIPDIFHRFAKHAAKVLPLGKGFYNNLIISLAKKPEKSDM

(19) United States
(12) Patent Application Publication
(10) Pub. No.: US 2017/0362616 A1
(43) Pub. Date: Dec. 21, 2017

(54) BIOSYNTHETIC PRODUCTION OF CAFFEINE

(71) Applicants: Codexis, Inc., Redwood City, CA (US); THE COCA-COLA COMPANY, Atlanta, GA (US)

(72) Inventors: Jonathan Vroom, South San Francisco, CA (US); Oscar Alvizo, Fremont, CA (US); Pankaj Soni, Kennesaw, GA (US); Michael D. Brown, Lilburn, GA (US); David Entwistle, San Carlos, CA (US)

(21) Appl. No.: 15/525,520

(22) PCT Filed: Nov. 24, 2015
(86) PCT No.: PCT/US2015/062324

§ 371 Date: May 9, 2017

Related U.S. Application Data
(60) Provisional application No. 62/084,797, filed on Nov. 26, 2014.

(51) Int. Cl. C12P 17/18; C12N 9/78; C12N 9/10

(52) U.S. Cl. CPC C12P 17/182; C12Y 305/04003; C12N 9/1007; C12N 9/78

(57) ABSTRACT

The present invention provides an enzymatic means for the biosynthetic production of caffeine. The present invention provides biosynthetic methods for production of caffeine comprising: providing guanine, a guanine deaminase, at least one methyl transferase, and a methyl donor; contacting the guanine with the gtheuanine deaminase to produce xanthine; contacting the xanthine with the methyl transferase and a methyl donor, under conditions wherein the xanthine is methylated, to produce a monomethylxanthine; contacting the monomethylxanthine with the methyl transferase and a methyl donor, under conditions wherein the monomethylxanthine is methylated, to produce a dimethylxanthine; and contacting the dimethylxanthine with the methyl transferase and a methyl donor, under conditions wherein the dimethylxanthine is methylated, to produce caffeine (i.e., 1,3,7-trimethylxanthine).

The present application claims priority to U.S. Prov. Pat. Appln. Ser. No. 62/084,797, filed Nov. 26, 2014, hereby incorporated by reference in its entirety, for all purposes.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “CX8-146WO1_ST25.txt”, a creation date of Nov. 23, 2015, and a size of 196 kilobytes. The Sequence Listing filed via EFS-Web is part of the specification and is incorporated in its entirety by reference herein.

### FIELD OF THE INVENTION

The present invention provides an enzymatic means for the biosynthetic production of caffeine.

### BACKGROUND OF THE INVENTION

Caffeine is a purine alkaloid that is contained within Theaceae plants (e.g., Camellia sinensis) and Rubiaceae plants (e.g., Coffea arabica). It is commonly used in beverages and food, as well as a raw material for some medicaments. Typically, caffeine is produced by extraction from caffeine-producing plants or by organic synthesis. The chemical pathway used to synthesize caffeine involves the methylation of xanthosine to produce 7-methylxanthine, which is then methylated to produce theobromine, which is then methylated to produce caffeine as an end-product. While use of this pathway successfully results in the production of commercially useful caffeine, there remains a need in the art for a synthetic pathway that is economical and environmentally friendly.

### SUMMARY OF THE INVENTION

The present invention provides enzymatic means for the biosynthetic production of caffeine.

The present invention provides biosynthetic methods for production of caffeine comprising: providing guanine, a guanine deaminase, at least one methyl transferase, and a methyl donor; contacting the guanine with the gtheuanine deaminase to produce xanthine; contacting the xanthine with the methyl transferase and a methyl donor, under conditions wherein the xanthine is methylated, to produce a monomethylxanthine; contacting the monomethylxanthine with the methyl transferase and a methyl donor, under conditions wherein the monomethylxanthine is methylated, to produce a dimethylxanthine; and contacting the dimethylxanthine with the methyl transferase and a methyl donor, under conditions wherein the dimethylxanthine is methylated, to produce caffeine (i.e., 1,3,7-trimethylxanthine).

The present invention provides biosynthetic methods for production of caffeine comprising: providing guanine, a guanine deaminase, at least one methyl transferase, and a methyl donor; contacting the guanine with the guanine deaminase to produce xanthine; contacting the xanthine with the methyl transferase and a methyl donor, under conditions wherein the xanthine is methylated, to produce 7-methylxanthine; contacting the 7-methylxanthine with the methyl transferase and a methyl donor, under conditions wherein the 7-methylxanthine is methylated, to produce theobromine; and contacting the theobromine with the methyl transferase and a methyl donor, under conditions wherein the theobromine is methylated, to produce caffeine. In some embodiments, the methyl transferase is selected from XMT, MXMT, and DXMT. In some further embodiments, the methods comprise at least two methyl transferases selected from XMT, MXMT, and/or DXMT. In some alternative embodiments, the methods comprise the methyl transferases XMT, MXMT, and DXMT.

The present invention also provides biosynthetic methods for production of caffeine, wherein the method comprises: providing guanine, a guanine deaminase, at least two methyl transferases selected from XMT, MXMT, and/or DXMT, and a methyl donor; contacting the guanine with the guanine deaminase to produce xanthine; contacting the xanthine with the XMT and a methyl donor, under conditions wherein the xanthine is methylated, to produce 7-methylxanthine; contacting the 7-methylxanthine with the MXMT and a methyl donor, under conditions wherein the 7-methylxanthine is methylated, to produce theobromine; and contacting the theobromine with the DXMT and a methyl donor, under conditions wherein the theobromine is methylated, to produce caffeine.

The present invention further provides biosynthetic methods for the production of caffeine comprising: providing guanine, a guanine deaminase, XMT, DXMT, and a methyl donor; contacting the guanine with the guanine deaminase to produce xanthine; contacting the xanthine with the DXMT and a methyl donor, under conditions wherein the xanthine is methylated, to produce caffeine.

In some embodiments of these biosynthetic methods for the production of caffeine, the guanine deaminase comprises a polypeptide selected from SEQ ID NOS:2, 4, 6, 8, 10, 12, 14, 16, 18, 20, and 22. In some embodiments, the guanine deaminase is encoded by a polynucleotide selected from SEQ ID NOS:1, 3, 5, 7, 9, 11, 13, 15, 17, 19, and 21.

In some embodiments of these biosynthetic methods for the production of caffeine, the methyl transferase comprises a polypeptide selected from SEQ ID NOS: 24, 26, 28, 30, 32, 34, 46, 38, 40, 42, 44, 46, 48, 50, 52, 54, 56, 58, 60, 62, 64, 66, 68, 70, 72, 74, 76, 78, 80, 82, or 84. In some embodiments, the methyl transferase is encoded by a polynucleotide selected from SEQ ID NOS:3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, and/or 83.

In some embodiments of these biosynthetic methods for the production of caffeine, the methyl donor is SAM. In some other embodiments, the methyl donor is an alternative methyl donor.

The present invention also provides non-naturally occurring polynucleotide sequences encoding a guanine deaminase, wherein the polynucleotide is codon-optimized and selected from SEQ ID NOS:2, 4, 6, 8, 10, 12, 14, 16, and 18.

The present invention also provides non-naturally occurring polynucleotide sequences encoding a methyl transferase, wherein the polynucleotide is codon-optimized and selected from SEQ ID NOS:24, 26, 28, 30, 32, 34, 46, 38, 40, 42, 44, 46, 48, 50, 52, 54, 56, 58, 60, 62, 64, 66, 68, 70, 72, 74, 76, 78, 80, and 82.

The present invention also provides expression vectors comprising at least one polynucleotide sequence selected from SEQ ID NOS:2, 4, 6, 8, 10, 12, 14, 16, and 18.

The present invention also provides expression vectors comprising at least one polynucleotide sequence selected from SEQ ID NOS:24, 26, 28, 30, 32, 34, 46, 38, 40, 42, 44, 46, 48, 50, 52, 54, 56, 58, 60, 62, 64, 66, 68, 70, 72, 74, 76, 78, 80, and 82.

The present invention also provides expression vectors comprising at least one polynucleotide sequence selected from SEQ ID NOS:2, 4, 6, 8, 10, 12, 14, 16, 18, and at least one polynucleotide sequence selected from SEQ ID NOS:24, 26, 28, 30, 32, 34, 46, 38, 40, 42, 44, 46, 48, 50, 52, 54, 56, 58, 60, 62, 64, 66, 68, 70, 72, 74, 76, 78, 80, and 82.

The present invention further provides host cells comprising at least one expression vector, wherein the expression vector comprises at least one polynucleotide sequence selected from SEQ ID NOS:2, 4, 6, 8, 10, 12, 14, 16, and 18.

In some further embodiments, the present invention further provides host cells comprising at least one expression vector, wherein the expression vector comprises at least one polynucleotide sequence selected from SEQ ID NOS:24, 26, 28, 30, 32, 34, 46, 38, 40, 42, 44, 46, 48, 50, 52, 54, 56, 58, 60, 62, 64, 66, 68, 70, 72, 74, 76, 78, 80, and 82.

In some further embodiments, the present invention further provides host cells comprising at least one expression vector, wherein the provides expression vector comprises at least one polynucleotide sequence selected from SEQ ID NOS:2, 4, 6, 8, 10, 12, 14, 16, 18, and at least one polynucleotide sequence selected from SEQ ID NOS:24, 26, 28, 30, 32, 34, 46, 38, 40, 42, 44, 46, 48, 50, 52, 54, 56, 58, 60, 62, 64, 66, 68, 70, 72, 74, 76, 78, 80, and 82.

The present invention also provides methods of expressing at least one non-naturally occurring polynucleotide selected from SEQ ID NOS:24, 26, 28, 30, 32, 34, 46, 38, 40, 42, 44, 46, 48, 50, 52, 54, 56, 58, 60, 62, 64, 66, 68, 70, 72, 74, 76, 78, 80, and 8; comprising placing the host cell comprising at least one polynucleotide sequence selected from SEQ ID NOS:24, 26, 28, 30, 32, 34, 46, 38, 40, 42, 44, 46, 48, 50, 52, 54, 56, 58, 60, 62, 64, 66, 68, 70, 72, 74, 76, 78, 80, and 82, in conditions suitable for the expression of the polynucleotide.

### BRIEF DESCRIPTION OF THE DRAWINGS

FIG. 1 provides schematics of the native and engineered pathways for production of caffeine.

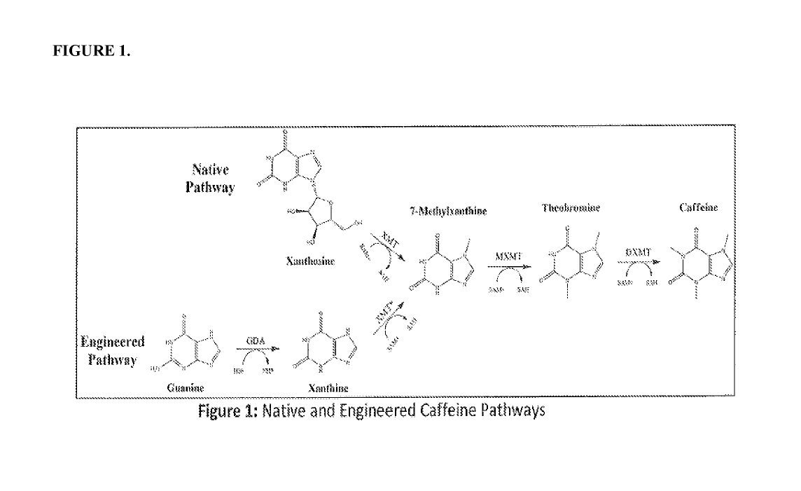

### DESCRIPTION OF THE INVENTION

The present invention provides an enzymatic means for the biosynthetic production of caffeine.

### Abbreviations and Definitions

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses. All patents, patent applications, articles and publications mentioned herein, both supra and infra, are hereby expressly incorporated herein by reference.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the application as a whole. All patents, patent applications, articles and publications mentioned herein, both supra and infra, are hereby expressly incorporated herein by reference.

Also, as used herein, the singular “a”, “an,” and “the” include the plural references, unless the context clearly indicates otherwise.

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

The term “about” means an acceptable error for a particular value. In some instances “about” means within 0.05%, 0.5%, 1.0%, or 2.0%, of a given value range. In some instances, “about” means within 1, 2, 3, or 4 standard deviations of a given value.

Furthermore, the headings provided herein are not limitations of the various aspects or embodiments of the invention which can be had by reference to the application as a whole. Accordingly, the terms defined immediately below are more fully defined by reference to the application as a whole. Nonetheless, in order to facilitate understanding of the invention, a number of terms are defined below.

Unless otherwise indicated, nucleic acids are written left to right in 5′ to 3′ orientation; amino acid sequences are written left to right in amino to carboxy orientation, respectively.

As used herein, the term “comprising” and its cognates are used in their inclusive sense (i.e., equivalent to the term “including” and its corresponding cognates).

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably herein to denote a polymer of at least two amino acids covalently linked by an amide bond, regardless of length or post-translational modification (e.g., glycosylation or phosphorylation).

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes.

The term “engineered,” “recombinant,” “non-naturally occurring,” and “variant,” when used with reference to a cell, a polynucleotide or a polypeptide refers to a material or a material corresponding to the natural or native form of the material that has been modified in a manner that would not otherwise exist in nature or is identical thereto but produced or derived from synthetic materials and/or by manipulation using recombinant techniques.

As used herein, “wild-type” and “naturally-occurring” refer to the form found in nature. For example a wild-type polypeptide or polynucleotide sequence is a sequence present in an organism that can be isolated from a source in nature and which has not been intentionally modified by human manipulation.

“Coding sequence” refers to that part of a nucleic acid (e.g., a gene) that encodes an amino acid sequence of a protein.

The term “percent (%) sequence identity” is used herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 2:482 [1981]), by the homology alignment algorithm of Needleman and Wunsch (Needleman and Wunsch, J. Mol. Biol., 48:443 [1970), by the search for similarity method of Pearson and Lipman (Pearson and Lipman, Proc. Natl. Acad. Sci. USA 85:2444 [1988]), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See, Altschul et al., J. Mol. Biol., 215: 403-410 [1990]; and Altschul et al., 1977, Nucleic Acids Res., 3389-3402 [1977], respectively). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (See, Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (See, Henikoff and Henikoff, Proc. Natl. Acad. Sci. USA 89:10915 [1989]). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

“Amino acid difference” or “residue difference” refers to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X3 as compared to SEQ ID NO:1” refers to a difference of the amino acid residue at the polypeptide position corresponding to position 3 of SEQ ID NO:1. Thus, if the reference polypeptide of SEQ ID NO:1 has a serine at position 3, then a “residue difference at position X3 as compared to SEQ ID NO:1” an amino acid substitution of any residue other than serine at the position of the polypeptide corresponding to position 3 of SEQ ID NO:1. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some instances (e.g., in Table 4.1), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X307H/X307P or X307H/P). The present application includes engineered polypeptide sequences comprising one or more amino acid differences that include either/or both conservative and non-conservative amino acid substitutions.

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g, proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered transaminase enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

A “functional fragment” or a “biologically active fragment” used interchangeably herein refers to a polypeptide that has an amino-terminal and/or carboxy-terminal deletion(s) and/or internal deletions, but where the remaining amino acid sequence is identical to the corresponding positions in the sequence to which it is being compared (e.g., a full-length methyltransferase or guanine deaminase of the present invention) and that retains substantially all of the activity of the full-length polypeptide.

“Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The recombinant methyltransferase or guanine deaminase polypeptides may be present within a cell, present in the cellular medium, or prepared in various forms, such as lysates or isolated preparations. As such, in some embodiments, the recombinant methyltransferase or guanine deaminase polypeptides can be an isolated polypeptide.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure methyltransferase or guanine deaminase composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant methyltransferase or guanine deaminase polypeptides are substantially pure polypeptide compositions.

“Improved enzyme property” refers to a polypeptide that exhibits an improvement in any enzyme property as compared to a reference polypeptide and/or as a wild-type polypeptide or another engineered polypeptide. Improved properties include but are not limited to such properties as increased protein expression, increased thermoactivity, increased thermostability, increased pH activity, increased stability, increased enzymatic activity, increased substrate specificity or affinity, increased specific activity, increased resistance to substrate or end-product inhibition, increased chemical stability, improved chemoselectivity, improved solvent stability, increased tolerance to acidic pH, increased tolerance to proteolytic activity (i.e., reduced sensitivity to proteolysis), and/or altered temperature profile.

“Increased enzymatic activity” or “enhanced catalytic activity” refers to an improved property of the engineered polypeptides, which can be represented by an increase in specific activity (e.g., product produced/time/weight protein) or an increase in percent conversion of the substrate to the product (e.g., percent conversion of starting amount of substrate to product in a specified time period using a specified amount of enzyme) as compared to the reference enzyme. Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity.

“Conversion” refers to the enzymatic conversion (or biotransformation) of a substrate(s) to the corresponding product(s). “Percent conversion” refers to the percent of the substrate that is converted to the product within a period of time under specified conditions. Thus, the “enzymatic activity” or “activity” of a polypeptide can be expressed as “percent conversion” of the substrate to the product in a specific period of time.

“Hybridization stringency” relates to hybridization conditions, such as washing conditions, in the hybridization of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tₘ as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Codon optimized” refers to changes in the codons of the polynucleotide encoding a protein to those preferentially used in a particular organism such that the encoded protein is more efficiently expressed in the organism of interest (i.e., the chosen host cell). Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the methyltransferase or guanine deaminase enzymes may be codon optimized for optimal production from the host organism selected for expression.

“Control sequence” refers herein to include all components, which are necessary or advantageous for the expression of a polynucleotide and/or polypeptide of the present application. Each control sequence may be native or foreign to the nucleic acid sequence encoding the polypeptide. Such control sequences include, but are not limited to, a leader, polyadenylation sequence, propeptide sequence, promoter sequence, signal peptide sequence, initiation sequence and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” refers to those conditions in the enzymatic conversion reaction solution (e.g., ranges of enzyme loading, substrate loading, temperature, pH, buffers, co-solvents, etc.) under which a polypeptide of the present application is capable of converting a substrate to the desired product compound, Exemplary “suitable reaction conditions” are provided in the present application and illustrated by the Examples. “Loading”, such as in “compound loading” or “enzyme loading” refers to the concentration or amount of a component in a reaction mixture at the start of the reaction. “Substrate” in the context of an enzymatic conversion reaction process refers to the compound or molecule acted on by the polypeptide. “Product” in the context of an enzymatic conversion process refers to the compound or molecule resulting from the action of the polypeptide on a substrate.

As used herein the term “culturing” refers to the growing of a population of microbial cells under any suitable conditions (e.g., using a liquid, gel or solid medium).

Recombinant polypeptides can be produced using any suitable methods known the art. Genes encoding the wild-type polypeptide of interest can be cloned in vectors, such as plasmids, and expressed in desired hosts, such as E. coli, etc. Variants of recombinant polypeptides can be generated by various methods known in the art. Indeed, there is a wide variety of different mutagenesis techniques well known to those skilled in the art. In addition, mutagenesis kits are also available from many commercial molecular biology suppliers. Methods are available to make specific substitutions at defined amino acids (site-directed), specific or random mutations in a localized region of the gene (regio-specific), or random mutagenesis over the entire gene (e.g., saturation mutagenesis). Numerous suitable methods are known to those in the art to generate enzyme variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. No. 6,117,679; U.S. Pat. No. 6,420,175; U.S. Pat. No. 6,376,246; U.S. Pat. No. 6,586,182; U.S. Pat. No. 7,747,391; U.S. Pat. No. 7,747,393; U.S. Pat. No. 7,783,428; and U.S. Pat. No. 8,383,346. After the variants are produced, they can be screened for any desired property (e.g., high or increased activity, or low or reduced activity, increased thermal activity, increased thermal stability, and/or acidic pH stability, etc.).

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature.

As used herein, the terms “host cell” and “host strain” refer to suitable hosts for expression vectors comprising DNA provided herein (e.g., the polynucleotides encoding methyltransferase or guanine deaminase). In some embodiments, the host cells are prokaryotic or eukaryotic cells that have been transformed or transfected with vectors constructed using recombinant DNA techniques as known in the art.

The term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide. In some embodiments, analogues means polypeptides that contain one or more non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

The term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

As used herein, “composition” and “formulation” encompass products comprising at least one enzyme of the present invention, intended for any suitable use (e.g., production of caffeine).

As used herein, “caffeine” refers to the xanthine alkaloid 1,3,7-trimethylxanthine.

As used herein, “xanthosine” refers to the nucleoside derived from xanthine and ribose 9-[(2R,3R,4S,5R)-3,4-dihydroxy-5-(hydroxymethyl)oxolan-2-yl]-3H-purine-2,6-dione.

As used herein, “xanthine” refers to the purine base 3,7-dihydropurine-2,6-dione.

As used herein, “7-methylxanthine” refers to the purine base 7-methyl-3H-purine-2,6-dione (also referred to as heteroxanthine, heteroxanthin, 7-methylxanthin, and 2,6-dihydroxy-7-methylpurine).

As used herein, “theobromine” refers to the purine base 3,7-dimethylpurine-2,6-dione (also referred to as 3,7-dimethylxanthine, diurobromine, teobromine, 83-67-0, theosalvose, and santheose).

As used herein, “guanine” refers to the purine base 2-amino-1H-purin-6(9H)-one.

As used herein, “trimethylglycine” and “betaine” refer to 2-trimethylammonioacetate.

As used herein, “butyrobetaine” refers to gamma-butyrobetaine or 3-carboxy-N,N,N-trimethyl-1-propanaminium.

As used herein, “guanine deaminase” and “GDA” refer to an enzyme that converts guanine to xanthine.

As used herein, “methyl group” refers to an alkyl functional group containing one carbon atom bonded to three hydrogen atoms.

As used herein, “methyltransferase” refers to an enzyme capable of catalyzing the transfer of a methyl group from a donor molecule to a specific substrate or group of substrates.

As used herein, “alternative methyl donor” refers to any methyl donor other than the preferred natural methyl donor for a specific enzyme. For example, for enzymes with the preferred natural methyl donor S-adenosylmethionine (SAM), an alternate methyl donor is any suitable methyl donor other than SAM.

As used herein, “XMT” refers to an enzyme that is capable of, but is not necessarily limited to, catalyzing the transfer of a methyl group to xanthosine to form 7-methylxanthosine. The designation can be based on measured activity or putative activity based on homology to proteins with measured activity.

As used herein, “MXMT” refers to an enzyme that is capable of, but is not necessarily limited to, catalyzing the transfer of a methyl group to 7-methylxanthine to form 3,7-dimethylxanthine (theobromine). The designation can be based on measured activity or putative activity based on homology to proteins with measured activity.

As used herein, “DXMT” refers to an enzyme that is capable of, but is not necessarily limited to, catalyzing the transfer of a methyl group to 3,7-dimethylxanthine (theobromine) to form 1,3,7-trimethylxanthine (caffeine). The designation can be based on measured activity or putative activity based on homology to proteins with measured activity.

### A New Biosynthetic Pathway for Caffeine Production:

The present invention provides a new biosynthetic pathway for the production of caffeine. In this pathway, guanine is used the starting material. Guanine deaminase (GDA) is used to convert guanine to xanthine. The xanthine is them converted to 7-methylxanthine by the enzyme XMT. Then, 7-methylxanthine is then methylated by MXMT to produce theobromine, which is then methylated by DXMT to produce caffeine. In some embodiments, native MXMT, DXMT and/or XMT enzymes are utilized, while in some other embodiments, recombinant enzymes find use. In some embodiments, caffeine is produced in a one-pot reaction, while in some other embodiments, the methods involve two-pot reactions.

### Polynucleotides Encoding Engineered Polypeptides, Expression Vectors and Host Cells:

The present invention provides polynucleotides encoding the polypeptides described herein. In some embodiments, the polynucleotides are codon-optimized for expression in the chosen host cells. In some embodiments, the polynucleotides are operatively linked to one or more heterologous regulatory sequences that control gene expression to create a recombinant polynucleotide capable of expressing the polypeptide. Expression constructs containing a heterologous polynucleotide encoding the polypeptides can be introduced into appropriate host cells to express the corresponding polypeptide.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the polypeptide. Thus, having knowledge of a particular amino acid sequence, those skilled in the art could make any number of different nucleic acids by simply modifying the sequence of one or more codons in a way which does not change the amino acid sequence of the protein. In this regard, the present invention specifically contemplates each and every possible variation of polynucleotides that could be made encoding the polypeptides described herein by selecting combinations based on the possible codon choices, and all such variations are to be considered specifically disclosed for any polypeptide described herein.

In various embodiments, the codons are preferably selected to fit the host cell in which the protein is being produced. For example, preferred codons used in bacteria are used for expression in bacteria. Consequently, codon optimized polynucleotides encoding the polypeptides contain preferred codons at about 40%, 50%, 60%, 70%, 80%, or greater than 90% of codon positions of the full length coding region.

In some embodiments, as described above, the polynucleotide encodes an engineered polypeptide having methyltransferase or guanine deaminase activity with the properties disclosed herein, wherein the polypeptide comprises an amino acid sequence having at least 80%, 85%, 86%, 87%, 88%, 89%, 90%, 91%, 92%, 93%, 94%, 95%, 96%, 97%, 98%, 99% or more identity to a reference sequence selected from the even-numbered sequences of SEQ ID NOS:1-84, or the amino acid sequence of any enzyme as disclosed in the Tables provided in the Examples.

In some embodiments, the polynucleotide encodes a polypeptide having guanine deaminase activity with the properties disclosed herein, wherein the polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NOS:2, 4, 6, 8, 10, 12, 14, 16, 18, 20, or 22.

In some embodiments, the polynucleotide encodes a polypeptide having methyltransferase activity with the properties disclosed herein, wherein the polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NOS:24, 26, 28, 30, 32, 34, 46, 38, 40, 42, 44, 46, 48, 50, 52, 54, 56, 58, 60, 62, 64, 66, 68, 70, 72, 74, 76, 78, 80, 82, or 84.

In some embodiments, an isolated polynucleotide encoding any of the methyltransferase or guanine deaminase polypeptides provided herein is manipulated in a variety of ways to provide for expression of the polypeptide. In some embodiments, the polynucleotides encoding the polypeptides are provided as expression vectors where one or more control sequences is present to regulate the expression of the polynucleotides and/or polypeptides. Manipulation of the isolated polynucleotide prior to its insertion into a vector may be desirable or necessary depending on the expression vector. The techniques for modifying polynucleotides and nucleic acid sequences utilizing recombinant DNA methods are well known in the art.

In some embodiments, the control sequences include among other sequences, promoters, leader sequences, polyadenylation sequences, propeptide sequences, signal peptide sequences, and transcription terminators. As known in the art, suitable promoters can be selected based on the host cells used. For bacterial host cells, suitable promoters for directing transcription of the nucleic acid constructs of the present application, include, but are not limited to the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA 75: 3727-3731 [1978]), as well as the tac promoter (See e.g., DeBoer et al., Proc. Natl Acad. Sci. USA 80: 21-25 [1983]). Exemplary promoters for filamentous fungal host cells, include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488 [1992]).

In some embodiments, the control sequence is a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice finds use in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra).

In some embodiments, the control sequence is a suitable leader sequence, a non-translated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells include, but are not limited to those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Bio., 15:5983-5990 [1995]).

In some embodiments, the control sequence is a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region that directs the expressed polypeptide into the secretory pathway of a host cell of choice finds use for expression of the methyltransferase or guanine deaminase polypeptides provided herein. Effective signal peptide coding regions for bacterial host cells include, but are not limited to the signal peptide coding regions obtained from the genes for Bacillus NC1B 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 57:109-137 [1993]). Effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase.

In some embodiments, the control sequence is a propeptide coding region that codes for an amino acid sequence positioned at the amino terminus of a polypeptide. The resultant polypeptide is referred to as a “proenzyme,” “propolypeptide,” or “zymogen,” in some cases). A propolypeptide can be converted to a mature active polypeptide by catalytic or autocatalytic cleavage of the propeptide from the propolypeptide. The propeptide coding region includes, but is not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (See e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

In another aspect, the present invention also provides a recombinant expression vector comprising a polynucleotide encoding a methyltransferase or guanine deaminase polypeptide, and one or more expression regulating regions such as a promoter and a terminator, a replication origin, etc., depending on the type of hosts into which they are to be introduced. in some embodiments, the various nucleic acid and control sequences described above are joined together to produce a recombinant expression vector which includes one or more convenient restriction sites to allow for insertion or substitution of the nucleic acid sequence encoding the polypeptide at such sites. Alternatively, the polynucleotide sequence(s) of the present invention are expressed by inserting the polynucleotide sequence or a nucleic acid construct comprising the polynucleotide sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), that can be conveniently subjected to recombinant DNA procedures and can result in the expression of the methyltransferase or guanine deaminase polynucleotide sequence. The choice of the vector will typically depend on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

In some embodiments, the expression vector preferably contains one or more selectable markers, which permit easy selection of transformed cells. A “selectable marker” is a gene the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in a filamentous fungal host cell include, but are not limited to, amdS (acetamidase), argB (ornithine carbamoyltransferases), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. In another aspect, the present invention provides a host cell comprising a polynucleotide encoding at least one methyltransferase or guanine deaminase polypeptide of the present application, the polynucleotide being operatively linked to one or more control sequences for expression of the methyltransferase or guanine deaminase enzyme(s) in the host cell. Host cells for use in expressing the polypeptides encoded by the expression vectors of the present invention are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae and Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Exemplary host cells are Escherichia coli strains (such as W3110 (AfhuA) and BL21).

Accordingly, in another aspect, the present invention provides methods for producing the methyltransferase or guanine deaminase polypeptides, where the methods comprise culturing a host cell capable of expressing a polynucleotide encoding the methyltransferase or guanine deaminase polypeptide under conditions suitable for expression of the polypeptide. In some embodiments, the methods further comprise the steps of isolating and/or purifying the methyltransferase or guanine deaminase polypeptides, as described herein.

Appropriate culture media and growth conditions for the above-described host cells are well known in the art. Polynucleotides for expression of the methyltransferase or guanine deaminase polypeptides may be introduced into cells by various methods known in the art. Techniques include, among others, electroporation, biolistic particle bombardment, liposome mediated transfection, calcium chloride transfection, and protoplast fusion.

The methyltransferase or guanine deaminase enzymes with the properties disclosed herein can be obtained by subjecting the polynucleotide encoding the naturally occurring or engineered methyltransferase or guanine deaminase polypeptide to mutagenesis and/or directed evolution methods known in the art, and as described herein. An exemplary directed evolution technique is mutagenesis and/or DNA shuffling (See e.g., Stemmer, Proc. Natl. Acad. Sci. USA 91:10747-10751 [1994]; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 16:258-261 [1998]), mutagenic PCR (See e.g., Caldwell et al., PCR Methods Appl., 3:S136-S140 [1994]), and cassette mutagenesis (See e.g., Black et al., Proc. Natl. Acad. Sci. USA 93:3525-3529 [1996]).

For example, mutagenesis and directed evolution methods can be readily applied to polynucleotides to generate variant libraries that can be expressed, screened, and assayed. Mutagenesis and directed evolution methods are well known in the art (See e.g., U.S. Pat. Nos. 5,605,793, 5,830,721, 6,132,970, 6,420,175, 6,277,638, 6,365,408, 6,602,986, 7,288,375, 6,287,861, 6,297,053, 6,576,467, 6,444,468, 5,811238, 6,117,679, 6,165,793, 6,180,406, 6,291,242, 6,995,017, 6,395,547, 6,506,602, 6,519,065, 6,506,603, 6,413,774, 6,573,098, 6,323,030, 6,344,356, 6,372,497, 7,868,138, 5,834,252, 5,928,905, 6,489,146, 6,096,548, 6,387,702, 6,391,552, 6,358,742, 6,482,647, 6,335,160, 6,653,072, 6,355,484, 6,03,344, 6,319,713, 6,613,514, 6,455,253, 6,579,678, 6,586,182, 6,406,855, 6,946,296, 7,534,564, 7,776,598, 5,837,458, 6,391,640, 6,309,883, 7,105,297, 7,795,030, 6,326,204, 6,251,674, 6,716,631, 6,528,311, 6,287,862, 6,335,198, 6,352,859, 6,379,964, 7,148,054, 7,629,170, 7,620,500, 6,365,377, 6,358,740, 6,406,910, 6,413,745, 6,436,675, 6,961,664, 7,430,477, 7,873,499, 7,702,464, 7,783,428, 7,747,391, 7,747,393, 7,751,986, 6,376,246, 6,426,224, 6,423,542, 6,479,652, 6,319,714, 6,521,453, 6,368,861, 7,421,347, 7,058,515, 7,024,312, 7,620,502, 7,853,410, 7,957,912, 7,904,249, and all related non-US counterparts; Ling et al., Anal. Biochem., 254:157-78 [1997]; Dale et al., Meth. Mol. Biol., 57:369-74 [1996]; Smith, Ann. Rev. Genet., 19:423-462 [1985]; Botstein et al., Science, 229:1193-1201 [1985]; Carter, Biochem. J., 237:1-7 [1986]; Kramer et al., Cell, 38:879-887 [1984]; Wells et al., Gene, 34:315-323 [1985]; Minshull et al., Curr. Op. Chem. Biol., 3:284-290 [1999]; Christians et al., Nat. Biotechnol., 17:259-264 [1999]; Crameri et al., Nature, 391:288-291 [1998]; Crameri, et al., Nat. Biotechnol., 15:436-438 [1997]; Zhang et al., Proc. Nat. Acad. Sci. U.S.A., 94:4504-4509 [1997]; Crameri et al., Nat. Biotechnol., 14:315-319 [1996]; Stemmer, Nature, 370:389-391 [1994]; Stemmer, Proc. Nat. Acad. Sci. USA, 91:10747-10751 [1994]; WO 95/22625; WO 97/0078; WO 97/35966; WO 98/27230; WO 00/42651; WO 01/75767; WO 2009/152336, and U.S. Pat. No. 6,537,746. all of which are incorporated herein by reference).

In some embodiments, the enzyme clones obtained following mutagenesis treatment are screened by subjecting the enzymes to a defined temperature (or other assay conditions) and measuring the amount of enzyme activity remaining after heat treatments or other assay conditions. Clones containing a polynucleotide encoding a methyltransferase or guanine deaminase polypeptide are then isolated from the gene, sequenced to identify the nucleotide sequence changes (if any), and used to express the enzyme in a host cell. Measuring enzyme activity from the expression libraries can be performed using any suitable method known in the art (e.g., standard biochemistry techniques, such as HPLC analysis).

For engineered polypeptides of known sequence, the polynucleotides encoding the enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides disclosed herein can be prepared by chemical synthesis using the classical phosphoramidite method (See e.g., Beaucage et al., Tetra. Lett., 22:1859-69 [1981]; and Matthes et al., EMBO J., 3:801-05 [1984]), as it is typically practiced in automated synthetic methods. According to the phosphoramidite method, oligonucleotides are synthesized (e.g., in an automatic DNA synthesizer), purified, annealed, ligated and cloned in appropriate vectors.

Accordingly, in some embodiments, a method for preparing the methyltransferase or guanine deaminase polypeptide can comprise: (a) synthesizing a polynucleotide encoding a polypeptide comprising an amino acid sequence selected from the amino acid sequence of any variant provided in the Tables in the Examples; and (b) expressing the methyltransferase or guanine deaminase polypeptide encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions.

The expressed methyltransferase or guanine deaminase polypeptide can be measured for any desired improved property (e.g., activity, selectivity, stability, etc.), using any suitable assay known in the art, including but not limited to the assays and conditions described herein.

In some embodiments, any of the methyltransferase or guanine deaminase polypeptides expressed in a host cell are recovered from the cells and/or the culture medium using any one or more of the well-known techniques for protein purification, including, among others, lysozyme treatment, sonication, filtration, salting-out, ultra-centrifugation, and chromatography.

Chromatographic techniques for isolation of the methyltransferase or guanine deaminase polypeptides include, among others, reverse phase chromatography high performance liquid chromatography, ion exchange chromatography, hydrophobic interaction chromatography, gel electrophoresis, and affinity chromatography. Conditions for purifying a particular enzyme depends, in part, on factors such as net charge, hydrophobicity, hydrophilicity, molecular weight, molecular shape, etc., and will be apparent to those having skill in the art. In some embodiments, affinity techniques may be used to isolate the methyltransferase or guanine deaminase enzymes. In some embodiments utilizing affinity chromatography purification, any antibody which specifically binds the methyltransferase or guanine deaminase polypeptide finds use. For the production of antibodies, various host animals, including but not limited to rabbits, mice, rats, etc., are immunized by injection with a methyltransferase or guanine deaminase polypeptide or a fragment thereof. In some embodiments, the methyltransferase or guanine deaminase polypeptide or fragment is attached to a suitable carrier, such as BSA, by means of a side chain functional group or linkers attached to a side chain functional group.

In some embodiments, the methyltransferase or guanine deaminase polypeptide is produced in a host cell by a method comprising culturing a host cell (e.g., an E. coli strain) comprising a polynucleotide sequence encoding a methyltransferase or guanine deaminase polypeptide as described herein under conditions conducive to the production of the methyltransferase or guanine deaminase polypeptide and recovering the polypeptide from the cells and/or culture medium.

In some embodiments, the invention encompasses a method of producing an methyltransferase or guanine deaminase polypeptide comprising culturing a recombinant bacterial cell comprising a polynucleotide sequence encoding a methyltransferase or guanine deaminase polypeptide wherein the polynucleotide sequence has at least 85%, 90%, 95%, 96%, 97%, 98%, 99%, or 100% sequence identity to at least one reference sequence selected from SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, and/or 83, under suitable culture conditions to allow the production of the methyltransferase or guanine deaminase polypeptide and optionally recovering the methyltransferase or guanine deaminase polypeptide from the culture and/or cultured cells (e.g., bacterial or fungal host cells).

In some embodiments, the invention encompasses a method of producing an methyltransferase or guanine deaminase polypeptide comprising culturing a recombinant bacterial cell comprising a polynucleotide sequence encoding a methyltransferase or guanine deaminase polypeptide having at least 85%, 90%, 95%, 96%, 97%, 98%, 99%, or 100% sequence identity to at least one reference sequence selected from SEQ ID NOS:2, 4, 6, 7, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 75, 78, 80, 82, and/or 84, under suitable culture conditions to allow the production of the methyltransferase or guanine deaminase polypeptide and optionally recovering the methyltransferase or guanine deaminase polypeptide from the culture and/or cultured cells (e.g., bacterial or fungal host cells).

In some embodiments, once the methyltransferase or guanine deaminase polypeptides are recovered from the recombinant host cells or cell culture and they are further purified by any suitable method(s) known in the art. In some additional embodiments, the purified methyltransferase or guanine deaminase polypeptides are combined with other ingredients and compounds to provide compositions and formulations comprising the methyltransferase or guanine deaminase polypeptide as appropriate for different applications and uses (e.g., pharmaceutical compositions).

### Compositions:

The present invention provides compositions comprising the enzymes provided herein, as well as compositions comprising the end-product, caffeine. In some embodiments, the compositions comprise food, while in other embodiments, the compositions comprise beverages.

The foregoing and other aspects of the invention may be better understood in connection with the following non-limiting examples. The examples are provided for illustrative purposes only and are not intended to limit the scope of the present invention in any way.

### EXPERIMENTAL

The following Examples, including experiments and results achieved, are provided for illustrative purposes only and are not to be construed as limiting the present invention.

In the experimental disclosure below, the following abbreviations apply: ppm (parts per million); M (molar); mM (millimolar), uM and μM (micromolar); nM (nanomolar); mol (moles); gm and g (gram); mg (milligrams); ug and μg (micrograms); L and l (liter); ml and mL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); ° C. (degrees Centigrade); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, Conn.); HPLC (high pressure liquid chromatography); LC (liquid chromatography); SDS-PAGE (sodium dodecyl sulfate polyacrylamide gel electrophoresis); FIOPC (fold improvements over positive control); LB (Luria broth); TB (Terrific broth); MeOH (methanol); (IPTG) isopropyl-β-D-thiogalactoside; Athens Research (Athens Research Technology, Athens, Ga.); ProSpec (ProSpec Tany Technogene, East Brunswick, N.J.); Sigma-Aldrich (Sigma-Aldrich, St. Louis, Mo.); Ram Scientific (Ram Scientific, Inc., Yonkers, N.Y.); Pall Corp. (Pall, Corp., Pt. Washington, N.Y.); Millipore (Millipore, Corp., Billerica Mass.); Difco (Difco Laboratories, BD Diagnostic Systems, Detroit, Mich.); Molecular Devices (Molecular Devices, LLC, Sunnyvale, Calif.); Kuhner (Adolf Kuhner, AG, Basel, Switzerland); Cambridge Isotope Laboratories, (Cambridge Isotope Laboratories, Inc., Tewksbury, Mass.); Applied Biosystems (Applied Biosystems, part of Life Technologies, Corp., Grand Island, N.Y.), Agilent (Agilent Technologies, Inc., Santa Clara, Calif.); Thermo Scientific (part of Thermo Fisher Scientific, Waltham, Mass.); Corning (Corning, Inc., Palo Alto, Calif.); Megazyme (Megazyme International, Wicklow, Ireland); Enzo (Enzo Life Sciences, Inc., Farmingdale, N.Y.); GE Healthcare (GE Healthcare Bio-Sciences, Piscataway, N.J.); Pierce (Pierce Biotechnology (now part of Thermo Fisher Scientific), Rockford, Ill.); Phenomenex (Phenomenex, Inc., Torrance, Calif.); Optimal (Optimal Biotech Group, Belmont, Calif.); and Bio-Rad (Bio-Rad Laboratories, Hercules, Calif.).

The following polynucleotide and polypeptide sequences find use in the present invention. In some cases (as shown below), the polynucleotide sequence is followed by the encoded polypeptide.

GDA_01:
(SEQ ID NO: 1)
ATGATGTCCGGCGAACATACTCTGAAGGCAGTACGCGGGTCATTCATCGATGTTACCCGT
ACTATTGATAATCCAGAAGAAATAGCCTCAGCTCTGCGGTTCATCGAGGATGGATTGTTA
TTAATAAAGCAGGGTAAAGTTGAGTGGTTCGGCGAGTGGGAAAACGGTAAGCACCAGAT
TCCGGATACTATTCGTGTTCGTGACTATCGGGGGAAACTTATAGTTCCCGGGTTCGTGGA
TACGCACATCCATTACCCCCAGTCCGAAATGGTAGGAGCGTATGGCGAACAATTATTGG
AATGGCTCAACAAGCATACATTCCCGACTGAGCGTAGATATGAAGATCTGGAATATGCC
CGCGAAATGAGTGCGTTTTTCATAAAGCAGCTTTTGCGGAATGGCACTACAACCGCTCTT
GTCTTTGGTACCGTGCATCCTCAGTCTGTCGATGCTCTGTTTGAGGCCGCTTCACACATCA
ATATGCGGATGATAGCAGGAAAGGTAATGATGGATAGAAACGCGCCCGATTACCTCCTC
GATACTGCCGAATCAAGCTATCATCAAAGCAAGGAGCTGATCGAGCGTTGGCATAAAAA
TGGACGCCTTCTTTATGCGATTACCCCTAGATTCGCACCAACGAGCTCACCTGAACAGAT
GGCCATGGCACAGCGCTTAAAGGAAGAGTATCCTGATACATGGGTCCACACGCACCTTT
GTGAGAATAAGGATGAAATTGCATGGGTCAAATCACTGTACCCAGATCATGATGGCTAC
TTAGATGTGTATCACCAATATGGGTTAACTGGTAAGAATTGCGTATTCGCTCACTGTGTG
CATCTTGAAGAAAAGGAATGGGATCGTCTGTCAGAGACTAAATCGTCAATTGCATTCTGT
CCCACATCCAATCTGTATCTCGGTAGCGGCCTGTTTAATCTGAAAAAGGCGTGGCAAAAG
AAGGTCAAAGTCGGGATGGGTACAGACATAGGCGCAGGTACAACGTTTAATATGTTGCA
GACGTTGAACGAGGCCTACAAGGTCTTGCAATTGCAAGGCTATCGGCTTTCAGCTTACGA
AGCTTTTTATCTGGCGACTTTGGGCGGAGCAAAATCCCTGGGTTTAGATGATCTGATAGG
GAACTTCCTGCCTGGTAAAGAGGCTGACTTCGTAGTTATGGAACCCACTGCCACTCCACT
TCAACAACTTCGTTATGACAATTCGGTTAGTTTGGTAGACAAACTTTTCGTTATGATGAC
GTTAGGGGACGACCGCAGTATCTATAGAACCTACGTTGATGGTAGACTGGTATATGAGC
GGAATTAA
Polypeptide sequence encoded by the polynucleotide of SEQ ID NO: 1:
(SEQ ID NO: 2)
MMSGEHTLKAVRGSFIDVTRTIDNPEEIASALRFIEDGLLLIKQGKVEWFGEWENGKHQIPDTI
RVRDYRGKLIVPGFVDTHIHYPQSEMVGAYGEQLLEWLNKHTFPTERRYEDLEYAREMSAF
FIKQLLRNGTTTALVFGTVHPQSVDALFEAASHINMRMIAGKVMMDRNAPDYLLDTAESSY
HQSKELIERWHKNGRLLYAITPRFAPTSSPEQMAMAQRLKEEYPDTWVHTHLCENKDEIAW
VKSLYPDHDGYLDVYHQYGLTGKNCVFAHCVHLEEKEWDRLSETKSSIAFCPTSNLYLGSG
LFNLKKAWQKKVKVGMGTDIGAGTTFNMLQTLNEAYKVLQLQGYRLSAYEAFYLATLGGA
KSLGLDDLIGNFLPGKEADFVVMEPTATPLQQLRYDNSVSLVDKLFVMMTLGDDRSIYRTYV
DGRLVYERN
GDA_02:
(SEQ ID NO: 3)
ATGAATCACGAAACATTCCTCAAAAGAGCCGTCACGCTTGCTTGCGAGGGTGTTAATGCC
GGCATTGGGGGACCATTTGGCGCAGTCATCGTTAAAGATGGGGCGATCATAGCTGAGGG
CCAGAACAACGTGACTACCTCTAATGACCCAACAGCACACGCGGAGGTGACAGCTATAC
GGAAGGCTTGCAAAGTACTCGGTGCGTACCAACTGGACGACTGTATTTTATATACTTCCT
GTGAGCCATGCCCAATGTGCCTCGGTGCGATATACTGGGCTAGACCAAAAGCTGTCTTTT
ACGCCGCCGAACATACCGATGCAGCGGAAGCGGGGTTCGATGACTCCTTCATATACAAG
GAAATCGACAAACCGGCCGAGGAGCGCACAATTCCCTTCTACCAGGTCACGCTGACCGA
GCACCTTTCTCCTTTCCAAGCCTGGAGAAACTTCGCTAACAAGAAAGAGTATTAA
Polypeptide sequence encoded by the polynucleotide of SEQ ID NO: 3:
(SEQ ID NO: 4)
MNHETFLKRAVTLACEGVNAGIGGPFGAVIVKDGAIIAEGQNNVTTSNDPTAHAEVTAIRKA
CKVLGAYQLDDCILYTSCEPCPMCLGAIYWARPKAVFYAAEHTDAAEAGFDDSFIYKEIDKP
AEERTIPFYQVTLTEHLSPFQAWRNFANKKEY
GDA_03:
(SEQ ID NO: 5)
ATGATGGACTACCAGACCGCGGTGCGCGGTGCGTTTTTCGATATTGCCGGCGTAGCAGAA
ACCCCCGATGAAGTGGCAGCACAGGCCCGTTACCTCGATGATGGGTTACTTTTTCTGCGC
GATGGAAAGATAATTGCGTTGCTGCCGTGGCAAGAAGGGGAAGCATTCTTACATCCGCT
GAAGGGCTATGTAGATCAGCGCGGCAAGCTGTTACTGCCTGGATTTGTCGATACACATAT
CCACTACCCTCAGACCGAAATGATAGGTGCCTTTGGTGAGCAGCTCCTTGAATGGCTTAC
AACATATACGTTTCCTGTCGAATCACAGTTTGCCGATGCCGATTATGCACAGGAAATCGC
GCAGTTCTTTGTCAACCAGTTAATTTCACACGGCACTACAACCGCACTGGTCTTTTGCAC
ACTGCACCCGGCCTCTGCCGAGGCCTTATTCTCGGAAGCATTACGGTTGAACATGAGACT
GATAGCCGGTAAGGTCATGATGGATCGCCATGTCCCAGATTACTTGTGCGAAACGGCCG
GTGAATCATACGAACAAACAAGAGAGTTGATACTTCGGTGGCATCAGCGTGGACGGTTA
GGTTACGCCATCACACCTCGCTTTGCGCCAACTTCAACTCCTGCCCTTTTAGAAGCGGTCC
AGAGACTTCGTACCGAATTTCCTGATACCTGGTTGCAGACCCACTTATCAGAAAACCGCG
AAGAAATTGCTTGGGTGAAACAGCTGTGGCCAGAGCATGAGCATTATTTGGACGTCTATC
ACCATTATCAGCTGACAGGGGAGCGGTCAGTCTTCGCGCACGGCATTCATTTGGATGATG
CGGAGTGGCAGTGTCTGCACGACACTGGGTCTGCCGTCGCATTTTGTCCGACATCAAATC
TGTTCCTGGGATCTGGTCTGTTTCGCTTACCCGCGTGTTGGCAGCATCAGGTAAGAATGG
GAATAGGTAGTGACGTTGGTGCTGGAACTACATTCTCTATGCTCCGTACACTGGGTGAAG
CGTATAAAGTCGGACAGCTGCAGTCCTACCGGTTGCGTGCATCGGAAGCCTTCTACCATG
CTACGTTGGGAGGGGCTAGAGCATTAAGATTGGAGGATAAAATTGGTAACTTCCAGCCG
GGTAAAGAGGCGGATTTTGTCGTAATTGATCCCGCTGTTACACCTCTTCAACGTCTGCGT
ACCGGACGCTGCCATGACATCTATGAACAGCTTTTCGTTTTAATGACACTGGGGGACGAG
AGAAACATCAGTGAAACATGGGTCAACGGGGAGCGCGTTTGGTGCCAGGATTAA
Polypeptide sequence encoded by the polynucleotide of SEQ ID NO: 5:
(SEQ ID NO: 6)
MMDYQTAVRGAFFDIAGVAETPDEVAAQARYLDDGLLFLRDGKIIALLPWQEGEAFLHPLK
GYVDQRGKLLLPGFVDTHIHYPQTEMIGAFGEQLLEWLTTYTFPVESQFADADYAQEIAQFF
VNQLISHGTTTALVFCTLHPASAEALFSEALRLNMRLIAGKVMMDRHVPDYLCETAGESYEQ
TRELILRWHQRGRLGYAITPRFAPTSTPALLEAVQRLRTEFPDTWLQTHLSENREEIAWVKQL
WPEHEHYLDVYHHYQLTGERSVFAHGIHLDDAEWQCLHDTGSAVAFCPTSNLFLGSGLFRL
PACWQHQVRMGIGSDVGAGTTFSMLRTLGEAYKVGQLQSYRLRASEAFYHATLGGARALR
LEDKIGNFQPGKEADFVVIDPAVTPLQRLRTGRCHDIYEQLFVLMTLGDERNISETWVNGER
VWCQD
GDA_04:
(SEQ ID NO: 7)
ATGACTGTCACTCGCAAGGCTTATCGCGCGGCAATACTTCACTCAATCGCTGATCCCGCA
GAGGTCGGATTAGACGCGTCACATGAGTACTTCGAGGACGGCCTTCTTGTGATCGACGG
AGGTCGTATTCAAGCGGTTGGCCATGCGAGTGACCTGCTGCCCACCCTGGATGCTGATAT
CCCGGTTGAACATTATCAGGATGCTCTCATTACGCCTGGCTTTATAGATACTCATATCCAC
TTCCCCCAAACGGGTATGATAGGCAGTTATGGGGAACAGTTGCTGGATTGGCTGAACAC
CTACACATTTCCGTGCGAGAAACAGTTTGCGGATAAAGATCATGCAGATCAAGTGGCCC
ATGTGTTCTTAAAAGAGCTGTTACGCAACGGCACCACGACCGCGCTGGTATTTGGTTCGG
TACATCCCGAAAGCGTAAATGCGCTGTTTGAGGCAGCTGAGCGTCTTGATCTTCGGCTCA
TAGCTGGTAAAGTTATGATGGATCGCAACGCACCCGATTATCTGACCGACACTGCTGAGA
GCAGCTACGCCCAGTCCAAAGCTCTCATCGAGCGGTGGCACGGTAAAGGCAGATTACAC
TACGCCGTGACGCCGAGATTTGCGCCAACATCTACGCCAGAACAGCTCGCACGTGCAGG
TCAACTGCTGAAAGAGCACCCTGGAGTGTATTTGCATACCCATCTCAGCGAGAACCTCCA
AGAAATCGACTGGGTTAAGTCTTTATTCCCTGAACAAAAGGGCTACCTTGATGTCTATGA
CCACTTCGAACTGTTAGGGGAACGCAGCGTATTTGCTCACGGGGTTCATCTCTGCGATGA
AGAATGTCAGCGTTTGGCGGAAACAGGTTCGGCTGTGGCATTCTGCCCTACGTCCAATTT
ATTTCTCGGAAGTGGACTGTTTAATCTTCCCCAGGCCGAGAGATTCAAAGTGAACGTGGG
GCTGGGGACAGATGTGGGTGCTGGTACTAGTTTCTCCCTGCTGAATACCCTGAACGAAGC
TTATAAGGTTATGCAGCTGCAAGGTGCGCGGCTCCACCCGTACAAATCTCTTTACCTCGC
TACACTTGGTGGTGCAAGAGCGTTAAGACTTGATGACCGCATTGGGAGCCTGCGCCCTGG
TAATGATGCAGACTTTGTGGTGCTCGATTATAAAGCTACTCCCTTACTGGACTATCGGTT
ACAGCAATCTAGATCCATCGAGGAAACATTATTTGTGCTGACAACTCTCGGGGATGATAG
AACTGTTCGCGAAACGTATGCGGCAGGTCGGTGTGTCCATCAAAGAGAAGGATAA
Polypeptide sequence encoded by the polynucleotide of SEQ ID NO: 7:
(SEQ ID NO: 8)
MTVTRKAYRAAILHSIADPAEVGLDASHEYFEDGLLVIDGGRIQAVGHASDLLPTLDADIPVE
HYQDALITPGFIDTHIHFPQTGMIGSYGEQLLDWLNTYTFPCEKQFADKDHADQVAHVFLKE
LLRNGTTTALVFGSVHPESVNALFEAAERLDLRLIAGKVMMDRNAPDYLTDTAESSYAQSK
ALIERWHGKGRLHYAVTPRFAPTSTPEQLARAGQLLKEHPGVYLHTHLSENLQEIDWVKSLF
PEQKGYLDVYDHFELLGERSVFAHGVHLCDEECQRLAETGSAVAFCPTSNLFLGSGLFNLPQ
AERFKVNVGLGTDVGAGTSFSLLNTLNEAYKVMQLQGARLHPYKSLYLATLGGARALRLD
DRIGSLRPGNDADFVVLDYKATPLLDYRLQQSRSIEETLFVLTTLGDDRTVRETYAAGRCVH
QREG
GDA_05:
(SEQ ID NO: 9)
ATGACCAAATCGGATTTATTGTTCGATAAATTTAACGATAAACACGGAAAATTTTTGGTA
TTCTTTGGAACATTCGTGGACACGCCGAAGCTGGGAGAACTCCGGATTCGCGAAAAGAC
GTCAGTGGGAGTTTTAAACGGAATTATACGGTTCGTTAACCGGAACTCGTTAGATCCGGT
TAAGGATTGCCTGGATCATGATTCTTCGTTGTCTCCGGAGGACGTCACCGTCGTCGATAT
AATCGGTAAAGATAAGACTCGGAACAACTCCTTCTACTTTCCCGGATTTGTCGACACTCA
TAATCATGTTTCACAGTACCCAAATGTGGGGGTTTTTGGTAATTCAACACTGTTGGATTG
GCTGGAGAAATATACTTTCCCGATCGAGGCTGCTTTGGCCAACGAAAACATTGCCCGTGA
GGTCTACAACAAGGTTATCTCCAAAACTCTGAGTCATGGTACAACTACTGTGGCGTATTA
CAACACCATAGATCTGAAGTCTACAAAGCTTCTGGCCCAGTTATCATCCCTTCTCGGTCA
GCGTGTACTTGTTGGCAAAGTCTGTATGGATACCAATGGACCGGAATATTATATTGAAGA
TACGAAAACGAGCTTCGAATCTACGGTCAAAGTTGTCAAATACATACGCGAAACGATTT
GCGATCCTCTGGTAAACCCAATAGTGACTCCTAGATTCGCTCCATCGTGCAGCCGTGAGT
TAATGCAGCAGTTAAGCAAGCTTGTTAAGGACGAAAATATCCATGTTCAGACGCACCTGT
CAGAGAACAAAGAAGAAATCCAATGGGTGCAGGATCTGTTCCCCGAGTGCGAATCTTAT
ACAGACGTCTATGATAAGTATGGCCTGCTCACTGAGAAAACGGTTTTAGCACATTGCATT
CATCTGACAGATGCAGAAGCACGTGTTATAAAGCAGCGGCGCTGTGGCATCAGTCACTG
CCCGATAAGTAACTCCTCATTGACATCGGGAGAATGTCGGGTTCGCTGGCTGCTGGATCA
AGGTATTAAAGTTGGTCTGGGAACGGATGTCTCGGCAGGGCATTCATGTTCAATTTTGAC
AACTGGACGGCAGGCGTTCGCGGTTAGCCGCCATTTAGCCATGCGTGAGACTGATCACG
CAAAATTATCGGTTTCAGAGTGCTTGTTTCTTGCAACTATGGGCGGTGCGCAGGTTTTAA
GAATGGATGAGACGTTGGGAACATTTGATGTCGGTAAACAATTCGATGCGCAAATGATA
GATACGAATGCTCCGGGCTCCAACGTAGATATGTTTCACTGGCAGTTGAAGGAAAAAGA
TCAAATGCAGGAGCAAGAGCAGGAACAAGGACAGGACCCTTACAAAAATCCGCCGTTAC
TGACAAACGAAGATATCATTGCGAAGTGGTTCTTTAATGGTGACGATCGTAACACCACA
AAAGTGTGGGTGGCAGGTCAGCAAGTTTATCAGATCTAA
Polypeptide sequence encoded by the polynucleotide of SEQ ID NO: 9:
(SEQ ID NO: 10)
MTKSDLLFDKFNDKHGKFLVFFGTFVDTPKLGELRIREKTSVGVLNGIIRFVNRNSLDPVKDC
LDHDSSLSPEDVTVVDIIGKDKTRNNSFYFPGFVDTHNHVSQYPNVGVFGNSTLLDWLEKYT
FPIEAALANENIAREVYNKVISKTLSHGTTTVAYYNTIDLKSTKLLAQLSSLLGQRVLVGKVC
MDTNGPEYYIEDTKTSFESTVKVVKYIRETICDPLVNPIVTPRFAPSCSRELMQQLSKLVKDEN
IHVQTHLSENKEEIQWVQDLFPECESYTDVYDKYGLLTEKTVLAHCIHLTDAEARVIKQRRC
GISHCPISNSSLTSGECRVRWLLDQGIKVGLGTDVSAGHSCSILTTGRQAFAVSRHLAMRETD
HAKLSVSECLFLATMGGAQVLRMDETLGTFDVGKQFDAQMIDTNAPGSNVDMFHWQLKEK
DQMQEQEQEQGQDPYKNPPLLTNEDIIAKWFFNGDDRNTTKVWVAGQQVYQI
GDA_06:
(SEQ ID NO: 11)
ATGCGGGATTTGGAGAAGAACATAAAAATTCTGAAGGGCAACATCATCTACACCGAAAA
CTCTGATTCATTTAACGTTGTGGAAAAGGGGTTTATCATTGTAAAGGATGGCATTATCGA
TGGCACGTACGAGAAATTACCTGAAGAATTTAAAGATGTTCAAGTCGAGGATTACGGTG
ATAAACTGATTATACCTGGGTTTATCGACCTTCACACCCATGCATCTCAGTTCGCTATCAA
GGGTATTGGATATGATAAAGAGCTTTTGCCATGGTTGGAAACTTACACATTTCCTGAGGA
GGCGAAGTTTATCGACAAGGTGTATGCCGAAAAAGTATATAAGGAGTTTGTGGACGAAT
TGTATGAGGAGGGCACTACCCGTGCAGTAATTTTTGCTACAATACATAGCGAATCTACAG
AGATCCTGATGAAATTACTGGAAGAAAAAGGCATTGAGGCTTATGTTGGGAAAGTCAAT
ATGGATCGCAATTGCCCTGAATCCCTCAAAGAAAATTCAGACGAGTCTGTGGAGGCGAC
CATCAAGTGGATTGAGAACTCAAGCAAGAAATATAAGTATGTTAAACCAATCATCACCC
CAAGATTCGTACCTTCATGTACAGGCCACTTAATGAAAGAACTGGGCAGCATCGCAGTTT
CGAAATCGATGCCGGTCCAGAGCCATTTGTCCGAGAACCTTAGTGAAATAGAGTGGGTT
AAAGAATTGCACCCCGAATGTAAGAACTACGGCGAAGTGTATGACAAGTTCAACTTGTT
TGGCCAAACCAAAACAATTATGGCGCACTGCGTGTATTTAACAGAGGATGAGATCAACG
CTATAGAAGATAATAACGTGACTATTGCTCACTGCCCAACCAGCAACGTTAATATCTCGT
CGGGAATTGCTCCGATCAATACGTTGCTGAGAAAGAAGAAAGTGAAAATTGGACTCGGC
TCCGATATTGCCGGAGGGGAAAGTCTGAGTCTTCTTTCTGTGATGAAAAGTGCGGTTTCT
TTATCGAAAATGCGCGCGGTGTGTTTCAAAGAAGAAGAAAAGGCGCTGACGCTCCCCGA
AGTGTTTTACTTAGCGACTAAAGGTGGCGGTTCCTTCTTTGGTAATGTAGGCTCGTTTGAG
AAATCTTACGAGTTTGATGCCCTGATTATCGACGACGACTCTTTATGGAAAGTTAATAAA
GGAAATATTGAGGAACGTCTGGAGAAACTGGTGTACTTAGGAGACAAACGCAATATTAT
AAAACGCTATGTCTGTGGACGCGAAATCTAA
Polypeptide sequence encoded by the polynucleotide of SEQ ID NO: 11:
(SEQ ID NO: 12)
MRDLEKNIKILKGNIIYTENSDSFNVVEKGFIIVKDGIIDGTYEKLPEEFKDVQVEDYGDKLIIP
GFIDLHTHASQFAIKGIGYDKELLPWLETYTFPEEAKFIDKVYAEKVYKEFVDELYEEGTTRA
VIFATIHSESTEILMKLLEEKGIEAYVGKVNMDRNCPESLKENSDESVEATIKWIENSSKKYKY
VKPIITPRFVPSCTGHLMKELGSIAVSKSMPVQSHLSENLSEIEWVKELHPECKNYGEVYDKF
NLFGQTKTIMAHCVYLTEDEINAIEDNNVTIAHCPTSNVNISSGIAPINTLLRKKKVKIGLGSDI
AGGESLSLLSVMKSAVSLSKMRAVCFKEEEKALTLPEVFYLATKGGGSFFGNVGSFEKSYEF
DALIIDDDSLWKVNKGNIEERLEKLVYLGDKRNIIKRYVCGREI
GDA_07:
(SEQ ID NO: 13)
ATGTCAGCTCACAGTGCACAACAGCGGGATGAGTATCTTAACCTTGCCATACAGCTGGCG
GTGCACAATGTCAGCGACGGCGGTGGACCTTTTGGGGCAGTAGTAGTGACTGCTGACGG
AACAGTGCATGAGGGAGTCAACCGGGTGACACGCGATCATGACCCAACGGCACATGCTG
AAGTGGTCGCCATACGTCGCGCAGCTGCAGCGTCGAAACGGTTCGACCTGACTGGGTCT
GTCTTATATGCGTCGTGCGAACCGTGTCCCCTGTGCCTGTCTGCTACGTTGTGGGCTCGCA
TTGGGCATGTCTACTTCGCAGCGGATAGACATGGAGCAGCAAAGGCCGGGTTCGACGAT
GCGGTGTTTTATGAATATTTCGCAGGCACACGGCCCGAGTTACTTCCCGTTGAACATGCA
GAGCTTGCTGCCAGTAACGAACCATTTGACGCATGGAGAAACCACGCTCGCCGTACAGC
CTATTAA
Polypeptide sequence encoded by the polynucleotide of SEQ ID NO: 13:
(SEQ ID NO: 14)
MSAHSAQQRDEYLNLAIQLAVHNVSDGGGPFGAVVVTADGTVHEGVNRVTRDHDPTAHAE
VVAIRRAAAASKRFDLTGSVLYASCEPCPLCLSATLWARIGHVYFAADRHGAAKAGFDDAV
FYEYFAGTRPELLPVEHAELAASNEPFDAWRNHARRTAY
GDA_08:
(SEQ ID NO: 15)
ATGACGACTGTAGGCATCCGGGGCACTTTCTTTGACTTTGTGGATGATCCATGGAAGCAC
ATAGGTAACGAGCAAGCGGCAGCGAGATTCCACCAGGACGGGCTCATGGTAGTCACAGG
TGGCGTGATAAAAGCTTTTGGGCCACACGACAAAATCGCTGCGGCGCATCCCGGGGTTG
AGATTACACACATCAAGGACCGGATAATCGTTCCGGGTTTTATTGATGGTCACATTCATC
TGCCGCAAACTCGTGTCCTTGGTGCGTATGGTGAGCAGTTGCTTCCGTGGCTGCAGAAAA
GTATCTACCCCGAAGAGATCAAATACAAAGATCGTAACTACGCTCGCGAGGGCGTGAAA
CGTTTCTTAGATGCGCTGCTGGCAGCTGGTACTACGACTTGTCAGGCTTTTACGAGCTCCT
CTCCTGTTGCGACTGAAGAGCTTTTTGAAGAAGCAGCACGGCGGAATATGCGTGTTATCG
CGGGTCTGACGGGCATCGATCGCAATGCTCCTGCTGAATTCATAGATACTCCAGAAAACT
TCTACCGCGATAGTAAACGGCTCATTGCGCAGTATCATAACAAAGGGAGAAACTTATAC
GCGATCACACCCCGTTTCGCATTTGGAGCGAGCCCCGAACTGCTCAAAGCATGCCAACGT
TTAAAACACGAGCATCCTGATTGCTGGGTCAATACCCATATTTCTGAGAATCCCGCTGAG
TGCTCAGGGGTTCTGGTTGAGCATCCGGATTGTCAGGACTACCTGGGTGTTTACGAGAAG
TTTGATCTCGTAGGACCAAAATTTAGCGGAGGACACGGGGTATATCTGTCGAATAACGA
GTTCCATAGAATGTCTAAGAAGGGTGCGGCTGTGGTATTTTGCCCTTGCTCCAATTTGTTT
CTTGGAAGCGGTTTGTTTCGGCTCGGTAGAGCCACCGATCCAGAACATCGGGTCAAAATG
AGTTTCGGGACCGATGTGGGCGGCGGTAATCGCTTTTCTATGATTTCCGTTTTGGACGAC
GCCTATAAAGTAGGTATGTGCAACAACACTATGCTGGATGGTTCGATAGATCCCGCACG
GAAAGATCTGGCTGAGGCAGAGCGTAATAAATTATCCCCATACCGCGGTTTTTGGTCAGT
TACCTTAGGGGGTGCAGAAGGCCTTTACATTGACGATAAATTAGGCAATTTTGAACCAGG
TAAAGAAGCAGATTTTGTAGCGTTAGATCCGAACGGTGGACAGCTTGCTCAGCCATGGC
ACCAAAGTCTTATAGCTGACGGAGCGGGACCGCGTACCGTAGATGACGCAGCCAACATG
CTGTTCTCCGTAATGATGGTTGGGGATGATCGTTGTGTGGACGAGACGTGGGTCATGGGA
AAACGGCTGTACAAACAGTCATAA
Polypeptide sequence encoded by the polynucleotide of SEQ ID NO: 15:
(SEQ ID NO: 16)
MTTVGIRGTFFDFVDDPWKHIGNEQAAARFHQDGLMVVTGGVIKAFGPHDKIAAAHPGVEI
THIKDRIIVPGFIDGHIHLPQTRVLGAYGEQLLPWLQKSIYPEEIKYKDRNYAREGVKRFLDAL
LAAGTTTCQAFTSSSPVATEELFEEAARRNMRVIAGLTGIDRNAPAEFIDTPENFYRDSKRLIA
QYHNKGRNLYAITPRFAFGASPELLKACQRLKHEHPDCWVNTHISENPAECSGVLVEHPDCQ
DYLGVYEKFDLVGPKFSGGHGVYLSNNEFHRMSKKGAAVVFCPCSNLFLGSGLFRLGRATD
PEHRVKMSFGTDVGGGNRFSMISVLDDAYKVGMCNNTMLDGSIDPARKDLAEAERNKLSPY
RGFWSVTLGGAEGLYIDDKLGNFEPGKEADFVALDPNGGQLAQPWHQSLIADGAGPRTVDD
AANMLFSVMMVGDDRCVDETWVMGKRLYKQS
GDA_09:
(SEQ ID NO: 17)
ATGAAGATTTACCGCAGTACCCTTCTGCATACACCAGCCAGCCCGTTTGCGGTGCCGGAT
GCGCTGCAGACTTTCAGCGACGGTGCTCTGGCCGTAGGTGATACGGGTACCATTGCACAC
CTTGGTACATTCACCGAAGTACTTGCAGAAGTGCGTGCCGCATGCCCAGATGCGGAAGT
ACACGATCTGCGTGGTGGCGTTCTGCTGCCTGGCTTTATCGATACTCACGTTCACTATCCG
CAGGTACGCGTTTTGGGAGGGCTGGGGATGGCCTTATTAGAGTGGCTTGATCGTAATACA
CTGCCCGAAGAAGCTCGCTTAGCTGATGCGGCTTATGCTCGCACAATAGCCGGCGAGTTC
CTTCACGGTCTTGCAAGTCATGGAACTACTACCGCCCTGGTGTTTGGTAGTCACTTTGCG
GGAGCTATGGATGAGTTCTTTGCGGAAGCTGCGGCAAGAGGTTTACGGGTGGTTGCGGG
CCAAGTGGTCAGTGATCGTCTGCTGCGCCCCGAATTACACACTACTCCGGAGCGGGCTTA
TGCCGAGGGCAAAGCGCTGATTGAACGTTGGCATGGACAAGGAAGATCTCTGTATGCCG
TGACTCCGCGCTTTTCGCTCTCGGCCAGTGAGGGCATCCTCGATGCGTGTGCGGCATTGC
TGACTGAATTTCCAGACGTGCGGTTTACGAGTCACATAAACGAGAATAATCAGGAGATC
GAAGTGGTGCGGGGGCTGTTCCCAGGTGCACGTGACTACCTCGATACTTATGAAAGAGC
TGGCTTGGTGACGCCGCGCTCTGTTTTTGCCCATAACGTGCACCCCAACGAGCGGGAACT
CGGTGTCTTGGCGGCACAACGGTGCTCTGTCGCGCATTGCCCATGTAGTAATTCAGCCTT
AGGGTCTGGACTTTTCCCACTGCGTCGGCACCTGGCAGCAGGCGTGCATGTAGCGCTGGG
GACGGACGTGGGTGGAGGAACGGGATTCAGCTTACTCAAAGAAGGTTTGCAAGCGTATT
TCATGCAGCAACTGTTGGGTGAAGAAGGTGCGGCACTGTCTCCTGCCCATCTCTTATACT
TAGCAACACTGGCGGGCGCGCAGGCACTTGGTCTGGATGGACAAGTTGGAGATTTCACG
CCTGGAAAACAGTTCGACGCTGTCTGGTTGCGTCCAAGAGCAGGGTCAACCCTGGCAAC
CGTCCTCGCTCATGCTGAGTCTGAAGAACGTACGCTCGCCGCGTTGTTCGCACTCGGTAC
AGGGGATGATGTCGAACGTGTGTGGGTTGGCGGAGGCGTGGTCTTTGCCCGTGAAGCAG
AAGCGTAA
Polypeptide sequence encoded by the polynucleotide of SEQ ID NO: 17:
(SEQ ID NO: 18)
MKIYRSTLLHTPASPFAVPDALQTFSDGALAVGDTGTIAHLGTFTEVLAEVRAACPDAEVHD
LRGGVLLPGFIDTHVHYPQVRVLGGLGMALLEWLDRNTLPEEARLADAAYARTIAGEFLHG
LASHGTTTALVFGSHFAGAMDEFFAEAAARGLRVVAGQVVSDRLLRPELHTTPERAYAEGK
ALIERWHGQGRSLYAVTPRFSLSASEGILDACAALLTEFPDVRFTSHINENNQEIEVVRGLFPG
ARDYLDTYERAGLVTPRSVFAHNVHPNERELGVLAAQRCSVAHCPCSNSALGSGLFPLRRHL
AAGVHVALGTDVGGGTGFSLLKEGLQAYFMQQLLGEEGAALSPAHLLYLATLAGAQALGL
DGQVGDFTPGKQFDAVWLRPRAGSTLATVLAHAESEERTLAALFALGTGDDVERVWVGGG
VVFAREAEA
GDA_10:
(SEQ ID NO: 19)
ATGATGTCAGGAGAACACACGTTAAAAGCGGTACGAGGCAGTTTTATTGATGTCACCCG
TACGATCGATAACCCGGAAGAGATTGCCTCTGCGCTGCGGTTTATTGAGGATGGTTTATT
ACTCATTAAACAGGGAAAAGTGGAATGGTTTGGCGAATGGGAAAACGGAAAGCATCAA
ATTCCTGACACCATTCGCGTGCGCGACTATCGCGGCAAACTGATAGTACCGGGCTTTGTC
GATACACATATCCATTATCCGCAAAGTGAAATGGTGGGGGCCTATGGTGAGCAATTGCT
GGAGTGGTTGAATAAACACACCTTCCCTACTGAACGTCGTTATGAGGATTTAGAGTACGC
CCGCGAAATGTCGGCGTTCTTCATCAAGCAGCTTTTACGTAACGGAACCACCACGGCGCT
GGTGTTTGGCACTGTTCATCCGCAATCTGTTGATGCGCTGTTTGAAGCCGCCAGTCATATC
AATATGCGTATGATTGCCGGTAAGGTGATGATGGACCGCAACGCACCGGATTATCTGCTC
GACACTGCCGAAAGCAGCTATCACCAAAGCAAAGAACTGATCGAACGCTGGCACAAAA
ATGGTCGTCTGCTATATGCGATTACGCCACGCTTCGCCCCGACCTCATCTCCTGAACAGA
TGGCGATGGCGCAACGCCTGAAAGAAGAATATCCGGATACGTGGGTACATACCCATCTC
TGTGAAAACAAAGATGAAATTGCCTGGGTGAAATCGCTTTATCCTGACCATGATGGTTAT
CTGGATGTTTACCATCAGTACGGCCTGACCGGTAAAAACTGTGTCTTTGCTCACTGCGTC
CATCTCGAAGAAAAAGAGTGGGATCGTCTCAGCGAAACCAAATCCAGCATTGCTTTCTGT
CCGACCTCCAACCTTTACCTCGGCAGCGGCTTATTCAACTTGAAAAAAGCATGGCAGAAG
AAAGTTAAAGTGGGCATGGGAACGGATATCGGTGCCGGAACCACTTTCAACATGCTGCA
AACGCTGAACGAAGCCTACAAAGTATTGCAATTACAAGGCTATCGCCTCTCGGCATATG
AAGCGTTTTACCTGGCCACGCTCGGCGGAGCGAAATCTCTGGGCCTTGACGATTTGATTG
GCAACTTTTTACCTGGCAAAGAGGCTGATTTCGTGGTGATGGAACCCACCGCCACTCCGC
TACAGCAGCTGCGCTATGACAACTCTGTTTCTTTAGTCGACAAATTGTTCGTGATGATGA
CGTTGGGCGATGACCGTTCGATCTACCGCACCTACGTTGATGGTCGTCTGGTGTACGAAC
GCAACTAA
Polypeptide sequence encoded by the polynucleotide of SEQ ID NO: 19:
(SEQ ID NO: 20)
MMSGEHTLKAVRGSFIDVTRTIDNPEEIASALRFIEDGLLLIKQGKVEWFGEWENGKHQIPDTI
RVRDYRGKLIVPGFVDTHIHYPQSEMVGAYGEQLLEWLNKHTFPTERRYEDLEYAREMSAF
FIKQLLRNGTTTALVFGTVHPQSVDALFEAASHINMRMIAGKVMMDRNAPDYLLDTAESSY
HQSKELIERWHKNGRLLYAITPRFAPTSSPEQMAMAQRLKEEYPDTWVHTHLCENKDEIAW
VKSLYPDHDGYLDVYHQYGLTGKNCVFAHCVHLEEKEWDRLSETKSSIAFCPTSNLYLGSG
LFNLKKAWQKKVKVGMGTDIGAGTTFNMLQTLNEAYKVLQLQGYRLSAYEAFYLATLGGA
KSLGLDDLIGNFLPGKEADFVVMEPTATPLQQLRYDNSVSLVDKLFVMMTLGDDRSIYRTYV
DGRLVYERN
GDA_11:
(SEQ ID NO: 21)
ATGATGTCAGGAGAACACACGTTAAAAGCGGTACGAGGCAGTTTTATTGATGTCACCCG
TACGGTCGATAACCCGGAAGAGATTGCCTCTGCGCTGCGGTTTATTGAGGATGGTTTATT
ACTCATTAAACAGGGAAAAGTGGAATGGTTTGGCGAATGGGAAAACGGAAAGCATCAA
ATTCCTGACACCATTCGCGTGCGCGACTATCGCGGCAAACTGATAGTACCGGGCTTTGTC
GATACACATATCCATTATCCGCAAAGTGAAATGGTGGGGGCCTATGGTGAGCAATTGCT
GGAGTGGTTGAATAAACACACCTTCCCTACTGAACGGCGTTATGAGGATTTAGAGTACGC
CCGCGAAATGTCGGCGTTCTTCATCAAGCAGCTTTTACGTAACGGAACCACCACGGCGCT
GGTGTTTGGCACTGTTCATCCGCAATCCGTTGATGCGCTGTTTGAAGCCGCCAGTCATAT
CAATATGCGTATGATTGCCGGTAAGGTGATGATGGACCGTAACGCACCGGATTATCTGCT
CGACACTGCCGAAAGCAGCTATCACCAAAGCAAAGAACTGATTGAACGCTGGCACAAAA
ATGGTCGTCTGCTATATGCGATTACGCCACGCTTCGCCCCGACCTCATCTCCTGAACAGA
TGGCGATGGCGCAACGCCTGAAAGAAGAATATCCGGATACGTGGGTACATACCCATCTC
AGTGAAAACAAAGATGAAATTGCCTGGGTGAAATCGCTTTATCCTGACCATGATGGTTAT
CTGGATGTTTACCATCAGTACGGCCTGACCGGTAAAAACTGTGTCTTTGCTCACTGCGTC
CATCTCGAAGAAAAAGAGTGGGATCGTCTCAGCGAAACCAAATCCAGCATTGCTTTCTGT
CCGACCTCCAACCTTTACCTCGGCAGCGGCTTATTCAACTTGAAAAAAGCATGGCAGAAG
AAAGTTAAAGTGGGCATGGGAACGGATATCGGTGCCGGAACCACTTTCAACATGCTGCA
AACGCTGAACGAAGCCTACAAAGTATTGCAATTACAAGGCTATCGCCTCTCGGCATATG
AAGCGTTTTACTTGGCCACGCTCGGCGGAGCGAAATCTCTGGGCCTTGACGATTTGATTG
GCAACTTTTTACCTGGCAAAGAGGCTGATTTCGTGGTGATGGAACCCACCGCCACTCCGC
TACAGCAGCTGCGCTATGACAACTCTGTTTCTTTAGTCGACAAATTGTTCGTGATGATGA
CGTTGGGCGATGACCGTTCGATCTACCGCACCTACGTTGATGGTCGTCTGGTGTACGAAC
GCAACTAA
Polypeptide sequence encoded by the polynucleotide of SEQ ID NO: 21:
(SEQ ID NO: 22)
MMSGEHTLKAVRGSFIDVTRTVDNPEEIASALRFIEDGLLLIKQGKVEWFGEWENGKHQIPD
TIRVRDYRGKLIVPGFVDTHIHYPQSEMVGAYGEQLLEWLNKHTFPTERRYEDLEYAREMSA
FFIKQLLRNGTTTALVFGTVHPQSVDALFEAASHINMRMIAGKVMMDRNAPDYLLDTAESSY
HQSKELIERWHKNGRLLYAITPRFAPTSSPEQMAMAQRLKEEYPDTWVHTHLSENKDEIAW
VKSLYPDHDGYLDVYHQYGLTGKNCVFAHCVHLEEKEWDRLSETKSSIAFCPTSNLYLGSG
LFNLKKAWQKKVKVGMGTDIGAGTTFNMLQTLNEAYKVLQLQGYRLSAYEAFYLATLGGA
KSLGLDDLIGNFLPGKEADFVVMEPTATPLQQLRYDNSVSLVDKLFVMMTLGDDRSIYRTYV
DGRLVYERN
MXMT_10:
(SEQ ID NO: 23)
ATGGAGCTTCAAGAGGTACTGCACATGAACGAGGGCGAAGGAGATACGAGCTACGCGA
AAAATGCGAGTTATAACCTGGCGCTCGCAAAAGTGAAACCTTTCCTGGAACAGTGTATA
CGCGAGTTGCTTCGCGCAAACCTGCCAAACATAAATAAATGCATTAAGGTAGCGGATTT
AGGCTGTGCCTCAGGCCCAAATACGCTGCTCACTGTTCGTGACATAGTACAGTCGATAGA
CAAAGTTGGCCAGGAGGAGAAAAACGAATTAGAACGGCCAACGATACAAATTTTTCTGA
ACGACCTGTTCCAAAACGATTTTAATTCCGTCTTTAAGCTGCTTCCGTCTTTCTATCGGAA
ACTCGAGAAGGAAAATGGTCGTAAAATCGGTAGTTGTCTGATTAGTGCGATGCCCGGCT
CGTTCTATGGTAGACTTTTTCCGGAAGAGTCCATGCATTTTCTGCATTCTTGTTACAGTGT
GCACTGGCTGAGTCAGGTTCCTTCGGGGTTAGTGATCGAACTTGGTATCGGCGCGAACAA
AGGAAGTATCTACAGCTCCAAAGGATGCCGTCCACCGGTCCAGAAGGCCTATTTGGACC
AGTTTACAAAGGACTTCACCACCTTCCTGCGCATACATAGTAAAGAACTGTTTTCTCGTG
GCCGCATGCTCTTAACTTGTATTTGCAAGGTTGATGAGTTTGATGAACCTAACCCTCTGG
ACCTTCTTGACATGGCAATCAATGACCTTATTGTCGAAGGCTTGCTTGAGGAGGAGAAAT
TAGACTCATTCAATATTCCGTTTTTCACGCCATCCGCCGAAGAAGTAAAGTGTATCGTAG
AAGAAGAAGGGAGCTGTGAGATCCTCTATCTCGAGACATTCAAGGCCCACTATGATGCA
GCGTTCTCCATAGACGACGATTACCCTGTTCGGTCACACGAACAAATCAAAGCCGAATAT
GTGGCCAGTCTGATCAGATCTGTCTATGAACCTATCCTCGCCTCCCACTTTGGCGAAGCA
ATAATGCCCGACCTGTTTCACCGCCTTGCTAAGCACGCTGCCAAAGTTTTGCACATGGGG
AAGGGGTGTTACAACAACTTGATCATTTCACTTGCCAAGAAACCGGAGAAGTCTGATGT
GTAA
Polypeptide sequence encoded by the polynucleotide of SEQ ID NO: 23:
(SEQ ID NO: 24)
MELQEVLHMNEGEGDTSYAKNASYNLALAKVKPFLEQCIRELLRANLPNINKCIKVADLGC
ASGPNTLLTVRDIVQSIDKVGQEEKNELERPTIQIFLNDLFQNDFNSVFKLLPSFYRKLEKENG
RKIGSCLISAMPGSFYGRLFPEESMHFLHSCYSVHWLSQVPSGLVIELGIGANKGSIYSSKGCR
PPVQKAYLDQFTKDFTTFLRIHSKELFSRGRMLLTCICKVDEFDEPNPLDLLDMAINDLIVEGL
LEEEKLDSFNIPFFTPSAEEVKCIVEEEGSCEILYLETFKAHYDAAFSIDDDYPVRSHEQIKAEY
VASLIRSVYEPILASHFGEAIMPDLFHRLAKHAAKVLHMGKGCYNNLIISLAKKPEKSDV
MXMT_11:
(SEQ ID NO: 25)
ATGGAACTGCAGGCCGTTTTACACATGAATGGAGGGGAAGGAGACACGAGCTATGCTAA
AAATTCTTCATATAACCTGGCGTTGGCGAAAGTAAAACCAGTGTTAGAACAATGCATTCG
TGAACTGTTACGGGCGAATCTTCCAAATATTAACAATTGTATAAAAGTAGCGGACCTTGG
CTGCGCAAGTGGACCGAACACGTTATTAACTGTTAGAGATATCGTACAAAGTATTGACA
AGGTTGGCCAGGAGGAGAAAAATGAACTGGAGCGGCCGACTATACAGATATTCCTGAAT
GATTTATTTCAGAACGATTTCAATAGCGTTTTCAAGCTGCTTCCTTCGTTTTACCGTAAAC
TTGAGAAAGAAAATGGACGGAAAATTGGGTCATGTTTAATTTCCGCAATGCCCGGCAGT
TTCTATGGGCGCTTGTTTCCGGAAGAAAGCATGCATTTTATTCACTCATGCTATTCGTTCC
ACTGGCTGTCACAAGTGCCAAGTGGACTCGTTATTGAACTGGGGATCTCAGCCAACAAA
GGCAGTATATATTCCTCAAAGGCCTCAAGACCGCCTGTGCAAAAAGCTTACTTAGACCAG
TTCACAAAGGATTTTACAACGTTCCTTCGCATTCACAGCAAGGAATTGTTCTCACGCGGA
CGGATGCTTCTTACATGTATCTGCAAAGTGGATGAGTATGACGAACCAAACCCTCTTGAC
TTGTTAGACATGGCGATAAACGATCTCATTGTGGAAGGTCATCTCGAGGAAGAAAAGTT
AGCTTCTTTTAACCTGCCATTTTTCACACCATCGGCCGAGGAGGTTAAGTGCATCGTTGA
GGAAGAGGGTTCTTTCGAGATCCTCTATCTCGAGACATTTAAAGCGCATTATGATGCGGG
GTTTAGCATAGATGATGATTACCCAGTCCGGTCCCATTTTCAAGTATATGGCGATGAACA
TATTAAAGCAGAGTATGTCGCATCTCTGATCCGCTCCGTTTATGAGCCGATTCTTGCAAG
TCATTTTGGTGAGGCTATCATGCCAGACTTGTTTCACCGGTTAGCCAAACACGCTGCTAA
GGTACTCCATCTGGGTAAGGGTTGTTATAACAATCTCATAATTTCTCTTGCGAAGAAGCC
AGAAAAATCAGACATGTAA
Polypeptide sequence encoded by the polynucleotide of SEQ ID NO: 25:
(SEQ ID NO: 26)
MELQAVLHMNGGEGDTSYAKNSSYNLALAKVKPVLEQCIRELLRANLPNINNCIKVADLGC
ASGPNTLLTVRDIVQSIDKVGQEEKNELERPTIQIFLNDLFQNDFNSVFKLLPSFYRKLEKENG
RKIGSCLISAMPGSFYGRLFPEESMHFIHSCYSFHWLSQVPSGLVIELGISANKGSIYSSKASRP
PVQKAYLDQFTKDFTTFLRIHSKELFSRGRMLLTCICKVDEYDEPNPLDLLDMAINDLIVEGH
LEEEKLASFNLPFFTPSAEEVKCIVEEEGSFEILYLETFKAHYDAGFSIDDDYPVRSHFQVYGD
EHIKAEYVASLIRSVYEPILASHFGEAIMPDLFHRLAKHAAKVLHLGKGCYNNLIISLAKKPEK
SDM
XMT_12:
(SEQ ID NO: 27)
ATGGAATTGCAAGAAGTTCTGCGTATGAACGGTGGCGAAGGTGACACTAGCTACGCTAA
AAATAGTGCTTATAACCAACTGGTACTCGCAAAAGTTAAACCGGTTTTGGAGCAGTGTGT
TCGTGAACTGCTGAGAGCAAACTTACCGAACATAAATAAATGCATCAAAGTTGCCGATTT
GGGCTGTGCAAGCGGCCCTAATACCCTTTTAACCGTTCGGGATATCGTGCAGTCAATCGA
TAAAGTAGGTCAAGAGAAAAAGAATGAATTAGAGCGCCCAACTATCCAGATCTTTCTTA
ACGACCTGTTTCCCAACGACTTCAATAGCGTTTTTAAACTCTTGCCTTCTTTTTATCGGAA
ATTAGAGAAGGAAAATGGGCGTAAGATCGGTTCCTGCCTTATCGGTGCGATGCCAGGTT
CATTTTACTCCCGTTTATTTCCGGAAGAAAGTATGCATTTTCTCCACTCTTGCTATTGTCTT
CAGTGGTTATCTCAGGTCCCTAGTGGATTGGTAACAGAATTAGGGATTAGCACTAACAAG
GGCTCCATTTATAGTTCGAAAGCCTCACGTCTGCCTGTACAAAAGGCATATCTTGATCAG
TTTACGAAAGACTTTACTACCTTCCTGAGAATCCATTCAGAGGAGTTGTTTTCGCATGGG
CGGATGCTTCTTACATGTATATGCAAAGGAGTTGAATTAGACGCAAGAAATGCTATCGAT
CTGTTAGAGATGGCAATTAACGACTTAGTAGTCGAGGGTCATCTTGAAGAGGAAAAACT
TGACTCTTTTAACCTGCCGGTCTATATTCCTTCAGCCGAGGAAGTTAAATGCATTGTAGA
AGAAGAGGGGTCCTTTGAGATACTCTATCTTGAGACTTTTAAGGTCTTATACGACGCGGG
GTTCTCAATCGACGATGAGCACATCAAAGCAGAATATGTTGCCTCTTCGGTGCGGGCGGT
ATATGAACCGATATTAGCCAGTCATTTCGGAGAAGCGATTATACCGGATATCTTTCATCG
TTTTGCTAAGCATGCCGCTAAAGTACTCCCTTTGGGGAAAGGATTTTATAACAATCTGAT
CATTTCACTTGCCAAGAAACCCGAGAAATCTGATGTGTAA
Polypeptide sequence encoded by the polynucleotide of SEQ ID NO: 27:
(SEQ ID NO: 28)
MELQEVLRMNGGEGDTSYAKNSAYNQLVLAKVKPVLEQCVRELLRANLPNINKCIKVADLG
CASGPNTLLTVRDIVQSIDKVGQEKKNELERPTIQIFLNDLFPNDFNSVFKLLPSFYRKLEKEN
GRKIGSCLIGAMPGSFYSRLFPEESMHFLHSCYCLQWLSQVPSGLVTELGISTNKGSIYSSKAS
RLPVQKAYLDQFTKDFTTFLRIHSEELFSHGRMLLTCICKGVELDARNAIDLLEMAINDLVVE
GHLEEEKLDSFNLPVYIPSAEEVKCIVEEEGSFEILYLETFKVLYDAGFSIDDEHIKAEYVASSV
RAVYEPILASHFGEAIIPDIFHRFAKHAAKVLPLGKGFYNNLIISLAKKPEKSDV
XMT_13:
(SEQ ID NO: 29)
ATGGAATTGCAAGAGGTGTTGCGCATGAACGGAGGGGAAGGGGATACTAGTTATGCAAA
GAATTCCGCCTATAATCAGTTGGTACTGGCCAAGGTCAAACCCGTATTAGAGCAATGCGT
GCGGGAGTTATTACGTGCGAATCTGCCAAACATCAATAAATGTATAAAAGTTGCAGACTT
GGGTTGTGCCAGTGGGCCTAACACACTTCTGACGGTAAGAGACATCGTTCAGTCCATCGA
TAAAGTCGGACAAGAGAAGAAAAACGAATTAGAGCGCCCGACGATCCAAATTTTCCTTA
ATGATCTGTTCCCCAACGACTTCAACTCGGTCTTCAAACTTCTGCCCTCGTTTTATCGCAA
GTTGGAAAAGGAGAATGGTCGCAAAATTGGTTCGTGTTTAATCGGCGCGATGCCGGGTA
GTTTTTATTCGCGCTTGTTTCCAGAAGAATCAATGCATTTCTTACACTCGTGTTACTGCTT
ACAGTGGTTGTCACAGGTCCCTTCAGGATTGGTAACTGAACTGGGGATTAGCACAAACA
AAGGGTCAATATATTCAAGTAAAGCTAGTAGACTGCCCGTGCAAAAGGCATATCTGGAT
CAGTTCACAAAAGATTTTACTACCTTTCTGCGTATCCATAGTGAAGAGCTGTTCTCGCAT
GGTCGGATGTTATTAACCTGTATATGTAAGGGTGTTGAACTCGATGCACGGAATGCCATC
GACCTGCTTGAAATGGCGATTAATGATCTGGTGGTGGAGGGACACCTCGAAGAAGAAAA
GCTGGACTCGTTCAACCTCCCAGTTTATATACCGAGTGCTGAAGAAGTTAAGTGCATCGT
GGAAGAAGAGGGTAGCTTTGAAATCCTGTACCTTGAAACGTTCAAAGTGCTGTACGACG
CAGGATTTTCAATTGATGATGACTATCCCGTGCGCTCACATTTTCAAGTATATGGAGATG
AACACATCAAAGCAGAATATGTAGCGAGTTTAATCCGTAGTGTCTATGAGCCAATCTTAG
CAAGTCATTTCGGTGAGGCGATTATGCCAGACTTGTTTCATCGCCTGGCTAAACACGCCG
CTAAAGTTCTCCACCTTGGAAAAGGGTTCTATAATAATCTGATAATCTCTCTTGCCAAAA
AGCCCGAAAAATCCGATGTCTAA
Polypeptide sequence encoded by the polynucleotide of SEQ ID NO: 29:
(SEQ ID NO: 30)
MELQEVLRMNGGEGDTSYAKNSAYNQLVLAKVKPVLEQCVRELLRANLPNINKCIKVADLG
CASGPNTLLTVRDIVQSIDKVGQEKKNELERPTIQIFLNDLFPNDFNSVFKLLPSFYRKLEKEN
GRKIGSCLIGAMPGSFYSRLFPEESMHFLHSCYCLQWLSQVPSGLVTELGISTNKGSIYSSKAS
RLPVQKAYLDQFTKDFTTFLRIHSEELFSHGRMLLTCICKGVELDARNAIDLLEMAINDLVVE
GHLEEEKLDSFNLPVYIPSAEEVKCIVEEEGSFEILYLETFKVLYDAGFSIDDDYPVRSHFQVY
GDEHIKAEYVASLIRSVYEPILASHFGEAIMPDLFHRLAKHAAKVLHLGKGFYNNLIISLAKKP
EKSDV
MXMT_14:
(SEQ ID NO: 31)
ATGGAACTCCAGGAAGTTCTTCGGATGAATGGTGGCGAAGGCGACACAAGCTATGCCAA
AAATTCGTCATATAATCTTGCCCTGGCGAAAGTAAAACCCGTTTTGGAACAGTGTATCCG
GGAATTATTACGTGCTAACCTGCCCAACATTAATAATTGCATTAAAGTAGCTGATCTCGG
TTGCGCATCGGGACCGAATACGTTACTTACTGTCAGAGACATCGTTCAGTCGATTGACAA
ACTTGGGCTGGAGGAAAAGAATGAGCTGGAACGCCCGACAATACAGATATTTCTGAACG
ATCTGTTTCAGAACGACTTTAACTCTGTCTTTAAGCTGTTACCCAGTTTCTACCGCAAATT
GGAAAAAGAAAACGGCCGTAAGATTGGCTCCTGCCTCATTTCCGCTATGCCGGGTTCATT
CTATGGTAGATTGTTCCCCGAGGAATCAATGCACTTCCTGCATTCATGTTACTGCCTGCAC
TGGTTGTCCCAGGTTCCCTCGGGCCTTGTCACGGAACTGGGTATCTCAGCAAATAAGGGA
AGTATATACTCTTCAAAGGCCAGTCGCCTGCCGGTTAGAAAAGCGTATCTTGACCAATTC
ACTAAGGATTTCACCACTTTCTTGCGCATCCACTCAGAGGAGCTGTTTTCTCATGGAAGA
ATGCTGCTCACTTGCATTTGCAAAGGGGTAGAATTTGATGCCCGGAATGCCATAGATTTA
TTAGAAATGGCTATTAATGACCTGGTGGTGGAGGGTCACTTGGAAGAGGAAAAATTGGA
TAGCTTTAACCTGCCGGTGTATATTCCGTCAACAGAGGAGGTGAAGTGCATCGTAGAAG
AAGAAGGCAGTTTCGAGATTCTGTACCTGGAAACTTTCAAAGTTCTGTACGATGCAGGTT
TCTCAATTGACGATGATTATCCGCTCCGTAGTCATGTCCAGGTGTATTCAGATGAGCATA
TTAAGGCGGAATACGTTGCGTCATCGGTACGGGCAGTATATGAGCCTATCCTTGCATCAC
ACTTTGGTGAAGCTATCATTCCTGATATCTTTCATCGGTTCGCTAAACATGCGGCGAAAG
TGCTGCCTTTAGGTAAAGGCTTTTATAACAACCTTATCATCTCTCTGGCTAAAAAGCCTG
AAAAGTCCGACGTGAACCACTAA
Polypeptide sequence encoded by the polynucleotide of SEQ ID NO: 31:
(SEQ ID NO: 32)
MELQEVLRMNGGEGDTSYAKNSSYNLALAKVKPVLEQCIRELLRANLPNINNCIKVADLGC
ASGPNTLLTVRDIVQSIDKLGLEEKNELERPTIQIFLNDLFQNDFNSVFKLLPSFYRKLEKENG
RKIGSCLISAMPGSFYGRLFPEESMHFLHSCYCLHWLSQVPSGLVTELGISANKGSIYSSKASR
LPVRKAYLDQFTKDFTTFLRIHSEELFSHGRMLLTCICKGVEFDARNAIDLLEMAINDLVVEG
HLEEEKLDSFNLPVYIPSTEEVKCIVEEEGSFEILYLETFKVLYDAGFSIDDDYPLRSHVQVYSD
EHIKAEYVASSVRAVYEPILASHFGEAIIPDIFHRFAKHAAKVLPLGKGFYNNLIISLAKKPEKS
DVNH
DXMT_15:
(SEQ ID NO: 33)
ATGGAACTTCAGGAAGTCCTTCACATGAATGAGGGAGAGGGAGATACCTCTTACGCCAA
GAACGCTTCTTATAACCTGGCGTTGGCCAAAGTGAAGCCTTTCCTTGAACAGTGCATTAG
AGAATTACTGCGGGCCAATTTACCAAACATTAATAAGTACATCAAAGTGGCTGACTTAG
GTTGCGCGAGCGGTCCAAACACGCTCCTGACCGTGCGTGATATCGTACAATCAATAGATA
AAGTTGGACAGGAGGAGAAGAATGAACTGGAGCGGCCGACGATACAAATCTTTCTGAAT
GATCTCTTTCAAAACGATTTCAACTCGGTGTTTAAGCTGCTGCCAAGTTTCTATCGGAAA
CTTGAAAAGGAAAACGGGCGTAAAATTGGAAGTTGCCTGATTTCAGCCATGCCAGGTTC
TTTTTATGGCCGTTTATTCCCCGAGGAATCAATGCACTTTCTTCACAGTTGCTACTCAGTC
CATTGGCTGTCGCAGGTTCCTTCCGGCCTTGTAATCGAACTGGGTATTGGTGCAAATAAA
GGGAGCATCTACTCTTCAAAAGGGTGCCGTCCGCCTGTGCAAAAAGCTTATTTGGATCAA
TTTACGAAAGACTTCACTACGTTTTTGCGGATACATTCCGAAGAGCTGTTTTCTCACGGA
CGCATGCTCCTGACATGCATCTGCAAAGGTGTTGAGTTGGATGCCCGCAATGCCATCGAT
TTGCTTGAAATGGCAATTAATGACCTCGTAGTGGAAGGCCATCTGGAAGAGGAGAAGCT
TGACTCTTTCAACTTGCCTGTATACATACCATCTGCCGAAGAAGTAAAATGCATCGTGGA
GGAAGAAGGTTCTTTCGAGATCTTGTATCTTGAAACATTTAAGGTCTTGTATGATGCTGG
CTTTAGTATAGATGATGAGCACATCAAGGCGGAGTATGTCGCCTCTTCAGTTAGAGCCGT
TTATGAACCTATATTGGCGAGCCATTTCGGAGAGGCCATTATACCCGATATCTTCCATCG
GTTTGCCAAACATGCCGCAAAAGTTTTACCCCTTGGCAAAGGTTTCTACAACAACCTGAT
TATTTCGCTCGCGAAGAAACCCGAAAAATCTGATGTTTAA
Polypeptide sequence encoded by the polynucleotide of SEQ ID NO: 33:
(SEQ ID NO: 34)
MELQEVLHMNEGEGDTSYAKNASYNLALAKVKPFLEQCIRELLRANLPNINKYIKVADLGC
ASGPNTLLTVRDIVQSIDKVGQEEKNELERPTIQIFLNDLFQNDFNSVFKLLPSFYRKLEKENG
RKIGSCLISAMPGSFYGRLFPEESMHFLHSCYSVHWLSQVPSGLVIELGIGANKGSIYSSKGCR
PPVQKAYLDQFTKDFTTFLRIHSEELFSHGRMLLTCICKGVELDARNAIDLLEMAINDLVVEG
HLEEEKLDSFNLPVYIPSAEEVKCIVEEEGSFEILYLETFKVLYDAGFSIDDEHIKAEYVASSVR
AVYEPILASHFGEAIIPDIFHRFAKHAAKVLPLGKGFYNNLIISLAKKPEKSDV
DXMT_16:
(SEQ ID NO: 35)
ATGGAACTCCAGGAGGTGCTGCAGATGAATGGCGGTGAAGGGGATACATCCTATGCCAA
GAATTCGGCTTATAATCAGTTGGTGCTTGCTAAGGTGAAACCTGTCCTTGAGCAGTGCGT
CAGAGAACTCCTTCGCGCTAACCTTCCGAACATTAATAAATGCATTAAAGTCGCTGACTT
GGGCTGCGCGAGTGGACCCAATACCTTATTAACTGTTCGCGACATAGTCCAGTCCATAGA
CAAAGTGGGCCAGGAGAAGAAGAACGAACTGGAACGCCCGACTATTCAAATTTTTCTGA
ATGACCTTTTCCCGAACGATTTCAATAGCGTGTTTAAACTCTTGCCTTCGTTTTACAGAAA
ACTGGAAAAGGAAAATGGGCGTAAAATTGGGTCGTGCTTGATCGGAGCCATGCCTGGCT
CTTTTTATTCACGGCTGTTTCCGGAAGAATCTATGCATTTCTTACACAGCTGTTATTCAGT
GCATTGGCTGTCACAGGTCCCGAGTGGATTAGTGACAGAACTGGGCATCTCTGCAAACA
AGGGGTGTATCTATTCATCTAAAGCAAGCCGGCCCCCAGTACAGAAGACGTACCTTGAC
CAATTCACCAAAGACTTTACGACTTTCCTGCGTATTCACTCGGAAGAGCTGATTTCGCGC
GGAAGAATGTTGTTGACTTTTATCTGTAAGGAAGACGAATTCGGAAACCCTAATAGTATG
GACCTGTTAGAGATGTCAATAAATGACTTGGTGATCGAGGGTCATCTCGAAGAGGAAAA
GCTTGATTCCTTTAATGTCCCAGTTTATGCCGCCAGTGCAGAAGAAGTGAAGCGTATAGT
AGAGGAGGAAGGTTCCTTCGAGATTCTGTATTTGGAGACGTTTAAGGCCCCGTACGACGC
TGGCTTCTCTATCGATGACGACTATCAAGGTCGGAGTCACAGTCCGGTGTCATGTGATGA
GCATGCGAGAGCGGCTCATGTTGCGAGTGTTGTACGGAGCGTGTACGAGCCTATCCTGGC
AGGACACTTTGGTGAGGCAATCTTACCGGACCTCTCTCACAGAATCGAGAAAAATGCTG
CGAAAGTATTACGTTCTGGCAAAGGATTCTATGACTCGCTGATCATAAGTTTGGCTAAGA
AGCCGGAAAAATCTGACGTGTAA
Polypeptide sequence encoded by the polynucleotide of SEQ ID NO: 35:
(SEQ ID NO: 36)
MELQEVLQMNGGEGDTSYAKNSAYNQLVLAKVKPVLEQCVRELLRANLPNINKCIKVADL
GCASGPNTLLTVRDIVQSIDKVGQEKKNELERPTIQIFLNDLFPNDFNSVFKLLPSFYRKLEKE
NGRKIGSCLIGAMPGSFYSRLFPEESMHFLHSCYSVHWLSQVPSGLVTELGISANKGCIYSSKA
SRPPVQKTYLDQFTKDFTTFLRIHSEELISRGRMLLTFICKEDEFGNPNSMDLLEMSINDLVIEG
HLEEEKLDSFNVPVYAASAEEVKRIVEEEGSFEILYLETFKAPYDAGFSIDDDYQGRSHSPVSC
DEHARAAHVASVVRSVYEPILAGHFGEAILPDLSHRIEKNAAKVLRSGKGFYDSLIISLAKKPE
KSDV
DXMT_17:
(SEQ ID NO: 37)
ATGGAACTGCAGGAAGTGTTGCACATGAACGGCGGCGAGGGCGATACCTCCTATGCTAA
GAATTCAAGTTATAATCTGTTTCTGATTCGGGTTAAACCGGTACTGGAACAGTGCATACA
AGAATTGCTGCGTGCGAATTTACCTAACATAAATAAGTGTTTTAAAGTGGGTGATCTTGG
GTGTGCGTCAGGACCGAACACTTTTTCTACCGTTCGGGACATCGTGCAGTCAATCGATAA
AGTCGGTCAGGAGAAAAAGAACGAGTTGGAACGTCCAACGATACAAATCTTCCTCAATG
ACCTTTTCCAGAACGACTTTAACTCGGTTTTTAAGTTACTGCCCTCCTTCTATCGTAATCT
GGAAAAAGAGAATGGCCGGAAAATTGGTTCATGTCTGATTGGTGCGATGCCGGGCTCAT
TTTATTCACGTTTGTTTCCAGAGGAAAGCATGCATTTTCTCCATTCCTGTTACTGCCTCCA
TTGGTTGAGCCAGGTTCCTTCTGGCCTTGTCACAGAACTCGGTATTTCTGTGAATAAAGG
CTGTATTTATTCCTCGAAGGCCTCCAGACCACCTATCCAGAAAGCGTATTTAGATCAGTT
TACAAAGGATTTTACCACGTTTCTGAGAATTCACTCTGAAGAGCTCATATCACGCGGTAG
AATGCTGTTAACGTTCATTTGTAAAGAAGATGAGTTCGACCACCCAAATTCAATGGATCT
GCTGGAGATGTCTATAAATGATCTGGTTGTCGAGGGTCATTTAGAAGAGGAAAAACTGG
ATTCCTTTAACGTCCCCATTTATGCTCCCAGCACGGAAGAGGTGAAACGCATCGTTGAAG
AAGAAGGGTCATTTGAAATCTTATATTTAGAAACGTTTTATGCACCTTATGATGCGGGCT
TTTCCATCGATGACGACTATCAAGGTCGCTCCCACAGTCCAGTCAGTTGCGATGAGCATG
CTCGGGCTGCACATGTTGCTTCAGTTGTGCGCTCTATTTACGAACCAATTCTTGCATCACA
TTTCGGAGAGGCTATTCTTCCGGACCTTTCTCACCGCATTGCGAAAAACGCAGCAAAAGT
CTTACGCTCGGGTAAGGGGTTTTATGATTCCGTTATAATCAGTCTCGCAAAGAAGCCTGA
AAAAGCTGATATGTAA
Polypeptide sequence encoded by the polynucleotide of SEQ ID NO: 37:
(SEQ ID NO: 38)
MELQEVLHMNGGEGDTSYAKNSSYNLFLIRVKPVLEQCIQELLRANLPNINKCFKVGDLGCA
SGPNTFSTVRDIVQSIDKVGQEKKNELERPTIQIFLNDLFQNDFNSVFKLLPSFYRNLEKENGR
KIGSCLIGAMPGSFYSRLFPEESMHFLHSCYCLHWLSQVPSGLVTELGISVNKGCIYSSKASRP
PIQKAYLDQFTKDFTTFLRIHSEELISRGRMLLTFICKEDEFDHPNSMDLLEMSINDLVVEGHL
EEEKLDSFNVPIYAPSTEEVKRIVEEEGSFEILYLETFYAPYDAGFSIDDDYQGRSHSPVSCDEH
ARAAHVASVVRSIYEPILASHFGEAILPDLSHRIAKNAAKVLRSGKGFYDSVIISLAKKPEKADM
DXMT_18:
(SEQ ID NO: 39)
ATGGAACTGCAGGAGGTCCTGCACATGAATGGTGGTGAGGGTGATACCTCGTACGCCAA
AAATAGTTTTTATAACCTTTTTCTGATTCGTGTCAAACCCATACTCGAGCAGTGTATACAA
GAATTACTGCGCGCTAACTTGCCGAATATCAACAAGTGCATAAAAGTTGCCGACCTGGGT
TGCGCTTCAGGGCCTAATACGTTGTTGACAGTGCGTGATATTGTTCAGTCTATTGATAAG
GTCGGACAAGAGAAGAAAAATGAACTGGAACGCCCTACAATCCAGATATTTCTGAATGA
TCTGTTTCAGAACGACTTTAATAGTGTGTTTAAAAGCCTTCCGTCATTCTACCGCAAGCTG
GAGAAAGAAAATGGGTGTAAGATTGGCAGTTGTTTGATAGGTGCGATGCCAGGAAGCTT
TTACGGCCGGCTGTTTCCCGAGGAAAGCATGCACTTCTTACACAGTTGTTATTGTTTGCAT
TGGCTGTCCCAGGTGCCTAGTGGGTTAGTAACCGAGCTCGGGATCAGTGCCAATAAAGG
CTGTATCTACTCTAGCAAAGCGAGCCGGCCTCCAATCCAAAAAGCGTACCTGGATCAGTT
TACTAAGGACTTTACGACATTCCTGCGTATCCATAGCGAAGAATTAATCTCGCGCGGTCG
CATGCTGCTGACCTGGATCTGTAAGGAAGATGAGTTCGAAAACCCAAATAGTATAGATC
TCCTCGAGATGAGCATTAACGATCTCGTGATAGAGGGTCACTTAGAGGAGGAAAAACTG
GACAGTTTCAATGTCCCAATCTATGCGCCATCCACGGAGGAAGTCAAGTGCATAGTGGA
AGAAGAAGGCTCCTTTGAGATTCTTTATCTTGAGACTTTCAAAGTACCGTATGACGCGGG
ATTTAGCATCGATGATGATTACCAAGGAAGATCTCACTCTCCAGTTTCATGCGATGAACA
CGCAAGAGCGGCGCATGTAGCCAGCGTTGTCCGTTCTATATTTGAGCCAATTGTGGCGTC
CCACTTTGGCGAGGCTATCCTGCCTGATCTGAGCCATCGGATAGCCAAGAATGCGGCTAA
AGTTTTACGTTCTGGTAAAGGGTTCTATGATTCAGTCATCATCTCGCTGGCTAAAAAGCC
TGAGAAGGCAGATATGTAA
Polypeptide sequence encoded by the polynucleotide of SEQ ID NO: 39:
(SEQ ID NO: 40)
MELQEVLHMNGGEGDTSYAKNSFYNLFLIRVKPILEQCIQELLRANLPNINKCIKVADLGCAS
GPNTLLTVRDIVQSIDKVGQEKKNELERPTIQIFLNDLFQNDFNSVFKSLPSFYRKLEKENGCK
IGSCLIGAMPGSFYGRLFPEESMHFLHSCYCLHWLSQVPSGLVTELGISANKGCIYSSKASRPPI
QKAYLDQFTKDFTTFLRIHSEELISRGRMLLTWICKEDEFENPNSIDLLEMSINDLVIEGHLEEE
KLDSFNVPIYAPSTEEVKCIVEEEGSFEILYLETFKVPYDAGFSIDDDYQGRSHSPVSCDEHAR
AAHVASVVRSIFEPIVASHFGEAILPDLSHRIAKNAAKVLRSGKGFYDSVIISLAKKPEKADM
DXMT_19:
(SEQ ID NO: 41)
ATGGAATTGCAAGAGGTTTTACGCATGAATGGCGGTGAAGGCGACACTAGCTATGCTAA
AAATTCGTCATACAACCTGTTTCTGATTCGGGTCAAACCGGTACTTGAACAATGCATTCA
AGGGTTACTCAGAGCCAACCTGCCTAACATTAACAAATGCATTAAAGTAGCCGATTTAG
GATGCGCAAGTGGAAGCAATACATTAAGTACGGTCAGAGATATAGTGCAATCTATAGAC
AAGGTTGGACAAGAAAAGAAGAATGAATTGGAAAGACCAACAATTCAAGTCTTTCTGAA
CGACTTGTTTCAGAACGACTTTAATTCCGTGTTTAAGCTGTTGCCGTCCTTTTATCGGAAA
CTGGAAAAGGAAAATGGCCGGAAAATTGGAAGTTGCTTGATTGGGGCCATGCCTGGTTC
CTTCTATGGTCGGCTGTTCCCAGAGGAAAGTATGCACTTTCTTCACTCCTGTTATTGCCTG
CATTGGCTCTCTCAGGTACCATCAGGCCTGGTTACAGAGTTGGGAATTTCAGCAAATAAG
GGGTGCATTTATTCGTCCAAAGCCAGCAGACCGCCGATCAAAAAGGCCTATCTTGACCA
ATTCACGAAGGACTTCACTACGTTTTTACGGGTTCACAGCGAAGAATTGATAAGTCGTGG
AAGAATGTTGTTGACTTGGATCTGTAAAGAAGACGAGTTTGAAAACCCAAATTCAATTG
ATCTGCTGGAGATGTCGCTTAATGATCTGGTTATTGAGGGCCATCTCGAGGAAGAAAAAC
TGGATTCCTTTAACGTACCGATATTCGCCCCGTCCACGGAAGAGGTCAAATGTATAGTAG
AAGAAGAAGGGTCATTTGAGATACTTTACTTAGAGACATTCAAAACACCATACGACGCA
GGGTTTTCAATCGACGATGATTATCAGGGTAGAAGCCATTCGCCGGTTTCATGTGACGAA
CATGCGATTGCAGAACATGTCGCCGCAGTGGTGAGATCGATTTACGAGCCTATATTAGCT
TCACATTTTGGTGAGGCAATTATGCCGGATCTTAGTCATCGTATAGCCAAAAATGCTGCG
AAGGTCTTGCGCTCAGGTAAGGGTTTTTATGACTCAATTATCATAAGTCTTGCCAAGAAG
CCGGAGAAATCAGATGTTAATCATTAA
Polypeptide sequence encoded by the polynucleotide of SEQ ID NO: 41:
(SEQ ID NO: 42)
MELQEVLRMNGGEGDTSYAKNSSYNLFLIRVKPVLEQCIQGLLRANLPNINKCIKVADLGCA
SGSNTLSTVRDIVQSIDKVGQEKKNELERPTIQVFLNDLFQNDFNSVFKLLPSFYRKLEKENGR
KIGSCLIGAMPGSFYGRLFPEESMHFLHSCYCLHWLSQVPSGLVTELGISANKGCIYSSKASRP
PIKKAYLDQFTKDFTTFLRVHSEELISRGRMLLTWICKEDEFENPNSIDLLEMSLNDLVIEGHL
EEEKLDSFNVPIFAPSTEEVKCIVEEEGSFEILYLETFKTPYDAGFSIDDDYQGRSHSPVSCDEH
AIAEHVAAVVRSIYEPILASHFGEAIMPDLSHRIAKNAAKVLRSGKGFYDSIIISLAKKPEKSDV
NH
DXMT_20:
(SEQ ID NO: 43)
ATGGAAGTGCAGGAAGTCCTTAGAATGAATGGTGGCGAAGGTGATACATCATACGCGAA
AAATTCAAGCTATAACCTCTTCCTTATTCGGGTCAAACCCGTCCTCGAACAGTGCATTCA
AGAACTGTTAAGAGCAAATCTGCCTAACATAAATAAATGTATTAAAGTTGCTGACCTCGG
CTGCGCGTCTGGGTCAAACACTCTTTCAACCGTACGGGGCATAGTCCAGATCATCGATAA
AGTGGGTCAGGAAAAGAAAAATGAACTTGAACGCCCGACAATACAGGTGTTTTTAAACG
ATCTGTTTCAAAACGACTTTAACTCTGTCTTTAAATCACTTCCTTCTTTCTACCGCAAGCT
GGAAAAGGAAAACGGACGCAAAATTGGCTCCTGCTTAATCGGGGCAATGCCGGGGAGCT
TTTATGGTCGTCTGTTCCCCGAAGAAAGCATGCACTTCTTGCATTCATGTTATTGCTTGCA
CTGGCTGTCTCAGGTGCCGAGTGGCTTAGTGACTGAGCTGGGTATTTCTGCGAACAAAGG
GTGTATTTATTCGTCAAAGGCGTCACGGCCACCTATAAAGAAGGCATATCTGGATCAATT
CACGAAGGATTTTACAACCTTCCTGAAAATACACAGTGAGGAGTTGATTTCTCGCGGTCG
CATGCTGCTGACGTGGATCTGCAAGGAAGATGAGTTTGAGAATCCCAATTCAATTGATCT
GCTCGAAATGTCCCTGAATGATTTGGTGACAGAGGGCCTCTTGGAAGAAGAGAAGCTGG
ACTCTTTTAATGTGCCTATCTATGCCCCATCGACAGAAGTGGTTAAATGCATTGTAGAAG
AAGAAGGCTCATTTGAAATACTCTATTTGAAAACATTCAAAGCTCCGTACGATGCAGGAT
TTTCGACCGATGACGATTATCAAGGGCGTTCTCATAGCCCAGCGAGTTGTGACGAACATG
CTCGTGCCGCTCATGTAGCTTCTGTGGCACGCTCTATCTTTGAGCCGATAGTAGCCTCCCA
TTTTGGAGAAGCAATAATGCCAGACTTGTCCCATCGCATAGCTAAAAATGCGGCAAAAG
TTTTACGCAGCGGAAAGGGCTTTTTCGACAGTATTATAATTAGCCTGGCGAAAAAGCCAG
AAAAATCAGATATGTAA
Polypeptide sequence encoded by the polynucleotide of SEQ ID NO: 43:
(SEQ ID NO: 44)
MEVQEVLRMNGGEGDTSYAKNSSYNLFLIRVKPVLEQCIQELLRANLPNINKCIKVADLGCA
SGSNTLSTVRGIVQIIDKVGQEKKNELERPTIQVFLNDLFQNDFNSVFKSLPSFYRKLEKENGR
KIGSCLIGAMPGSFYGRLFPEESMHFLHSCYCLHWLSQVPSGLVTELGISANKGCIYSSKASRP
PIKKAYLDQFTKDFTTFLKIHSEELISRGRMLLTWICKEDEFENPNSIDLLEMSLNDLVTEGLL
EEEKLDSFNVPIYAPSTEVVKCIVEEEGSFEILYLKTFKAPYDAGFSTDDDYQGRSHSPASCDE
HARAAHVASVARSIFEPIVASHFGEAIMPDLSHRIAKNAAKVLRSGKGFFDSIIISLAKKPEKSDM
DXMT_21:
(SEQ ID NO: 45)
CTGCAGGAAGTCCTGCACATGAACGGCGGAGAAGGAGAAGCTAGCTATGCTAAGAATTC
CAGTTTCAATCAACTGGTGCTGGCCAAGGTGAAACCTGTCTTAGAGCAGTGCGTACGTGA
ACTGCTGCGGGCCAATTTACCAAACATCAACAAGTGTATAAAGGTTGCCGATTTGGGCTG
CGCTTCGGGCCCAAATACACTGTTAACGGTATGGGATACAGTACAGTCTATTGATAAGGT
TAAACAGGAAATGAAAAACGAACTGGAGCGCCCCACTATTCAAGTGTTTCTGACGGACC
TTTTCCAGAACGATTTCAATTCCGTGGTGATGTTGTTGCCATCCTTTTACCGGAAACTGGA
GAAGGAGAACGGACGTAAAATCGGATCTTGTCTGATCGCGGCAATGCCGGGCTCTTTTC
ACGGACGCTTATTTCCAGAAGAATCCATGCACTTCTTACATTCCTCTTATTCCATCCAATT
CCTGTCACAAGTTCCGAGCGGGTTGGTTACGGAACTCGGAATCACGGCAAATAACCGTTC
AATTTATTCGAGTAAAGCGTCCCCGCCTCCGGTCCAGAAAGCGTACTTAGACCAGTTCAC
TAAAGACTTCACGACGTTTTTACGGATGAGATCGGAGGAACTGCTCAGTCGTGGCAGAA
TGCTGCTGACCTGCATTTGCAAGGGGGACGAGTGCGATGGTCCAAATACAATGGATTTGT
TGGAAATGGCTATCAACGACTTAGTAGCTGAGGGTCGTCTGGGTGAAGAAAAATTAGAT
TCATTTAACGTGCCCATCTATACAGCGTCCGTGGAAGAAGTCAAATGCATGGTCGAGGA
AGAGGGTAGCTTTGAGATACTGTACCTGCAGACTTTTAAATTACGCTATGATGCAGGTTT
TAGTATCGACGATGATTGTCAAGTACGCTCCCATAGTCCGGTTTATTCTGATGAGCACGC
CAGAGCTGCACATGTAGCATCTCTCATACGCTCAGTTTATGAACCAATCTTAGCATCCCA
TTTTGGTGAAGTGATAAAACCGGATATCTTCCATCGTTTTGCGACCAACGCGGCGAAAGT
AATACGGCTGGGTAAAGGCTTTTACAACAATTTGATAATTAGCCTTGCCAAGAAACCCGA
GAAATCAGATATTTAA
Polypeptide sequence encoded by the polynucleotide of SEQ ID NO: 45:
(SEQ ID NO: 46)
MQEVLHMNGGEGEASYAKNSSFNQLVLAKVKPVLEQCVRELLRANLPNINKCIKVADLGCA
SGPNTLLTVWDTVQSIDKVKQEMKNELERPTIQVFLTDLFQNDFNSVVMLLPSFYRKLEKEN
GRKIGSCLIAAMPGSFHGRLFPEESMHFLHSSYSIQFLSQVPSGLVTELGITANNRSIYSSKASP
PPVQKAYLDQFTKDFTTFLRMRSEELLSRGRMLLTCICKGDECDGPNTMDLLEMAINDLVAE
GRLGEEKLDSFNVPIYTASVEEVKCMVEEEGSFEILYLQTFKLRYDAGFSIDDDCQVRSHSPV
YSDEHARAAHVASLIRSVYEPILASHFGEVIKPDIFHRFATNAAKVIRLGKGFYNNLIISLAKKP
EKSDI
DXMT_22:
(SEQ ID NO: 47)
ATGGAACTCCAGGAGGTTCTCCACATGAATGGTGGCGAAGGAGAGGCAAGTTACGCAAA
GAACTCCTCATTCAATCAATTGGTCTTAGCGAAGGTGAAACCCGTTCTGGAACAATGTGT
GCGTGAACTGCTTCGCGCAAATCTTCCGAATATCAATAAATGCATTAAGGTAGCGGATCT
TGGGTGCGCGTCAGGTCCGAATACCCTCTTAACGGTCTGGGATACCGTTCAATCCATCGA
TAAAGTCAAGCAGGAAATGAAAAATGAGTTGGAACGCCCGACCATACAGGTGTTTCTGA
CCGATCTTTTCCAAAACGATTTTAACTCGGTGTTCATGTTGCTGCCCTCATTTTATCGCAA
ATTAGAGAAGGAAAATGGACGCAAAATTGGCTCTTGTCTTATTGCCGCTATGCCCGGAA
GCTTTCATGGTCGGCTTTTCCCAGAGGAGTCCATGCATTTCTTACATTCCAGCTACTCTTT
GCAGTTTCTCAGCCAAGTCCCCTCTGGGTTGGTCACCGAGTTAGGGATAACAGCTAATAA
ACGCTCAATATACTCTTCGAAAGCAAGTCCGCCTCCAGTCCAGAAAGCATACCTTGACCA
GTTCACCAAAGATTTCACGACATTCCTCCGCATGCGTTCCGAAGAGTTACTCAGTCGGGG
CCGCATGTTGCTTACCTGTATTTGCAAAGGTGATGAATGTGATGGGCCCAATACGATGGA
CTTGCTGGAAATGGCGATTAATGATTTGGTGGCTGAAGGGCGTCTGGGAGAGGAGAAAT
TGGATTCTTTTAACGTTCCAATCTACACAGCCAGCGTTGAAGAGGTTAAATGTATGGTGG
AAGAAGAAGGCTCTTTCGAAATACTCTATCTTCAGACATTTAAACTGCGGTATGATGCCG
GTTTTTCCATTGACGATGACTGCCAAGTTAGAAGCCATTCGCCCGTCTACAGCGATGAAC
ATGCACGTGCCGCTCATGTTGCCAGCTTAATTCGCTCAGTGTACGAACCGATCCTGGCGT
CGCATTTCGGCGAGGCTATAATACCGGATATATTTCACCGTTTTGCAACTAACGCGGCCA
AGGTGATTCGCTTGGGGAAAGGGTTCTACAACAACCTTATTATATCACTTGCTAAGAAAC
CTGAAAAGAGTGACATCTAA
Polypeptide sequence encoded by the polynucleotide of SEQ ID NO: 47:
(SEQ ID NO: 48)
MELQEVLHMNGGEGEASYAKNSSFNQLVLAKVKPVLEQCVRELLRANLPNINKCIKVADLG
CASGPNTLLTVWDTVQSIDKVKQEMKNELERPTIQVFLTDLFQNDFNSVFMLLPSFYRKLEK
ENGRKIGSCLIAAMPGSFHGRLFPEESMHFLHSSYSLQFLSQVPSGLVTELGITANKRSIYSSK
ASPPPVQKAYLDQFTKDFTTFLRMRSEELLSRGRMLLTCICKGDECDGPNTMDLLEMAINDL
VAEGRLGEEKLDSFNVPIYTASVEEVKCMVEEEGSFEILYLQTFKLRYDAGFSIDDDCQVRSH
SPVYSDEHARAAHVASLIRSVYEPILASHFGEAIIPDIFHRFATNAAKVIRLGKGFYNNLIISLA
KKPEKSDI
DXMT_23:
(SEQ ID NO: 49)
ATGGAACTCCAGGAAGTTCTGCACATGAACGGTGGCGAAGGTGATGCTTCGTATGCTAA
AAATTCAAGCTTTAATCAGTTGGTTCTGGCTAAAGTGAAGCCGGTACTTGAACAATGCGT
GGGAGAGCTCTTACGCGCAAATCTCCCCAATATTAACAAATGTATAAAAGTTGCGGATTT
AGGGTGTGCCTCCGGCCCGAACACCTTGTTAACCGTCCGTGATATCGTACAGTCAATCGA
TAAAGTTAGACAGGAAATGAAGAATGAATTGGAACGCCCCACCATCCAAGTCTTTCTTA
CGGACCTGTTCCAGAACGATTTCAACAGCGTGTTCATGCTGTTACCGTCATTCTACCGCA
AATTAGAAAAGGAGAACGGTCGGAAGATCGGTTCTTGTCTGATAGCAGCAATGCCCGGC
TCTTTTCATGGTCGTCTTTTCCCCGAAGAATCGATGCATTTCCTCCACAGCAGCTATTCAC
TGCAGTTCTTATCACAAGTTCCGTCAGGACTGGTTACTGAGTTGGGTATAACTGCGAACA
AACGCTCTATCTATAGCTCAAAAGCAAGTCCACCGCCAGTGCAAAAGGCCTACTTGGATC
AGTTTACTAAAGACTTTACGACCTTCCTGCGCATACGGAGTGAAGAACTCCTTTCTCGTG
GTAGAATGTTATTGACATGCATCTGCAAAGGCGATGAGTTCGATGGACCAAACACTATG
GATCTGTTAGAAATGGCAATAAACGATCTCGTCGTGGAAGGCCACCTTGAGGAAGAAAA
ACTGGACAGCTTCAACGTCCCCATTTATGCTGCTTCGGTTGAAGAGCTGAAGTGTATTGT
GGAAGAAGAGGGTTCATTTGAGATCTTATATTTAGAAACCTTCAAACTCCGCTACGATGC
GGGTTTTTCGATTGATGATGACTGCCAGGTACGTAGTCATAGTCCAGAATATTCCGATGA
GCATGCAAGAGCAGCGCATGTCGCGTCATTACTGCGGTCCGTATATGAACCGATCTTAGC
GAACCACTTTGGCGAAGCGATAATCCCTGATATCTTTCATCGTTTTGCGACTAATGCAGC
GAAAGTGATTAGATTGGGGAAGGGTTTTTACAATAACCTCATAATTAGCCTGGCTAAAA
AGCCCGAGAAGAGCGATATCTAA
Polypeptide sequence encoded by the polynucleotide of SEQ ID NO: 49:
(SEQ ID NO: 50)
MELQEVLHMNGGEGDASYAKNSSFNQLVLAKVKPVLEQCVGELLRANLPNINKCIKVADLG
CASGPNTLLTVRDIVQSIDKVRQEMKNELERPTIQVFLTDLFQNDFNSVFMLLPSFYRKLEKE
NGRKIGSCLIAAMPGSFHGRLFPEESMHFLHSSYSLQFLSQVPSGLVTELGITANKRSIYSSKAS
PPPVQKAYLDQFTKDFTTFLRIRSEELLSRGRMLLTCICKGDEFDGPNTMDLLEMAINDLVVE
GHLEEEKLDSFNVPIYAASVEELKCIVEEEGSFEILYLETFKLRYDAGFSIDDDCQVRSHSPEYS
DEHARAAHVASLLRSVYEPILANHFGEAIIPDIFHRFATNAAKVIRLGKGFYNNLIISLAKKPE
KSDI
DXMT_25:
(SEQ ID NO: 51)
CTGCAAGAAGTCCTTCACATGAACGGTGGCGAAGGGGATACTAGCTACGCTAAAAACTC
CAGTTACAATCTGGTGCTGACAAAAGTTAAACCTGTGCTGGAACAATGCATTAGAGAGC
TCCTGAGAGCGAACCTTCCGAATATTAATAAATGCATTAAAGTAGCCGACTTGGGTTGCG
CTTCAGGCCCTAACACGCTTCTCACTGTTCGCGATATTGTCCAGTCCATAGATAAGGTTG
GTCAGGAGGAGAAGAATGAATTGGAGCATCCTACCATTCAGATCTTCTTGAATGACCTTT
TTCAAAATGATTTCAACAGCGTATTCAAGCTGCTCCCTAGTTTTTATCGGAAACTTGAGA
AAGAGAACGGGAGAAAAATAGGCAGTTGTTTAATCTGGGCAATGCCTGGCAGCTTTTAC
AGCCGGCTTTTCCCAGAAGAAAGTATGCACTTTCTTCACTCATGCTATTGTCTGCAGTGGT
TAAGTCAAGTCCCTAGTGGCTTAGTTACAGAGCTGGGTATTAGTGCCAATAAAGGCATAA
TATACTCTTCTAAGGCTTCTCCACCACCGGTGCAGAAGGCCTATCTGGATCAATTCACCA
AAGATTTTACCACATTTCTGCGTATTCACTCTGAAGAACTCCTTTCCAGAGGACGTATGCT
GTTAACTTGTATCTGCAAAGGAGATGAAAGTGACGGGTTGAACACAATAGACCTTCTGG
AGCGGGCAATTAACGACCTCGTAGTAGAAGGTTTGTTAGAAGAGGAAAAGTTAGATTCA
TTCAACTTGCCATTATATACTCCATCGCTCGAAGTGGTCAAATGCATGGTGGAAGAATAA
Polypeptide sequence encoded by the polynucleotide of SEQ ID NO: 51:
(SEQ ID NO: 52)
MQEVLHMNGGEGDTSYAKNSSYNLVLTKVKPVLEQCIRELLRANLPNINKCIKVADLGCAS
GPNTLLTVRDIVQSIDKVGQEEKNELEHPTIQIFLNDLFQNDFNSVFKLLPSFYRKLEKENGRK
IGSCLIWAMPGSFYSRLFPEESMHFLHSCYCLQWLSQVPSGLVTELGISANKGIIYSSKASPPPV
QKAYLDQFTKDFTTFLRIHSEELLSRGRMLLTCICKGDESDGLNTIDLLERAINDLVVEGLLEE
EKLDSFNLPLYTPSLEVVKCMVEE
DXMT_26:
(SEQ ID NO: 53)
CTGCAGGAAGTATTGCACATGAATGAAGGTGAGGGTGATACAAGTTATGCCAAGAATGC
ATCTTATAATCTCGCTTTGGCGAAAGTGAAGCCATTTCTTGAGCAGTGCATTCGTGAATT
GCTCCGTGCAAATCTTCCGAATATCAATAAGTGCATTAAAGTCGCCGATCTTGGATGTGC
TTCAGGACCAAATACTCTGCTTACAGTGCGGGACATAGTACAATCCATTGACAAAGTAG
GACAGGAGGAGAAGAATGAGCTGGAACGCCCAACGATACAGATATTCCTTAATGATCTG
TTTCAGAACGATTTCAATAGCGTTTTTAAGCTGTTACCCTCATTCTACAGAAAACTGGAG
AAGGAAAACGGGCGCAAAATTGGGTCTTGCTTAATAAGTGCCATGCCTGGTTCCTTCTAT
GGTCGCTTATTTCCTGAAGAAAGTATGCACTTTCTGCATAGTTGCTATTCGGTTCATTGGC
TTTCTCAGGTTCCAAGCGGACTTGTTATTGAATTGGGTATCGGCGCTAACAAGGGTTCCA
TTTATAGTAGTAAAGGATGTCGTCCGCCAGTTCAGAAAGCTTATCTTGATCAATTCACCA
AAGACTTTACGACGTTCTTGCGTATTCATTCCAAGGAGCTGTTTTCTCGGGGACGTATGCT
GCTGACGTGTATATGCAAAGTGGATGAATTCGATGAACCCAATCCGCTGGACCTGCTGG
ATATGGCAATAAATGACCTCATCGTAGAAGGCAGATTAGGGGAAGAGAAACTTGATTCA
TTTAATGTCCCCATTTATACCGCTAGCGTCGAAGAAGTAAAATGCATGGTTGAAGAATAA
Polypeptide sequence encoded by the polynucleotide of SEQ ID NO: 53:
(SEQ ID NO: 54)
MQEVLHMNEGEGDTSYAKNASYNLALAKVKPFLEQCIRELLRANLPNINKCIKVADLGCAS
GPNTLLTVRDIVQSIDKVGQEEKNELERPTIQIFLNDLFQNDFNSVFKLLPSFYRKLEKENGRK
IGSCLISAMPGSFYGRLFPEESMHFLHSCYSVHWLSQVPSGLVIELGIGANKGSIYSSKGCRPP
VQKAYLDQFTKDFTTFLRIHSKELFSRGRMLLTCICKVDEFDEPNPLDLLDMAINDLIVEGRL
GEEKLDSFNVPIYTASVEEVKCMVEE
DXMT_27:
(SEQ ID NO: 55)
ATGGAAGTAAAGGAAGCGCTGTTCATGAATAGAGGTGAAGGCGAATCCTCATACGCTCA
AAACAGTAGTTTTACTCAGAAGGTTGCGTCTATGACTATGCCGGTATTGGAGAATGCCGT
AGAAACCTTATTTAGCAAGGATTTCCATCTTTTTCAGGCACTGAATGCCGCGGATCTGGG
TTGCGCTACAAGTCCAAATACATTCACGGTGATTTCGACCATTAAACGTATGATGGAGAA
GAAATGCCGCGAATTGAACTGTCAGACGTTAGAACTGCAAGTTTACTTGAATGACCTGCC
CGGAAATGACTTCAACACTCTGTTCAAAGGACTTTTGTCTAAGGTAGTGGTGGGAAATAA
GTGCGAAGAAGTCTCATGCTATGTGATGGGCGTGCCGGGTAGCTTCCATGGTCGGCTTTT
TCCTAGAAATAGCTTACACCTTGTCCACTCATGCTACTCAGCCCACTGGTTATCTCAAGCT
CCTAAAGGCCTGACGTCTCGGGAAGGTTTACCGCTTAACAAAGGAAAGATTTACATTTCA
AAACGCAGCCCGCCGGTGGTACGGGAAGCCTATTTGAGTCAGTTTCATGATGATTTTACG
ATGTTTCTTAATGCACGTTCCCAGGAAGTGGTGCCGCATGGGTGTATGGTGCTCATCCTG
CCTAGTAGACAATCCAGCGATCCAAGTTCTATGGAATCCTGCTTCACCTGGGAACTTTTG
GCAATTGCTATTGCCGAACTCGTATCACAAGGCCTTATTGATGAGGATAAACTGGACACC
TTCAATGTACCATCATACTTTCCCAGTTTAGAAGAAGTAAAGGATATCGTAGAGCGCGAT
GGCTCTTTCACTATCGATCACATGGAGGGTTTCGAGCTGGATACACTGCAGATGCAGGAA
AATGACAAGTGGATACGCGGAGAGAAATTAGCCAAAGCAGTTAGAGCGTTCACGGAGCC
TATCATTTCAAACCAGTTCGGCCATGAGATAATGGATAAGCTGTATGACAAATTTACACA
TATAGTCGCGAGCGATCTGGAAGGGAAAATCCCCAAATCAACGTCCATAGTTTTGGTGCT
GAGCAAGATTGTCGGCTAA
Polypeptide sequence encoded by the polynucleotide of SEQ ID NO: 55:
(SEQ ID NO: 56)
MEVKEALFMNRGEGESSYAQNSSFTQKVASMTMPVLENAVETLFSKDFHLFQALNAADLGC
ATSPNTFTVISTIKRMMEKKCRELNCQTLELQVYLNDLPGNDFNTLFKGLLSKVVVGNKCEE
VSCYVMGVPGSFHGRLFPRNSLHLVHSCYSAHWLSQAPKGLTSREGLPLNKGKIYISKRSPPV
VREAYLSQFHDDFTMFLNARSQEVVPHGCMVLILPSRQSSDPSSMESCFTWELLAIAIAELVS
QGLIDEDKLDTFNVPSYFPSLEEVKDIVERDGSFTIDHMEGFELDTLQMQENDKWIRGEKLAK
AVRAFTEPIISNQFGHEIMDKLYDKFTHIVASDLEGKIPKSTSIVLVLSKIVG
DXMT_28:
(SEQ ID NO: 57)
ATGGAGGTGAAAGAGGCGCTGTTCATGAATCGGGGAGAAGGTGAGAATAGTTATGCACA
AAACTCCTCCTTTACACAGAAGGTTGCGAGTATGACCATGCCGGTACTCGAAAACGCTGT
GGAAACCCTTTTCTCTAAAGATTTCCATTTGCTTCAGGCGCTGAACGTAGTAGATCTGGG
ATGCGCTACGAGTCCGAATACTTTCACGGTAATCAGCACCATCAAGCGGATGATGGAAA
AGAAGTGCCGGGAACTCAATTGTCAGACACTTGAACTGCAGGTTTATCTCAATGATCTGC
CAGGTAATGACTTCAATACACTGTTTAAAGGCCTTTTGTCTAAAGTTGTAGTCGGCAACA
AGTGCGAAGAAGTGTCATGCTATGTTATGGGCGTTCCTGGGTCATTTCACGGTCGTTTAT
TTCCGAGAAATTCATTGCGCTTGGTTCACTCTTGCTATTCTGCTCACTGGTTATCTCAGGC
GCCCAAGGGGTTGACATCACGTGAAGGTTTAGCATTGAACCGCCGCAAAATATACATTTC
CAAAACCAGCCCTCTGGTAGTGCGTGAAGCGTATCTTTCCCAGTTTCACGAAGATTTCAC
TATGTTCTTAAATGCCCGTAGTCAGGAAGTAGTTCCCAATGGCTGCATGGTCCTGATACT
TCCGGGGCGTCAGTCTTCAAATCCGTCATCAATGGAGAGCTGCTTTACCTGGGAACTGTT
AGCGATAGCCATAGGAGAGCTCGTGAGCCAAGGCCTCATCGATGAGGACAAGCTCGATA
CATTTAACGTGCCGAGTTATTTCCCGAGCTTAGAAGAGGTTAAGGACATAGTCGAGAGA
GATGGTAGTTTTACAATAGATCACATGGTTGGCTTCGAACTGGATACGCCCCAGATGCAA
GAGAATGATAAATGGGTACGTGTAGAAAAGTTAGCTAAGGCGGTTAGAGCTTTTACTGA
GCCAATCATAAGTAATCAGTTTGGTCACGAAATTATGGACAAACTCTACGACAAATTCAC
TTACATCGTGGTAAGTGATTTGGAAGGGAAGATCCCGAAAACTACGTCGATAGTACTGG
TGCTGTCGAAAATTATCGGATAA
Polypeptide sequence encoded by the polynucleotide of SEQ ID NO: 57:
(SEQ ID NO: 58)
MEVKEALFMNRGEGENSYAQNSSFTQKVASMTMPVLENAVETLFSKDFHLLQALNVVDLG
CATSPNTFTVISTIKRMMEKKCRELNCQTLELQVYLNDLPGNDFNTLFKGLLSKVVVGNKCE
EVSCYVMGVPGSFHGRLFPRNSLRLVHSCYSAHWLSQAPKGLTSREGLALNRRKIYISKTSPL
VVREAYLSQFHEDFTMFLNARSQEVVPNGCMVLILPGRQSSNPSSMESCFTWELLAIAIGELV
SQGLIDEDKLDTFNVPSYFPSLEEVKDIVERDGSFTIDHMVGFELDTPQMQENDKWVRVEKL
AKAVRAFTEPIISNQFGHEIMDKLYDKFTYIVVSDLEGKIPKTTSIVLVLSKIIG
DXMT_29:
(SEQ ID NO: 59)
ATGAAAGAAGTAAAAGAAGCTCTGTTTATGAACAAAGGCGAAGGTGAATCCAGTTACGC
TCAGAATAGCTCGTTCACTCAGACCGTAACGTCCATGACCATGCCGGTGTTAGAGAATGC
CGTTGAGACATTATTCTCCAAGGATTTTCACTTGCTGCAAGCTCTTAATGCAGTAGATTTA
GGATGCGCAGCTGGACCAACTACATTTACAGTGATTTCGACTATCAAACGTATGATGGAG
AAGAAATGTCGCGAACTCAATTGTCAAACTTTAGAACTTCAGGTATACCTGAACGATTTA
CCTGGTAACGACTTTAATACACTGTTTAAAGGTCTTCCGTCTAAAGTCGTCGGTAATAAA
TGCGAAGAAGTCAGCTGCTACGTAGTAGGTGTTCCAGGTTCATTTCATGGTCGGTTATTT
CCTCGTAATTCCCTTCACTTGGTGCATTCATGTTACAGTGTTCATTGGTTGACTCAGGCTC
CGAAAGGTTTGACCAGTAAGGAGGGGTTAGCGCTCAACAAAGGTAAGATCTATATTAGT
AAGACATCCCCTCCAGTGGTTAGAGAGGCATACTTGTCTCAGTTTCACGAGGATTTTACT
ATGTTTCTGAACTCACGGAGCCAGGAAGTTGTTCCAAATGGCTGCATGGTATTGATTCTG
AGAGGTCGTCTTTCGAGTGATCCTAGCGATATGGGTTCATGTTTTACTTGGGAACTGCTG
GCCGTGGCGATCGCAGAACTCGTGAGCCAAGGTTTAATCGATGAAGACAAGCTGGACAC
CTTTAACGTGCCGTCATATTTCCCTTCCCTTGAAGAGGTGAAAGACATAGTTGAACGCAA
TGGCTCTTTCACTATTGACCACATGGAGGGCTTCGAATTGGATTCACCCGAGATGCAGGA
AAATGATAAATGGGTTCGGGGCGAAAAATTTGCTACAGTTGCGCGGGCGTTCACGGAAC
CGATTATTTCAAATCAGTTTGGGCACGAAATCATGGATAAACTTTATGAGAAATTTACAC
ACATCGTGGTGAGTGATTTCGAAGCCAAAATACCGAAAATTACTTCTATAATCTTGGTTC
TCAGTAAAATTGTGGGCTAA
Polypeptide sequence encoded by the polynucleotide of SEQ ID NO: 59:
(SEQ ID NO: 60)
MKEVKEALFMNKGEGESSYAQNSSFTQTVTSMTMPVLENAVETLFSKDFHLLQALNAVDLG
CAAGPTTFTVISTIKRMMEKKCRELNCQTLELQVYLNDLPGNDFNTLFKGLPSKVVGNKCEE
VSCYVVGVPGSFHGRLFPRNSLHLVHSCYSVHWLTQAPKGLTSKEGLALNKGKIYISKTSPPV
VREAYLSQFHEDFTMFLNSRSQEVVPNGCMVLILRGRLSSDPSDMGSCFTWELLAVAIAELV
SQGLIDEDKLDTFNVPSYFPSLEEVKDIVERNGSFTIDHMEGFELDSPEMQENDKWVRGEKFA
TVARAFTEPIISNQFGHEIMDKLYEKFTHIVVSDFEAKIPKITSIILVLSKIVG
MXMT_30:
(SEQ ID NO: 61)
ATGGAGGTAAAAGAAGCCTTGTTTATGAACAAGGGAGAGGGCGAAAGTTCCTACGCTCA
GAGTTCTAGTTTTACGGAAACAGTTACTAGTATGACTATGCCAGTGCTTGAGAATGCTGT
TGAAACTCTGTTCTCAAAAGATTTCCATCTTTTACAAGCATTGAACGCAGCCGACCTTGG
CTGTGCAGCAGGTCCTACCACGTTTACGGTAATCAGCACCATAAAACGTATGATGGAGA
AGAAGTGCCGGGAGCTGAATTGTCAGACGTTAGAACTCCAGGTATACCTGAACGACTTG
CCCGGCAATGACTTCAACACCCTGTTTAAAGGGTTGTCGAGTAAGGTGGTGGGCAACAA
GTGCGAAGAAGTGCCATGTTATGTTGTAGGCGTACCCGGGAGCTTTCATGGTAGACTTTT
TCCCCGTAACAGCCTGCACTTAGTTCACTCGTGTTATTCAGTTCACTGGTTGACTCAGGCA
CCTAAAGGACTGACTAGTAAAGAGGGTCTGGCCCTGAATAAAGGTAAAATCTACATAAG
CAAGACCTCTCCGCCAGTAGTGCGGGAAGCCTACTTGTCACAGTTTCATGAGGATTTTAC
AATGTTTCTTAATTCGCGCTCGCAAGAAGTTGTTCCAAATGGGTGTATGGTATTAATTTTA
CGTGGACGTCTGTCTTCCGATCCGAGCGACATGGAGTCATGTTTTACTTGGGAACTCTTA
GCAGTGGCCATAGCTGAGCTGGTGTCACAGGGTCTGATTGATGAGGATAAGCTCGACAC
ATTTAACGTACCGTCCTATTTTCCGAGTCTGGAGGAAGTTAAAGATATAGTCGAGCGCAA
TGGCTCCTTCACGATAGATCACATGGAGGGTTTCGAACTTGACAGTCCACAGATGCAGGA
AAATGACAAATGGGTTCGTGGTGAAAAGTTTGCGACAGTGGCACGCGCTTTTACAGAGC
CCATTATTAGCAATCAGTTTGGCCATGAAATTATGGACAAACTCTATGAGAAATTCACCC
ACATTGTAGTGTCAGACTTAGAAGCTAAAATCCCGAAAATAACCTCAATCATCCTGGTTT
TATCGAAAATTGTAGGCTAA
Polypeptide sequence encoded by the polynucleotide of SEQ ID NO: 61:
(SEQ ID NO: 62)
MEVKEALFMNKGEGESSYAQSSSFTETVTSMTMPVLENAVETLFSKDFHLLQALNAADLGC
AAGPTTFTVISTIKRMMEKKCRELNCQTLELQVYLNDLPGNDFNTLFKGLSSKVVGNKCEEV
PCYVVGVPGSFHGRLFPRNSLHLVHSCYSVHWLTQAPKGLTSKEGLALNKGKIYISKTSPPVV
REAYLSQFHEDFTMFLNSRSQEVVPNGCMVLILRGRLSSDPSDMESCFTWELLAVAIAELVSQ
GLIDEDKLDTFNVPSYFPSLEEVKDIVERNGSFTIDHMEGFELDSPQMQENDKWVRGEKFAT
VARAFTEPIISNQFGHEIMDKLYEKFTHIVVSDLEAKIPKITSIILVLSKIVG
MXMT_31:
(SEQ ID NO: 63)
ATGATGGAAGTGAAAGAAGCCCTGTTTATGAATCGGGGTGAAGGGGAAAGCAGCTACGC
GCAAAACTCGTCATTTACTCAAAAAGTTGCATCATTAACCATGCCAGTTCTTGAGAATGC
CGTGGAGACTCTGTTCTCAAAAGATTTCCACCTTCTGCAGGCATTGAATGCCGCCGATCT
GGGTTGTGCCGCAGGGCCGAACACATTTACCGTTATATTCACAATTAAACGCATGATGGA
GAAGAAATGTAGAGAACTGAATTGCCAGACCCTGGAGCTGCAAGTGTATCTCAACGACC
TGCCTGGGAACGATTTTAACACATTGTTTAAGGGTCTGTCGTCAAAAGTCGTAGGTAACA
AATGCGAAGAGGTATCGTACTATGTCATGGGAGTGCCAGGTTCCTTTCATGGCCGCTTGT
TTCCACGCAATAGTCTGCATCTGGTACACTCGAGTTATAGCGTACATTGGTTGTCTCAAG
CACCTAAGGGTTTGAGATCCCGCGAAGGCTTGGCTCTGAATAAGGGCAAAATTTACATAT
CGAAAACGTCTCCTCCGGTTGTCCGGGAAGCCTACTTATCACAATTCCACGAGGATTTTA
CTATGTTCCTTAATGCCCGTAGCCAAGAAGTAGTGCCTAATGGATGTATGGTGCTTATCC
TTCACGGTCGTAAATCATCAGACCCGTCGAATATGGAAAGTTGCTTTACGTGGGAACTCT
TAGCCATCGCAATTAGCGAGCTTGTGTCACAGGGCTTGATCGATGAGGATAAACTCGATA
CCTTCAACGTGCCATATTATACGCCTAGCCTTGAGGAGATGAAAGATATCGTAGAACGTG
AAGGATCATTTACAATTGACCATATAGAGGGCTTTGAGCTTGATTCCCCGCACATGCAAG
AAAAAGACAAGTGGGCGGGTAGAGAAAAGCTTGCAAAAGCCATACGCGCTTTTACCGAG
CCGATTATCTCCAATCAGTTCGGCCATGAGATTATGGACAAGTTATATGACAAGTTCACA
CATATAGTTGTCTCTGACTTAGAGGCTAAAATCCCAAAAACCACCTCAATCATCCTGGTC
TTGTCTAAAATTGTTGGGTAA
Polypeptide sequence encoded by the polynucleotide of SEQ ID NO: 63:
(SEQ ID NO: 64)
MMEVKEALFMNRGEGESSYAQNSSFTQKVASLTMPVLENAVETLFSKDFHLLQALNAADLG
CAAGPNTFTVIFTIKRMMEKKCRELNCQTLELQVYLNDLPGNDFNTLFKGLSSKVVGNKCEE
VSYYVMGVPGSFHGRLFPRNSLHLVHSSYSVHWLSQAPKGLRSREGLALNKGKIYISKTSPP
VVREAYLSQFHEDFTMFLNARSQEVVPNGCMVLILHGRKSSDPSNMESCFTWELLAIAISELV
SQGLIDEDKLDTFNVPYYTPSLEEMKDIVEREGSFTIDHIEGFELDSPHMQEKDKWAGREKLA
KAIRAFTEPIISNQFGHEIMDKLYDKFTHIVVSDLEAKIPKTTSIILVLSKIVG
MXMT_32:
(SEQ ID NO: 65)
ATGAATCGGGGCGAAGGAGAATCTAGCGATGCCCAGAATAGCTCCTTCACACAGAAAGG
CGCATCCATGACTATGCCGGTGCTCGAGAATGCGGTGGAAACCCTGTTCTCGAAAGATTT
CCATTTGCTGCAAGCGTTAAATGCCGCTGATTTTGGATGTGCTGCCGGCCCGAATACTGT
CATTTCGACAATAAAAAGAATGATGGAGAAAAAGTGTCGCGAGCTGAATTGTCAGACGC
TCGAGTTGCAGGTTTACCTGAACGATCTCCCGGGTAATGACTTCAACACCCTGTTCAAGG
GTCTTAGCTCAAAAATTGTTGTGGGAAACAAGTGCGAGGAGGTAAGTTGTTATGCGATG
GGCGTTCCGGGCTCTTTCCACGGAAGATTATTTCCCCGTAACTCGCTCCATCTGGTGCATT
CAAGTTATTCCGTCCATTGGCTCTCACAGGCACCGAAAGGCTTAAGATCACGCGAGGGA
CTGGCCCTCAACAAAGGCAAAATTTACATATCAAAAACATCACCTCCGGTTGTTCGCGAA
GCGTATTTAAGCCAGTTCCATGAAGACTTTACGATGTTCTTAAACGCCAGAAGTCAAGAG
GTGGTACCGAACGGGTGCATGGTGTTAATTTTGCATGGCAGAAAGTCTTCCGACCCGTCT
AACATGGAATCGTGTTTCACTTGGGAACTGTTAGCTATCGCGATCGCGGAGCTGGTATCA
CAAGGGCTCATTGACGAGGACAAGCTTGACACGTTTAATGTGCCATATTACACGCCTAGT
CTGGAAGAAATGAAAGATATTGTAGAACGCGAGGGGTCTTTTACCATTGACCACATTGA
GGGTGTGGAATTAGATAGCCCCCACATGCAAGAAAAAGATAAATGGGCAGGCAGAGAG
AAGTTAGCCAAAGCTATTCGCGCGTTTACTGAACCAATCATCTCAAACCAGTTCGGTCAT
GAGATCATGGATAAACTGTACGATAAGTTTACGCACATTGTTGTAAGTGATCTGGAGGCA
AAAATACCAAAAACTACTTCGATTATCCTCGTCCTGTCAAAAATAGTTGGCTAA
Polypeptide sequence encoded by the polynucleotide of SEQ ID NO: 65:
(SEQ ID NO: 66)
MNRGEGESSDAQNSSFTQKGASMTMPVLENAVETLFSKDFHLLQALNAADFGCAAGPNTVI
STIKRMMEKKCRELNCQTLELQVYLNDLPGNDFNTLFKGLSSKIVVGNKCEEVSCYAMGVP
GSFHGRLFPRNSLHLVHSSYSVHWLSQAPKGLRSREGLALNKGKIYISKTSPPVVREAYLSQF
HEDFTMFLNARSQEVVPNGCMVLILHGRKSSDPSNMESCFTWELLAIAIAELVSQGLIDEDKL
DTFNVPYYTPSLEEMKDIVEREGSFTIDHIEGVELDSPHMQEKDKWAGREKLAKAIRAFTEPII
SNQFGHEIMDKLYDKFTHIVVSDLEAKIPKTTSIILVLSKIVG
MXMT_33:
(SEQ ID NO: 67)
ATGATGGAAGTTAAGGAAGCACTGTTCATGAATGGTGGTGAGGGCGAATCCTCTTATGC
GCAAAATTCCAGTTTCACACAGAAAGTGGCGAGTATGACAATTCCTGTGTTGGAAATAG
CTGTGGAGACAATATTTTCTAAGGATTTCCATCTTTTACAAGCACTTAATGCCGCTGATCT
GGGGTGCGCAGCCGGACCAAATACATTTACGGTTATTTCAACTATTAAGCGCATGATGGA
AAAGAAATGCCGGGAGCTCAACTGCGAAACTTTGGAATTACAAGTATATCTTAATGATCT
TCCCGGCAATGACTTCAATACGTTGTTTAAAGGCCTGAGCAGTACGGATGTGGTTGGAAA
TAAGTGCGAAGAAGTTTCCTGTTATGTTATGGGTGTCCCAGGCTCGTTTCATGGTCGTCTG
TTTCCGCGTAACTCATTGCATTTGGTACATAGTTCTTATAGCGTTCATTGGTTATCCCAGG
CCCCAAAGGGCTTACGGTCTAGAGAAGGCCTGGCATTGAACAAAGGTAAAATCTACATT
AGTAAGACCTCACCACCTGTTGTTAGAGAGGCGTATCTGAGTCAGTTCCATGAAGATTTC
ACAATGTTCTTAAACGCCCGTAGTCAAGAAGTGGTTCCTAATGGGTGTATGGTCCTCATT
CTCCATGGAAGAAAATCATCCGATCCCTCCAAAATTGAATGCTGCTTTACTTGGGAATTA
TTGGCCATTGCTATCGCCGAACTTGTTTCCCAGGGGCTGATCGACGAGGATAAATTAGAC
ACCTTCAACGTGCCATACTACACGCCATCTCTGGAAGAGGTTAAGGACATTGTAGAACGC
GAGGGCTCGTTCACTATCGATCACATGGAGGGCCTGGAATTAGACTCTCCACAAATGCA
GGAAAAAGATAAATGGGTACGGGGTGAAAAACTGGCGAAAGCGGTACGCGCATTCACG
GAACCAATAATATCCAACCAGTTTGGTCACGAAATTATGGACAAGCTTTATGACAAATTC
ACTCACATCGTAGTATCAGACCTTGAAGCCAAAAAGCCTAAAACTACCAGTATAATTTTG
GTGCTGAGCAAAATTGTTGGCTAA
Polypeptide sequence encoded by the polynucleotide of SEQ ID NO: 67:
(SEQ ID NO: 68)
MMEVKEALFMNGGEGESSYAQNSSFTQKVASMTIPVLEIAVETIFSKDFHLLQALNAADLGC
AAGPNTFTVISTIKRMMEKKCRELNCETLELQVYLNDLPGNDFNTLFKGLSSTDVVGNKCEE
VSCYVMGVPGSFHGRLFPRNSLHLVHSSYSVHWLSQAPKGLRSREGLALNKGKIYISKTSPPV
VREAYLSQFHEDFTMFLNARSQEVVPNGCMVLILHGRKSSDPSKIECCFTWELLAIAIAELVS
QGLIDEDKLDTFNVPYYTPSLEEVKDIVEREGSFTIDHMEGLELDSPQMQEKDKWVRGEKLA
KAVRAFTEPIISNQFGHEIMDKLYDKFTHIVVSDLEAKKPKTTSIILVLSKIVG
MXMT_34:
(SEQ ID NO: 69)
ATGATGGAGGTGAAAGAGGCCCTTTTTATGAATGGTGGCGAGGGAGAATCGTCTTACGC
CCAGAACAGTTCATTTACGCAAAAGGTTGCCTCTATGACTATGCCTGTGCTTGAAATTGC
AGTTGAAACGTTACTGAGCAAAGACTTCCATTTACTGCAGGCCCTGAACGTAGCGGATCT
GGGGTGTGCGGCCGGTCCGAATACATTTACCGTAATATCCACCATTAAACGGATGATGG
AAAAGAAATGTCGCGAACTGAATTGTCAGACGCTGGAATTGCAAGTTTACCTTAACGAT
CTGCCGGGGAATGATTTTAACACACTGTTCAAAGGCCTCAGCTCAAAGGTTGTTGTAGGT
AACAAATGCGAGGAAGTTAGCTGTTATGCTATGGGCGTCCCAGGTAGCTTCCACGGTAG
ATTATTTCCACGGAATTCGTTACACTTGGTCCACTCCAGCTATTCAGTGCACTGGCTGTCC
CAAGCACCTAAAGGCCTCCGGTCAAGAGAAGGGCTGGCGCTTAACAAAGGTAAAATCTA
CATAAGTAAGACATCTCCCCCAGTTGTTAGAGAAGCGTATTTGTCCCAGTTTCATGAAGA
CTTTACAATGTTCTTGAATGCTCGCTCCCAGGAGGTTGTACCCAATGGATGCATGGTCCT
GATCTTGCACGGAAGAAAATCAAGTGATCCTAGCAACATGGAGTCATGCTTCACATGGG
AACTGTTAGCGATTGCGATTGCCGGGCTTGTCTCACAGGGCTTAATAGACGAAGACAAA
CTCGATACGTTTAACGTGCCCTATTACACCCCGAGCCTCGAAGAAGTAAAAGACATAGTG
GAACGCGAAGGCAGTTTCACTATCGATCACATGGAAGGATTAGAATTAGATAATCCTCA
CATGCAGGAAAAAGACAAATGGGTACGTGGTGCTAAACTGGCTAAAGCTGTGCGCGCGT
TTACGGAGCCGATTATCAGCAAACAATTCGGCCATGAGATTATGAATAAGCTGTATGATA
AATTCACACATATTGTCGTTAGTGAGCTCGAGGCCAAGAAACCTAAGACTACCAGTATA
ATTCTTGTCTTGAGCAAAATAGTTGGGTAA
Polypeptide sequence encoded by the polynucleotide of SEQ ID NO: 69:
(SEQ ID NO: 70)
MMEVKEALFMNGGEGESSYAQNSSFTQKVASMTMPVLEIAVETLLSKDFHLLQALNVADLG
CAAGPNTFTVISTIKRMMEKKCRELNCQTLELQVYLNDLPGNDFNTLFKGLSSKVVVGNKCE
EVSCYAMGVPGSFHGRLFPRNSLHLVHSSYSVHWLSQAPKGLRSREGLALNKGKIYISKTSPP
VVREAYLSQFHEDFTMFLNARSQEVVPNGCMVLILHGRKSSDPSNMESCFTWELLAIAIAGL
VSQGLIDEDKLDTFNVPYYTPSLEEVKDIVEREGSFTIDHMEGLELDNPHMQEKDKWVRGAK
LAKAVRAFTEPIISKQFGHEIMNKLYDKFTHIVVSELEAKKPKTTSIILVLSKIVG
MXMT_35:
(SEQ ID NO: 71)
ATGGGCAAAGTTAACGAAGTTTTATTCATGAACCGCGGAGAGGGGGAGATCTCATACGC
ACAGAATTCAGCGTTTACCCAAAAAGTTGCGAGCATGGCAATGCCGGCCTTGGAGAACG
CGGTTGAGACTTTGTTTAGCAAAGACTTTCATTTACTTCAGGCACTTACTGCTGCTGATCT
TGGCTGTGCTGCCGGGCCGAATACCTTCGCCGTTATTTCTACGATTAAGCGGATGATGGA
GAAGAAATGTAGAGAATTATACTGCCAAACGCTCGAACTGCAGGTATATTTGAACGACC
TGTTCGGAAATGATTTCAATACCCTCTTCAAAGGCCTCTCATCAGAAGTTGTCGGCAATA
AGTGCGAAGAAGTATCCTGCTATGTTATGGGTGTTCCTGGGTCATTCCATGGACGTTTAT
TTCCCCGCAATTCATTGCATTTAGTTCACTCAAGCTACTCAGTGCATTGGCTGACCCAGGC
CCCAAAAGGCTTAACAAGTCGGGAAGGCCTCGCATTGAACAAAGGCAAGATATATATCT
CTAAAACATCACCGCCAGTTGTGAAAGAAGCGTACCTGTCGCAGTTTCACGAAGATTTTA
CTATGTTCTTAAATGCGCGCTCTCAGGAAGTAGTGCCCAATGGTTGCATGGTTTTGATCCT
GCATGGACGTCAAAGTTCCGATCCCTCAGAAATGGAGTCCTGCTTTACTTGGGAGCTGCT
CGCGATTGCTATTGCAGAGTTAGTGTCGCAAGGTTTGATCGATGAAGACAAATTGGATAC
CTTTAACGTGCCAAGCTATTGGCCATCTCTTGAAGAAGTGAAAGATATCGTCGAACGTGA
TGGCAGTTTCACTATTGACCATTTGGAGGGTTTCGAATTGGACTCTCTGGAGATGCAGGA
AAACGACAAATGGGTTCGCGGAGATAAATTTGCTAAGATGGTGCGGGCCTTTACTGAAC
CGATTATTTCAAACCAGTTCGGCCACGAGATTATGGACAAACTGTATGATAAATTTACCC
ACATTCTGGTTAGTGACCTTGAAGCAGAACTGCCGAAGACAACCAGCATTATTCTGGTAT
TGTCGAAGATAGTTGGGTAA
Polypeptide sequence encoded by the polynucleotide of SEQ ID NO: 71:
(SEQ ID NO: 72)
MGKVNEVLFMNRGEGEISYAQNSAFTQKVASMAMPALENAVETLFSKDFHLLQALTAADL
GCAAGPNTFAVISTIKRMMEKKCRELYCQTLELQVYLNDLFGNDFNTLFKGLSSEVVGNKCE
EVSCYVMGVPGSFHGRLFPRNSLHLVHSSYSVHWLTQAPKGLTSREGLALNKGKIYISKTSPP
VVKEAYLSQFHEDFTMFLNARSQEVVPNGCMVLILHGRQSSDPSEMESCFTWELLAIAIAEL
VSQGLIDEDKLDTFNVPSYWPSLEEVKDIVERDGSFTIDHLEGFELDSLEMQENDKWVRGDK
FAKMVRAFTEPIISNQFGHEIMDKLYDKFTHILVSDLEAELPKTTSIILVLSKIVG
DXMT_36:
(SEQ ID NO: 73)
ATGGAACTCGCTACTGCCGGTAAGGTTAATGAGGTTCTGTTTATGAACAGAGGCGAGGG
CGAATCTAGTTACGCACAAAACAGCTCATTTACACAGCAAGTCGCGAGCATGGCCCAGC
CCGCGCTCGAAAATGCAGTTGAAACGCTGTTTAGCAGAGATTTTCACTTACAAGCACTCA
ACGCTGCAGATCTGGGATGTGCTGCAGGCCCGAACACGTTTGCGGTAATCTCAACCATCA
AACGCATGATGGAGAAAAAGTGCCGGGAATTGAACTGTCAGACCCTGGAGTTACAGGTG
TACCTGAATGACCTGTTCGGCAATGACTTTAATACTCTGTTCAAGGGCCTCAGCAGTGAG
GTAATAGGTAATAAGTGCGAAGAAGTGAGCTGCTACGTAATGGGAGTGCCAGGTAGTTT
TCATGGACGGCTTTTCCCGCGCAATTCTCTTCACCTCGTTCACTCGAGCTATAGCGTTCAT
TGGTTAACACAAGCACCAAAGGGATTAACAAGCAGAGAGGGTTTAGCCTTGAACAAAGG
AAAAATCTACATCAGCAAAACTTCTCCGCCCGTAGTTAGAGAAGCGTACCTGTCACAATT
TCACGAGGACTTTACCATGTTCCTCAATGCGAGATCTCAGGAGGTGGTCCCCAACGGCTG
CATGGTTCTCATTCTGCGCGGGAGACAATGCTCAGATCCATCAGACATGCAATCTTGTTT
CACCTGGGAGCTGTTAGCCATGGCCATAGCCGAGCTCGTTTCGCAAGGCTTGATAGATGA
AGACAAGTTGGACACTTTTAATATACCCTCATATTTTGCTAGCCTGGAGGAGGTAAAGGA
CATTGTGGAGCGGGACGGCAGTTTTACAATCGACCATATAGAGGGGTTCGATCTCGATTC
ACTTGAGATGCAGGAGAACGACAAATGGGTCCGCGGCGAAAAATTTACTAAAGTAGTGC
GGGCCTTTACAGAACCCATTATCTCAAATCAGTTTGGACACGAAATCATGGATAAGCTGT
ATGATAAGTTCACTCACATCGTGGTCAGCGACTTGGAGGCGAAACTTCCCAAAACTACCA
GTATCTAA
Polypeptide sequence encoded by the polynucleotide of SEQ ID NO: 73:
(SEQ ID NO: 74)
MELATAGKVNEVLFMNRGEGESSYAQNSSFTQQVASMAQPALENAVETLFSRDFHLQALNA
ADLGCAAGPNTFAVISTIKRMMEKKCRELNCQTLELQVYLNDLFGNDFNTLFKGLSSEVIGN
KCEEVSCYVMGVPGSFHGRLFPRNSLHLVHSSYSVHWLTQAPKGLTSREGLALNKGKIYISK
TSPPVVREAYLSQFHEDFTMFLNARSQEVVPNGCMVLILRGRQCSDPSDMQSCFTWELLAM
AIAELVSQGLIDEDKLDTFNIPSYFASLEEVKDIVERDGSFTIDHIEGFDLDSLEMQENDKWVR
GEKFTKVVRAFTEPIISNQFGHEIMDKLYDKFTHIVVSDLEAKLPKTTSI
DXMT_37:
(SEQ ID NO: 75)
ATGAATACCGGTGAGGGTGAAAGCTCATATCTTCTTAACTCCAAATTTACCAACGTGACG
GCAATCAAGAGCATCCCTACCCTGAAGCGCGCCATTGAATCACTCTTTAAAGAGGAAAG
CCCGCCCTTTGAGCATTTACTGAACGTAGCGGATCTTGGATGTGCGTCTGGATCAACATC
GAATACCATTATGCCAACCGTAGTCCAGACGGTGGTTAATAAATGCCGTGAACTCAACC
ATAAAATTCCAGAATTCCAATTCTATCTGAATGATCTCCCTAGCAACGACTTTAATACTCT
GTTCAAGGGTCTGAACGGATTTGTGGGAAGCGGAGGAGAAGAGTTCGAGAATACGTCTT
GCCTTGTAATGGGTGCACCTGGCAGTTTTCATGGGCGCTTATTTCCACTCAACACGATAC
ATCTGGTATACTCAAATTATTCGGTTCACTGGTTGAGTAAAGTTCCCGATTTGCGCGACG
AAAAAGGTAACCCCATCAACAAAGGGACATTCTACATTAGCAAAACAAGCCCCAGTGGA
GTGCGTGAAGCTTATTTGGCACAATTTCAGAAAGATTTTACATTATTCTTGAAGTCCCGT
GCAGAAGAAATGGTTTCAAACGGTCGCGTAGTTCTCGTCTTGCACGGTCGTCTGAGTCAA
GACTTTAGCTGCGAAAAAGAGCTTCAACTGCCCTGGCTGATTTTGAGTCAAGCCATATCG
CGCCTGGTATCAAAAGGCTTGATCGACGAAGAGAAACTCGACTCATTTGAAGTACCGTA
CTATACTCCAAGCGTGCAGGAAGTGAAGGAATTGGTCGAAGGAGAAGGGTCGTACGCTG
TAGAGTTGATGGAAACTTTTACTATCCGTATCGGGGCTCGGAATGAAGGTATTTGGTCCG
ATGCAAGAGGTTTTGGAAATAACCTGCGTTCCATCACGGAAACGATGATAAGCCACCAC
TTCGGCCCGCAGATACTCGATGAGTTGTACGATGAGATTCAGGATTTACCTCTGCAGGAC
TTTGCAACACAGTGCTCATTCGTTGTCGGCTTAAAAAGAAATTAA
Polypeptide sequence encoded by the polynucleotide of SEQ ID NO: 75:
(SEQ ID NO: 76)
MNTGEGESSYLLNSKFTNVTAIKSIPTLKRAIESLFKEESPPFEHLLNVADLGCASGSTSNTIMP
TVVQTVVNKCRELNHKIPEFQFYLNDLPSNDFNTLFKGLNGFVGSGGEEFENTSCLVMGAPG
SFHGRLFPLNTIHLVYSNYSVHWLSKVPDLRDEKGNPINKGTFYISKTSPSGVREAYLAQFQK
DFTLFLKSRAEEMVSNGRVVLVLHGRLSQDFSCEKELQLPWLILSQAISRLVSKGLIDEEKLD
SFEVPYYTPSVQEVKELVEGEGSYAVELMETFTIRIGARNEGIWSDARGFGNNLRSITETMISH
HFGPQILDELYDEIQDLPLQDFATQCSFVVGLKRN
DXMT_38:
(SEQ ID NO: 77)
ATGGATATGAAAGACGTGCTTTGCATGAATACCGGTGAGGGCGAGAGTTCGTACTTGCT
GAATAGCAAGTTCACAAACGTAACAGCTATCAAATCTATCCCCACTCTTAAGAGAGCAA
TTGAATCGCTCTTCAAGGAAGAATCCCCTCCATTTGAACACTTATTGAATGTAGCCGATT
TAGGATGCGCCTCCGGCAGTACGAGTAATACGATAATGCCTACCGTGGTCCAAACGGTG
GTAAACAAATGTCGCGAACTGAACCACAAAATTCCTGAATTTCAATTCTACCTGAATGAC
TTGCCGTCAAATGACTTCAATACGTTGTTCAAGGGACTTAATGGATTTGTAGGTAGCGGT
GGTGAGGAATTTGAAAATACGAGTTGTCTCGTGATGGGTGCGCCCTAA
Polypeptide sequence encoded by the polynucleotide of SEQ ID NO: 77:
(SEQ ID NO: 78)
MDMKDVLCMNTGEGESSYLLNSKFTNVTAIKSIPTLKRAIESLFKEESPPFEHLLNVADLGCA
SGSTSNTIMPTVVQTVVNKCRELNHKIPEFQFYLNDLPSNDFNTLFKGLNGFVGSGGEEFENT
SCLVMGAP
DXMT_39:
(SEQ ID NO: 79)
ATGGAACTGGCAACCGCAGGAAAAGTAAATGAAGTACTCTTTATGAACCGCGGCGAAGG
AGAAAGCTCGTATGCCCAGAATTCAAGCTTTACACAGCAAGTTGCTTCTATGGCCCAGCC
AGCTCTTGAAAACGCTGTCGAAACTTTGTTCTCCCGGGATTTTCATCTCCAGGCTCTTAAC
GCTGCTGATTTGGGGTGCGCTGCAGGACCTAATACTTTCGCGGTCATCTCAACAATTAAA
CGCATGATGGAGAAGAAGTGTCGCGAACTCAACTGCCAGACGCTCGAACTCCAAGTCTA
CCTCAACGACCTCTTTGGGAACGACTTTAACACGCTGTTTAAAGGCTTGTCGAGTGAAGT
CATCGGAAATAAGTGTGAGGAGGTTAGTTGCTACGTGATGGGAGTACCGGGGTCATTCC
ACGGTCGTCTGTTTCCCAGAAACTCTCTGCATCTTGTCCATTCCTCATACTCAGTACATTG
GCTTACTCAGGCCCCAAAGGGCCTTACAAGCCGTGAAGGTCTGGCACTGAACAAGGGCA
AAATTTATATATCCAAGACTAGCCCTCCCGTTGTAAGAGAATCCTATTTATCCCAGTTCC
ATGAGGATTCCCAATGTTTTAGCATGTTAGACCCACGCGGGGGCTCACAGTGGTAA
Polypeptide sequence encoded by the polynucleotide of SEQ ID NO: 79:
(SEQ ID NO: 80)
MELATAGKVNEVLFMNRGEGESSYAQNSSFTQQVASMAQPALENAVETLFSRDFHLQALNA
ADLGCAAGPNTFAVISTIKRMMEKKCRELNCQTLELQVYLNDLFGNDFNTLFKGLSSEVIGN
KCEEVSCYVMGVPGSFHGRLFPRNSLHLVHSSYSVHWLTQAPKGLTSREGLALNKGKIYISK
TSPPVVRESYLSQFHEDSQCFSMLDPRGGSQW
DXMT_40:
(SEQ ID NO: 81)
ATGGAAGTGAAAGAGATGTTATTCATGAATAAAGGCGATGGCGAAAATTCATACGTGAA
AACATCGGGTTACACTCAGAAAGTGGCGGCCGTGACGCAACCCGTTGTATATCGGGCAG
CTCAGAGCCTGTTCACTGGTCGGAATTCTTGTAGTTATCAGGTTCTCAATGTCGCGGATTT
AGGGTGCTCAAGCGGACCTAATACCTTTACAGTGATGAGCACAGTGATTGAATCAACTC
GTGACAAATGCAGTGAGCTCAACTGGCAGATGCCAGAAATCCAGTTTTATCTGAATGATC
TGGTCGGTAACGATTTTAACACCTTGTTTAAAGGCCTGTCAGTTATACAGGATAAGTACA
AAAATGTAAGCTGTTTCGCCATGGGAGCTCCGGGCTCATTTCATGGACGTTTATTTCCCC
AAAACAGCATGCACCTCATACATAGCAGCTATGGGGTACAGTGGCTCTCTAAAGTACCG
AAAATGACCTCAGAAGGTGGTTTATCGCCGCCAAATAAAGGCAAAATTTATATCTCGAA
AACGTCTCCGCCCGCCGTGTGGAAAGCTTACCTCAGTCAATTTCAGGAGGACTTTTTAAG
TTTCTTGCGTTGTCGCAGTCCGGAGCTGGTTCCAGATGGACGCATGGTGCTCATTATTCAT
GGCCGTAAATCAGCTGACCCGACCACTAGAGAAAGCTGTTATACATGGGAAGTGCTTGC
AGATGCTATATCCTACCAGGTATCGCAAGGCTTAATTGACGAGGAAAAACTGAACTCCTT
CAATGTCCCTTACTATATTCCTAGTCAGGAAGAGGTACGGGATTTGGTTAATAAAGAAGG
CAGCTTCTTAACGGAGTTTGTAGACACAATCGAAGTAGAACTGGAGGGAATCTGGACTG
GCCCGGAAAACGGTGCCAAAAACCTGCGCAGCTTCACAGAACCAATGATTTCCCATCAG
TTCGGTGAGGAAGTCATGGACAAGCTGTATGACAAGGTCAAAGATATTCTGGTTGAGGA
CTGCAAACAAGAAAAACAATCTACGAGAGGAGTCTCTATAGTACTTGAGCTCAAAAAGA
AAGAGAGCCACCTTAGCTAA
Polypeptide sequence encoded by the polynucleotide of SEQ ID NO: 81:
(SEQ ID NO: 82)
MEVKEMLFMNKGDGENSYVKTSGYTQKVAAVTQPVVYRAAQSLFTGRNSCSYQVLNVAD
LGCSSGPNTFTVMSTVIESTRDKCSELNWQMPEIQFYLNDLVGNDFNTLFKGLSVIQDKYKN
VSCFAMGAPGSFHGRLFPQNSMHUHSSYGVQWLSKVPKMTSEGGLSPPNKGKIYISKTSPPA
VWKAYLSQFQEDFLSFLRCRSPELVPDGRMVLIIHGRKSADPTTRESCYTWEVLADAISYQVS
QGLIDEEKLNSFNVPYYIPSQEEVRDLVNKEGSFLTEFVDTIEVELEGIWTGPENGAKNLRSFT
EPMISHQFGEEVMDKLYDKVKDILVEDCKQEKQSTRGVSIVLELKKKESHLS
XMT_41:
(SEQ ID NO: 83)
ATGGAGCTCCAAGAAGTCCTGCGGATGAATGGAGGCGAAGGCGATACAAGCTACGCCAA
GAATTCAGCCTACAATCAACTGGTTCTCGCCAAGGTGAAACCTGTCCTTGAACAATGCGT
ACGGGAATTGTTGCGGGCCAACTTGCCCAACATCAACAAGTGCATTAAAGTTGCGGATTT
GGGATGCGCTTCTGGACCAAACACACTTTTAACGGTTCGGGACATTGTCCAAAGTATTGA
CAAAGTTGGCCAGGAAAAGAAGAATGAATTAGAACGTCCCACCATTCAGATTTTTCTGA
ATGATCTTTTCCCAAATGATTTCAATTCGGTTTTCAAGTTGCTGCCAAGCTTCTACCGCAA
ACTTGAGAAAGAAAATGGACGCAAAATAGGATCGTGCCTAATAGGGGCAATGCCCGGCT
CTTTCTACAGCAGACTCTTCCCCGAGGAGTCCATGCATTTTTTACACTCTTGTTACTGTCT
TCAATGGTTATCTCAGGTTCCTAGCGGTTTGGTGACTGAATTGGGGATCGGCACGAACAA
AGGGAGCATTTACTCTTCCAAAGCAAGTCGTCTGCCCGTCCAGAAGGCATATTTGGATCA
ATTTACGAAAGATTTTACCACATTTCTAAGGATTCATTCGGAAGAGTTGTTTTCACATGG
CCGAATGCTCCTTACTTGCATTTGTAAAGGAGTTGAATTAGACGCCCGGAATGCCATAGA
CTTACTTGAGATGGCAATAAACGACTTGGTTGTTGAGGGACATCTGGAGGAAGAAAAAT
TGGATAGTTTCAATCTTCCAGTCTATATACCTTCAGCAGAAGAAGTAAAGTGCATAGTTG
AGGAGGAAGGTTCTTTTGAAATTTTATACCTGGAGACTTTTAAGGTCCTTTACGATGCTG
GCTTCTCTATTGACGATGAACATATTAAAGCAGAGTATGTTGCATCTTCCGTTAGAGCAG
TTTACGAACCCATCCTCGCAAGTCATTTTGGAGAAGCTATTATACCTGACATATTCCACA
GGTTTGCGAAGCATGCAGCAAAGGTTCTCCCCTTGGGCAAAGGCTTCTATAATAATCTTA
TCATTTCTCTCGCCAAAAAGCCAGAGAAGTCAGACATGTAA
Polypeptide sequence encoded by the polynucleotide of SEQ ID NO: 83:
(SEQ ID NO: 84)
MELQEVLRMNGGEGDTSYAKNSAYNQLVLAKVKPVLEQCVRELLRANLPNINKCIKVADLG
CASGPNTLLTVRDIVQSIDKVGQEKKNELERPTIQIFLNDLFPNDFNSVFKLLPSFYRKLEKEN
GRKIGSCLIGAMPGSFYSRLFPEESMHFLHSCYCLQWLSQVPSGLVTELGIGTNKGSIYSSKAS
RLPVQKAYLDQFTKDFTTFLRIHSEELFSHGRMLLTCICKGVELDARNAIDLLEMAINDLVVE
GHLEEEKLDSFNLPVYIPSAEEVKCIVEEEGSFEILYLETFKVLYDAGFSIDDEHIKAEYVASSV
RAVYEPILASHFGEAIIPDIFHRFAKHAAKVLPLGKGFYNNLIISLAKKPEKSDM

### Example 1

### Guanine Deaminase (GDA) Gene Acquisition and Plasmid Construction

Nine genes with putative guanine deaminase activity were codon optimized and synthesized (SEQ ID NOS: 1, 3, 5, 7, 9, 11, 13, 15, and 17). Additionally, GDA genes were PCR amplified from both E. coli W3110 and E. coli BL-21 gDNA (SEQ ID NOS19 and 21). The nine synthetic and two PCR amplified (11 total) GDA genes were cloned into the pCK110900 vector system (See e.g., US Patent Application Publication 2006/0195947) under the control of a lac promoter. This expression vector also contains the P15a origin of replication and the chloramphenicol resistance gene. The resulting plasmids were transformed into E. coli W3110 using standard methods known in the art and the enzymes produced as described in Example 2.

### Example 2

### High-Throughput (HTP) Expression and Activity Assays of Guanine Deaminase (GDA) Variants

The putative guanine deaminase (GDA) polypeptides described in Example 1, were produced in host E. coli W3110 as an intracellular protein expressed under the control of the lac promoter. The polypeptides are designed to accumulate primarily as soluble cytosolic enzymes.

### High-Throughput Growth and Expression (HTP)

Single E. coli colonies of each variant were picked and grown for approximately 16-18 hours in LB media containing 1% glucose and 30 μg/mL chloramphenicol (CAM) under culture conditions of 30° C., 200 rpm, and 85% humidity. A 20 μL aliquot of this overnight growth was transferred to a deep well plate containing 380 μL Terrific Broth (TB) growth media containing 30 μg/mL CAM. The culture was incubated in a shaker for 2 hours at 30° C. and at 250 rpm to an OD₆₀₀ of about 0.6 to 0.8. The expression of the heterologous GDA genes was then induced with IPTG (1 mM final concentration). Incubation was continued for about 18 hours under the same conditions. After expression, cell cultures were centrifuged at 4000 rpm, 4° C. for 10 min., and the media discarded. Cell pellets were resuspended in 300 μL Lysis Buffer (20 mM Tris-HCl pH=7.5 containing 500 μg/mL polymyxin B sulfate (PMBS) and 500 μg/mL lysozyme) and shaken at room temperature for two hours. After lysis, cell debris was centrifuged at 4000 rpm, 4° C. for 10 min., and the resulting lysates were stored at 4° C.

### HTP Assay for GDA Activity

For each plate, 30 μL of HTP GDA lysates produced as describe above, were diluted into 1 mL of water. Twenty microliters of diluted lysates were then added to 180 μL of guanine solution (50 mM Tris-HCl pH=7.5, 30 μM guanine) at room temperature and absorbance measurements were immediately tracked at 245 nm and at 15 second intervals. GDA activity was determined based on the rate of depletion of the absorbance signal at 245 nm resulting from the conversion of guanine (with absorption peaks at about 245 and 270 nm) to xanthine (with a single absorption peak at about 270 nm only). After reacting for about 1 hr., the reactions were quenched with 50 μL of acetonitrile plus 0.2% formic acid, spun down at 4000 rpm for 5 min. and the supernatants were run on the HPLC to confirm the conversion of guanine to xanthine.

After one hour, GDA assay reactions with all nine GDA variants resulted in the complete conversion of guanine to xanthine as confirmed by HPLC analysis.

TABLE 2
Relative Activities¹ for Guanine Deaminase (GDA) Variants

| | | |
|---|---|---|
| GDA_01 | (SEQ ID NO: 2) | +++ |
| GDA_02 | (SEQ ID NO: 4) | +++ |
| GDA_03 | (SEQ ID NO: 6) | +++ |
| GDA_04 | (SEQ ID NO: 8) | +++ |
| GDA_05 | (SEQ ID NO: 10) | + |
| GDA_06 | (SEQ ID NO: 12) | +++ |
| GDA_07 | (SEQ ID NO: 14) | ++ |
| GDA_08 | (SEQ ID NO: 16) | ++ |
| GDA_09 | (SEQ ID NO: 18) | + |
| GDA_10 | (SEQ ID NO: 20) | +++ |
| GDA_11 | (SEQ ID NO: 22) | +++ |

¹Key:
+ = full conversion of 30 μM guanine to xanthine in >10 minutes
++ = full conversion of 30 μM guanine to xanthine in <10 minutes
+++ = full conversion of 30 μM guanine to xanthine in <3 minutes

### Example 3

### HPLC Analysis of Assay Samples

After running the high-throughput (HTP) or shake flask (SF) assays as described above, samples were quenched with acetonitrile (final concentration of acetonitrile was 20% v/v), shaken for minutes and spun to precipitate any particulates. The samples were then analyzed using an LC-UV assay by injecting 10 μL of the quenched reaction onto a 5 μL loop and resolved using the method described below.

Briefly, conversion to products of interest was determined using an Agilent 1200 HPLC equipped with a Phenomenex Luna C18 (2) column (4.6×250 mm, Sum). Solvents for LC analysis were 0.1% formic acid in water (A) and methanol (B). Products were resolved using the following gradient: t=0 min, 1% B; 7 min, 50% B; 7.75 min, 90% B; 8.0 min, 1% B, total run time is 10 min. The flow rate for the separation was 1.5 ml/min and a column temperature was ambient (approximately 21° C.). Retention times for key products: guanine=3.1 min; xanthine=4.5 min; 7-methyl-xanthine=5.4 min; 3-methyl-xanthine=5.8 min; 1-methyl-xanthine=6.2 min; theobromine=6.5 min; 1,7-dimethyl-xanthine=7.3 min; 1,3-dimethyl-xanthine=7.6 min; caffeine=8.3 min. Products in the eluant were determined as the peak area at 265 nm, with a path length of 1 cm.

### Example 4

### Methyltransferase (MT) Gene Acquisition and Plasmid Construction

Thirty-one genes with putative methyltransferase (MT) activities toward xanthine derivatives were codon optimized and synthesized as the gene sequences of SEQ ID NOS:23, 25, 27, 29, 31, 33, 35, 37, 39, 41, 43, 45, 47, 49, 51, 53, 55, 57, 59, 61, 63, 65, 67, 69, 71, 73, 65, 77, 79, and 81. Additionally, one gene with putative methyltransferase activity was synthesized with native sequence (not codon optimized; SEQ ID NO:83). These 32 synthetic MT genes were cloned into the pCK110900 vector system as described for the GDA genes in Example 1.

### Example 5

### High-Throughput (HTP) Expression and Activity Assay of Methyltransferase (MT) Enzymes on Caffeine Precursors

The putative methyltransferase (MT) polypeptides described in Example 4 were produced in host E. coli W3110 as an intracellular protein expressed under the control of the lac promoter. The polypeptides are designed to accumulate as soluble cytosolic enzymes.

### High-Throughput Growth and Expression (HTP)

Single E. coli colonies for each variant were picked and grown for approximately 16-18 hours (overnight) in LB media containing 1% glucose and 30 μg/mL chloramphenicol (CAM) under culture conditions of 30° C., 200 rpm, and 85% humidity. A 20 μL aliquot of overnight growth was transferred to a deep well plate containing 380 μL TB growth media containing 30 μg/mL CAM. The culture was incubated in a shaker for 2 hours at 30° C. and at 250 rpm to an OD₆₀₀ of about 0.6 to 0.8. The expression of the heterologous GDA genes was then induced with IPTG (IPTG) (1 mM final concentration). Incubation was continued for about 18 hours under the same conditions. After expression, cell cultures were centrifuged at 4000 rpm, 4° C. for 10 min., and the media discarded. Cell pellets were resuspended in 300 μL Lysis Buffer (20 mM Tris-HCl pH=7.5 containing 500 μg/mL PMBS and 500 μg/mL Lysozyme) and shaken at room temperature for two hours. After lysis, cell debris was centrifuged at 4000 rpm, 4° C. for 10 min., and the resulting lysates were stored at 4° C.

### HTP Assay for MT Activity

Xanthosine, xanthine, 7-methylxanthine, and theobromine were each diluted to about 400 μM in 50 mM Tris-HCl at pH=7.5. Twenty microliters of 10×S-adenosyl methionine (SAM) buffer (30 mM SAM, 50 mM Tris-HCl pH=7.5, 1 mM DTT) was added to each well on a 96-well plate along with 20 μl HTP lysate and 160 μl substrate. The final reaction plates (with about 320 μM substrate, 3 mM SAM, and 50 mM Tris-HCl pH=7.5) were sealed and incubated at 30° C. and 300 RPM for 24 hrs. in a Thermotron incubator. Reactions were quenched by adding 50 μl acetonitrile plus 0.2% formic acid to a final concentration of 20% acetonitrile. Reactions were spun down at 4000 RPM for 5 min. and the supernatants were analyzed on HPLC.

All 32 putative methyltransferase enzymes were tested for methyltransferase activity toward the natural caffeine precursors xanthosine, 7-methylxanthine, and theobromine, as well as the non-native precursor xanthine. Conversions for the major methylated products for each MT variant with each substrate are shown below:

TABLE 5-A
Relative Activity¹ of Each Methyltransferase Enzyme for
Methylating Xanthosine to the Indicated Products

| | | 3- | | | | 1,3-Me- |
|---|---|---|---|---|---|---|
| | | Me-X | 7-Me-X | Theo | Caff | X |
| MXMT_10 | (SEQ ID NO: 24) | ++ | + | + | −− | −− |
| MXMT_11 | (SEQ ID NO: 26) | −− | −− | −− | −− | −− |
| XMT_12 | (SEQ ID NO: 28) | −− | −− | −− | −− | −− |
| XMT_13 | (SEQ ID NO: 30) | −− | −− | −− | −− | −− |
| MXMT_14 | (SEQ ID NO: 32) | −− | −− | −− | −− | −− |
| DXMT_15 | (SEQ ID NO: 34) | −− | −− | −− | −− | −− |
| DXMT_16 | (SEQ ID NO: 36) | −− | −− | −− | −− | −− |
| DXMT_17 | (SEQ ID NO: 38) | −− | −− | −− | −− | −− |
| DXMT_18 | (SEQ ID NO: 40) | −− | −− | −− | −− | −− |
| DXMT_19 | (SEQ ID NO: 42) | −− | −− | −− | −− | −− |
| DXMT_20 | (SEQ ID NO: 44) | −− | −− | −− | −− | −− |
| DXMT_21 | (SEQ ID NO: 46) | −− | −− | −− | −− | −− |
| DXMT_22 | (SEQ ID NO: 48) | −− | −− | −− | −− | −− |
| DXMT_23 | (SEQ ID NO: 50) | −− | −− | −− | −− | −− |
| DXMT_25 | (SEQ ID NO: 52) | −− | −− | −− | −− | −− |
| DXMT_26 | (SEQ ID NO: 54) | −− | −− | −− | −− | −− |
| DXMT_27 | (SEQ ID NO: 56) | −− | −− | −− | −− | −− |
| DXMT_28 | (SEQ ID NO: 58) | −− | −− | −− | −− | −− |
| DXMT_29 | (SEQ ID NO: 60) | −− | +++ | −− | −− | −− |
| MXMT_30 | (SEQ ID NO: 62) | −− | −− | −− | −− | −− |
| MXMT_31 | (SEQ ID NO: 64) | −− | −− | −− | −− | −− |
| MXMT_32 | (SEQ ID NO: 66) | −− | −− | −− | −− | −− |
| MXMT_33 | (SEQ ID NO: 68) | + | −− | −− | −− | −− |
| MXMT_34 | (SEQ ID NO: 70) | + | −− | −− | −− | −− |
| MXMT_35 | (SEQ ID NO: 72) | + | −− | −− | −− | −− |
| DXMT_36 | (SEQ ID NO: 74) | −− | −− | −− | −− | −− |
| DXMT_37 | (SEQ ID NO: 76) | −− | −− | −− | −− | −− |
| DXMT_38 | (SEQ ID NO: 78) | −− | −− | −− | −− | −− |
| DXMT_39 | (SEQ ID NO: 80) | −− | −− | −− | −− | −− |
| DXMT_40 | (SEQ ID NO: 82) | −− | −− | −− | −− | −− |
| XMT_41 | (SEQ ID NO: 84) | −− | +++ | −− | −− | −− |

¹Key:
−− = conversion of <1% of substrate
+ = conversion of >1 to 10% of substrate
++ = conversion of >10 to 50% of substrate
+++ conversion of >50% of substrate

TABLE 5-B
Relative Activity¹ of Each Methyltransferase Enzyme for
Methylating Xanthine to the Indicated Products

| | | | | | | 1,3- |
|---|---|---|---|---|---|---|
| | | 3-Me-X | 7-Me-X | Theo | Caff | Me-X |
| MXMT_10 | (SEQ ID NO: 24) | +++ | + | + | −− | −− |
| MXMT_11 | (SEQ ID NO: 26) | ++ | −− | −− | −− | −− |
| XMT_12 | (SEQ ID NO: 28) | −− | −− | −− | −− | −− |
| XMT_13 | (SEQ ID NO: 30) | −− | −− | −− | −− | −− |
| MXMT_14 | (SEQ ID NO: 32) | −− | −− | −− | −− | −− |
| DXMT_15 | (SEQ ID NO: 34) | −− | −− | −− | −− | −− |
| DXMT_16 | (SEQ ID NO: 36) | −− | −− | −− | −− | −− |
| DXMT_17 | (SEQ ID NO: 38) | + | + | −− | + | ++ |
| DXMT_18 | (SEQ ID NO: 40) | −− | −− | −− | −− | −− |
| DXMT_19 | (SEQ ID NO: 42) | + | −− | −− | −− | −− |
| DXMT_20 | (SEQ ID NO: 44) | −− | −− | −− | −− | −− |
| DXMT_21 | (SEQ ID NO: 46) | −− | −− | −− | −− | −− |
| DXMT_22 | (SEQ ID NO: 48) | −− | −− | −− | −− | −− |
| DXMT_23 | (SEQ ID NO: 50) | −− | −− | −− | −− | −− |
| DXMT_25 | (SEQ ID NO: 52) | −− | −− | −− | −− | −− |
| DXMT_26 | (SEQ ID NO: 54) | −− | −− | −− | −− | −− |
| DXMT_27 | (SEQ ID NO: 56) | −− | −− | −− | −− | −− |
| DXMT_28 | (SEQ ID NO: 58) | −− | −− | −− | −− | −− |
| DXMT_29 | (SEQ ID NO: 60) | −− | −− | −− | −− | −− |
| MXMT_30 | (SEQ ID NO: 62) | −− | −− | −− | −− | −− |
| MXMT_31 | (SEQ ID NO: 64) | + | −− | −− | −− | −− |
| MXMT_32 | (SEQ ID NO: 66) | −− | −− | −− | −− | −− |
| MXMT_33 | (SEQ ID NO: 68) | ++ | −− | −− | −− | −− |
| MXMT_34 | (SEQ ID NO: 70) | + | −− | −− | −− | −− |
| MXMT_35 | (SEQ ID NO: 72) | +++ | −− | −− | −− | −− |
| DXMT_36 | (SEQ ID NO: 74) | −− | −− | −− | −− | −− |
| DXMT_37 | (SEQ ID NO: 76) | −− | −− | −− | −− | −− |
| DXMT_38 | (SEQ ID NO: 78) | −− | −− | −− | −− | −− |
| DXMT_39 | (SEQ ID NO: 80) | −− | −− | −− | −− | −− |
| DXMT_40 | (SEQ ID NO: 82) | −− | −− | −− | −− | −− |
| XMT_41 | (SEQ ID NO: 84) | −− | −− | −− | −− | −− |

¹Key:
−− = conversion of <1% of substrate
+ = conversion of >1 to 10% of substrate
++ = conversion of >10 to 50% of substrate
+++ conversion of >50% of substrate

TABLE 5-C
Relative Activity¹ of Each Methyltransferase Enzyme for
Methylating 7-Methylxanthine to the Indicated Products

| | | Theo | Caff |
|---|---|---|---|
| MXMT_10 | (SEQ ID NO: 24) | +++ | −− |
| MXMT_11 | (SEQ ID NO: 26) | +++ | −− |
| XMT_12 | (SEQ ID NO: 28) | −− | −− |
| XMT_13 | (SEQ ID NO: 30) | −− | −− |
| MXMT_14 | (SEQ ID NO: 32) | −− | −− |
| DXMT_15 | (SEQ ID NO: 34) | −− | −− |
| DXMT_16 | (SEQ ID NO: 36) | −− | −− |
| DXMT_17 | (SEQ ID NO: 38) | −− | +++ |
| DXMT_18 | (SEQ ID NO: 40) | −− | −− |
| DXMT_19 | (SEQ ID NO: 42) | ++ | ++ |
| DXMT_20 | (SEQ ID NO: 44) | −− | −− |
| DXMT_21 | (SEQ ID NO: 46) | −− | −− |
| DXMT_22 | (SEQ ID NO: 48) | −− | −− |
| DXMT_23 | (SEQ ID NO: 50) | −− | −− |
| DXMT_25 | (SEQ ID NO: 52) | −− | −− |
| DXMT_26 | (SEQ ID NO: 54) | −− | −− |
| DXMT_27 | (SEQ ID NO: 56) | −− | −− |
| DXMT_28 | (SEQ ID NO: 58) | −− | −− |
| DXMT_29 | (SEQ ID NO: 60) | + | −− |
| MXMT_30 | (SEQ ID NO: 62) | −− | −− |
| MXMT_31 | (SEQ ID NO: 64) | + | −− |
| MXMT_32 | (SEQ ID NO: 66) | −− | −− |
| MXMT_33 | (SEQ ID NO: 68) | ++ | −− |
| MXMT_34 | (SEQ ID NO: 70) | + | −− |
| MXMT_35 | (SEQ ID NO: 72) | +++ | −− |
| DXMT_36 | (SEQ ID NO: 74) | −− | −− |
| DXMT_37 | (SEQ ID NO: 76) | −− | −− |
| DXMT_38 | (SEQ ID NO: 78) | −− | −− |
| DXMT_39 | (SEQ ID NO: 80) | −− | −− |
| DXMT_40 | (SEQ ID NO: 82) | −− | −− |
| XMT_41 | (SEQ ID NO: 84) | −− | −− |

¹Key:
−− = conversion of <1% of substrate
+ = conversion of >1 to 10% of substrate
++ = conversion of >10 to 50% of substrate
+++ conversion of >50% of substrate

TABLE 5-D
Relative Activity¹ of Each Methyltransferase Enzyme
for Methylating Theobromine to Caffeine

| | | Caff |
|---|---|---|
| MXMT_10 | (SEQ ID NO: 24) | −− |
| MXMT_11 | (SEQ ID NO: 26) | −− |
| XMT_12 | (SEQ ID NO: 28) | −− |
| XMT_13 | (SEQ ID NO: 30) | −− |
| MXMT_14 | (SEQ ID NO: 32) | −− |
| DXMT_15 | (SEQ ID NO: 34) | −− |
| DXMT_16 | (SEQ ID NO: 36) | −− |
| DXMT_17 | (SEQ ID NO: 38) | +++ |
| DXMT_18 | (SEQ ID NO: 40) | + |
| DXMT_19 | (SEQ ID NO: 42) | +++ |
| DXMT_20 | (SEQ ID NO: 44) | −− |
| DXMT_21 | (SEQ ID NO: 46) | −− |
| DXMT_22 | (SEQ ID NO: 48) | −− |
| DXMT_23 | (SEQ ID NO: 50) | −− |
| DXMT_25 | (SEQ ID NO: 52) | −− |
| DXMT_26 | (SEQ ID NO: 54) | −− |
| DXMT_27 | (SEQ ID NO: 56) | −− |
| DXMT_28 | (SEQ ID NO: 58) | −− |
| DXMT_29 | (SEQ ID NO: 60) | −− |
| MXMT_30 | (SEQ ID NO: 62) | −− |
| MXMT_31 | (SEQ ID NO: 64) | −− |
| MXMT_32 | (SEQ ID NO: 66) | −− |
| MXMT_33 | (SEQ ID NO: 68) | −− |
| MXMT_34 | (SEQ ID NO: 70) | −− |
| MXMT_35 | (SEQ ID NO: 72) | −− |
| DXMT_36 | (SEQ ID NO: 74) | −− |
| DXMT_37 | (SEQ ID NO: 76) | −− |
| DXMT_38 | (SEQ ID NO: 78) | −− |
| DXMT_39 | (SEQ ID NO: 80) | −− |
| DXMT_40 | (SEQ ID NO: 82) | −− |
| XMT_41 | (SEQ ID NO: 84) | −− |

¹Key:
−− = conversion of <1% of substrate
+ = conversion of >1 to 10% of substrate
++ = conversion of >10 to 50% of substrate
+++ conversion of >50% of substrate

### Example 6

### Shake-Flask Expression and Activity Assay of Methyltransferase (MT) Enzymes with Xanthine as the Substrate

In addition to the HTP assay for primary screening, in some cases a secondary screening was carried out using shake-flask lysates of the methyltransferase variants. Shake flask lysates are prepared using mechanical lysis and are generally substantially more concentrated compared to the cell lysate used in HTP assays.

### Shake-Flask (SF) Expression

For preparing SF lysates, a single microbial colony of E. coli containing a plasmid encoding an methyltransferase of interest was inoculated into 5 mL Luria Bertani broth containing 30 μg/mL chloramphenicol (CAM) and 1% glucose. Cells were grown overnight (at least 16 hours) in an incubator at 30° C. with shaking at 250 rpm. The 5 mL culture was diluted into 250 mL of TB media containing 30 μg/ml CAM in a 1000 mL flask and was grown at 30° C. Expression of the methyltransferase genes were induced by addition of IPTG to a final concentration of 1 mM when the optical density at 600 nm (OD₆₀₀) of the culture was 0.6 to 0.8. Incubation was then continued overnight (at least 16 hours). Cells were harvested by centrifugation (7000 rpm, 6 min, 4° C.) and the supernatant discarded. The cell pellet was resuspended with 30 mL of cold (4° C.) 25 mM triethanolamine (TEA), pH 7.5 and passed once through a microfluidizer. Cell debris was removed by centrifugation (10000 rpm, 40 minutes, 4° C.). The clear lysate supernatant was collected and stored at 4° C. Alternatively, the clear lysate supernatant can be frozen at −80° C. and lyophilized to produce a dry shake-flask powder which is relatively stable when stored at −20° C.

### Assay for MT Activity on Xanthine Using Shake-Flask Lysates

Xanthine was diluted to about 400 μM in 50 mM Tris-HCl at pH=7.5. Twenty microliters of 10×S-Adenosyl methionine (SAM) buffer (30 mM SAM, 50 mM Tris-HCl pH=7.5, 1 mM DTT) was added to each well on a 96-well plate along with 50 μl HTP lysate and 130 μl substrate. The final reaction plates (with about 260 μM substrate, 3 mM SAM, and 50 mM Tris-HCl pH=7.5) were sealed and incubated at 30° C. and 300 RPM for 24 hrs. in a Thermotron incubator. Reactions were quenched by adding 50 μl acetonitrile plus 0.2% formic acid to a final concentration of 20% acetonitrile. Reactions were spun down at 4000 RPM for 5 min. and the supernatants were analyzed on HPLC.

Five MT variants (comprising SEQ ID NOS:24, 26, 38, 42, and 72) showing initial activity on xanthine in the HTP assay were scaled up and tested at shake-flask level. As shown in the table below, variants MXMT_10, MXMT_11, and MXMT_35 were shown to perform two methylations, converting xanthine to the dimethylated xanthine product theobromine (3,7-dimethylxanthine). Further, variants DXMT_17 and DXMT_19 were shown to perform three methylations, converting xanthine to the trimethylated xanthine product caffeine (1,3,7-trimethylxanthine). The table below shows some of the methylated products (as a percentage of initial xanthine peak area) from the methyltransferase reactions with each of the MT enzymes and xanthine as the substrate.

TABLE 6
Relative Activity¹ of Each Methyltransferase Enzyme for
Methylating Xanthine to the Indicated Products

| | | | | | | 1,3- |
|---|---|---|---|---|---|---|
| | | 3-Me-X | 7-Me-X | Theo | Caff | Me-X |
| MXMT_10 | (SEQ ID NO: 24) | ++ | ++ | ++ | −− | −− |
| MXMT_11 | (SEQ ID NO: 26) | +++ | ++ | + | −− | −− |
| DXMT_17 | (SEQ ID NO: 38) | + | ++ | −− | ++ | ++ |
| DXMT_19 | (SEQ ID NO: 42) | + | + | −− | + | ++ |
| MXMT_35 | (SEQ ID NO: 72) | +++ | ++ | −− | −− | −− |

¹ Key:
−− = conversion <1% of substrate
+ = conversion >1 to 10% of substrate
++ = conversion >10 to 50% of substrate
+++ = conversion >50% of substrate

### Example 7

### Conversion of 7-Methylxanthine to Theobromine and Caffeine with the Alternate Methyl Donor S-Methylmethionine (SMM)

In addition to the native methyl donor (SAM), five methyltransferase enzymes were tested for activity with the alternate methyl donor S-methylmethionine (SMM).

In this preliminary experiment, 7-Methylxanthine was diluted to about 400 μM in 50 mM Tris-HCl at pH=7.5. Twenty microliters of 10×S-methylmethionine (SMM) buffer (300 mM SMM, 50 mM Tris-HCl pH=7.5, 1 mM DTT) was added to each well on a 96-well plate along with 50 μl HTP lysate and 130 μl substrate. The final reaction plates (with about 260 μM substrate, 30 mM SMM, and 50 mM Tris-HCl pH=7.5) were sealed and incubated at 30° C. and 300 RPM for 24 hrs. in a Thermotron incubator. Reactions were quenched by adding 50 μl acetonitrile plus 0.2% formic acid to a final concentration of 20% acetonitrile. Reactions were spun down at 4000 RPM for 5 min. and the supernatants were analyzed on HPLC.

As shown in the table below, MXMT_10 (SEQ ID NO:24) was shown to methylate 7-methylxanthine in the presence of a composition containing the alternate methyl donor SMM to produce theobromine (3,7-dimethylxanthine). Further, DXMT_17 (SEQ ID NO:38) was shown to methylate 7-methylxanthine in the presence of a composition containing the alternate methyl donor SMM to produce the trimethylated product (1,3,7-trimethylxanthine). The table below summarized the primary methylated products (as a percentage of initial 7-methylxanthine peak area) from the methyltransferase reactions with the presence of a composition comprising the SMM methyl donor.

TABLE 7
Relative Activity¹ of Each Methyltransferase Enzyme for
Methylating 7-Methylxanthine to the Indicated Products with S-
Methylmethionine (SMM) Methyl Donor

| | | Theo | Caff |
|---|---|---|---|
| MXMT_10 | (SEQ ID NO: 24) | ++ | −− |
| MXMT_11 | (SEQ ID NO: 26) | −− | −− |
| DXMT_17 | (SEQ ID NO: 38) | ++ | + |
| DXMT_19 | (SEQ ID NO: 42) | −− | −− |
| MXMT_35 | (SEQ ID NO: 72) | −− | −− |

¹Key:
−− = conversion <1% of substrate
+ = conversion >1 to 10% of substrate
++ = conversion >10 to 50% of substrate
+++ = conversion >50% of substrate

### Example 8

### Conversion of 7-Methylxanthine to Theobromine and Caffeine with the Alternate Methyl Donor Gamma-Butyrobetaine

In addition to the native methyl donor (SAM), five methyltransferase enzymes were tested for activity with the alternate methyl donor gamma-butyrobetaine.

In this preliminary experiment, 7-Methylxanthine was diluted to about 400 μM in 50 mM Tris-HCl at pH=7.5. Twenty microliters of 10× gamma-butyrobetaine buffer (300 mM gamma-butyrobetaine, 50 mM Tris-HCl pH=7.5) was added to each well on a 96-well plate along with 50 μl HTP lysate and 130 μl substrate. The final reaction plates (with about 260 μM substrate, 30 mM gamma-butyrobetaine, and 50 mM Tris-HCl pH=7.5) were sealed and incubated at 30° C. and 300 RPM for 24 hrs. in a Thermotron incubator. Reactions were quenched by adding 50 μl acetonitrile plus 0.2% formic acid to a final concentration of 20% acetonitrile. Reactions were spun down at 4000 RPM for 5 min. and the supernatants were analyzed on HPLC.

As shown in the table below, DXMT_17 was shown to methylate 7-methylxanthine in the presence of a composition comprising the alternate methyl donor gamma-butyrobetaine to produce the trimethylated product (1,3,7-trimethylxanthine).

TABLE 8
Relative Activity¹ of Each Methyltransferase Enzyme for
Methylating 7-Methylxanthine to the Indicated Products with Gamma-
Butyrobetaine Methyl Donor

| | | Theo | Caff |
|---|---|---|---|
| MXMT_10 | (SEQ ID NO: 24) | −− | −− |
| MXMT_11 | (SEQ ID NO: 26) | −− | −− |
| DXMT_17 | (SEQ ID NO: 38) | ++ | + |
| DXMT_19 | (SEQ ID NO: 42) | −− | −− |
| MXMT_35 | (SEQ ID NO: 72) | −− | −− |

¹Key:
−− = conversion <1% of substrate
+ = conversion >1 to 10% of substrate
++ = conversion >10 to 50% of substrate
+++ = conversion >50% of substrate

### Example 9

### Conversion of Xanthine (or 7-Methylxanthine) to Theobromine and Caffeine with the Alternate Methyl Donor Trimethylglycine (TMG Aka Betaine)

In addition to the native methyl donor (SAM), methyltransferase enzymes are tested for activity with the alternate methyl donor trimethylglycine (TMG).

For these experiments, xanthine (or 7-methylxanthine) is diluted to about 400 μM in 50 mM Tris-HCl at pH=7.5. Twenty microliters of 10×TMG buffer (300 mM TMG, 50 mM Tris-HCl pH=7.5) is added to each well on a 96-well plate along with 50 μl HTP lysate and 130 μl substrate. The final reaction plates (with about 260 μM substrate, 30 mM TMG, and 50 mM Tris-HCl pH=7.5) are sealed and incubated at 30° C. and 300 RPM for 24 hrs. in a Thermotron incubator. Reactions are quenched by adding 50 μl acetonitrile plus 0.2% formic acid to a final concentration of 20% acetonitrile. Reactions are spun down at 4000 RPM for 5 min. and the supernatants are analyzed by HPLC.

While the present invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes in the United States of America, each and every publication and patent document cited in this application is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

1. A biosynthetic method for production of caffeine comprising: providing guanine, a guanine deaminase, at least one methyl transferase, and a methyl donor; contacting the guanine with the guanine deaminase to produce xanthine; contacting the xanthine with the methyl transferase and a methyl donor, under conditions wherein the xanthine is methylated, to produce a monomethylxanthine; contacting the monomethylxanthine with the methyl transferase and a methyl donor, under conditions wherein the monomethylxanthine is methylated, to produce a dimethylxanthine; and contacting the dimethylxanthine with the methyl transferase and a methyl donor, under conditions wherein the dimethylxanthine is methylated, to produce caffeine.

2. A biosynthetic method for production of caffeine comprising: providing guanine, a guanine deaminase, at least one methyl transferase, and a methyl donor; contacting said guanine with said guanine deaminase to produce xanthine; contacting said xanthine with said methyl transferase and a methyl donor, under conditions wherein said xanthine is methylated, to produce 7-methylxanthine; contacting said 7-methylxanthine with said methyl transferase and a methyl donor, under conditions wherein said 7-methylxanthine is methylated, to produce theobromine; and contacting said theobromine with said methyl transferase and a methyl donor, under conditions wherein said theobromine is methylated, to produce caffeine.

3. The biosynthetic method for production of caffeine of claim 1, wherein said methyl transferase is selected from XMT, MXMT, and DXMT.

4. The biosynthetic method for production of caffeine of claim 3, comprising at least two methyl transferases selected from XMT, MXMT, and/or DXMT.

5. The biosynthetic method for production of caffeine of claim 3, comprising the methyl transferases XMT, MXMT, and DXMT.

6. A biosynthetic method for the production of caffeine comprising: providing guanine, a guanine deaminase, at least one methyl transferase, and at least one methyl donor; contacting said guanine with said guanine deaminase to produce xanthine; contacting said xanthine with said methyl transferase and a methyl donor, under conditions wherein said xanthine is methylated to produce 7-methylxanthine; contacting said 7-methylxanthine with said methyl transferase and a methyl donor, under conditions wherein said 7-methylxanthine is methylated to produce theobromine; and contacting said theobromine with said methyl transferase and a methyl donor, under conditions wherein said theobromine is methylated to produce to produce caffeine.

7. The method of claim 1, wherein said guanine deaminase comprises a polypeptide selected from SEQ ID NOS:2, 4, 6, 8, 10, 12, 14, 16, 18, 20, and 22.

8. The method of claim 1, wherein said methyl transferase comprises a polypeptide selected from SEQ ID NOS:24, 26, 28, 30, 32, 34, 46, 38, 40, 42, 44, 46, 48, 50, 52, 54, 56, 58, 60, 62, 64, 66, 68, 70, 72, 74, 76, 78, 80, 82, or 84.

9. The method of claim 1, wherein said guanine deaminase is encoded by a polynucleotide selected from SEQ ID NOS:1, 3, 5, 7, 9, 11, 13, 15, 17, 19, and 21.

10. The method of claim 1, wherein said methyl transferase is encoded by a polynucleotide selected from SEQ ID NOS:3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, and/or 83.

11. The method of claim 1, wherein said methyl donor is SAM.

12. A non-naturally occurring polynucleotide sequence encoding a guanine deaminase, wherein said polynucleotide is codon-optimized and selected from SEQ ID NOS:2, 4, 6, 8, 10, 12, 14, 16, and 18.

13. A non-naturally occurring polynucleotide sequence encoding a methyl transferase, wherein said polynucleotide is codon-optimized and selected from SEQ ID NOS:24, 26, 28, 30, 32, 34, 46, 38, 40, 42, 44, 46, 48, 50, 52, 54, 56, 58, 60, 62, 64, 66, 68, 70, 72, 74, 76, 78, 80, and 82.

14. An expression vector comprising at least one polynucleotide sequence of claim 12.

15. An expression vector comprising at least one polynucleotide sequence of claim 13.

16. An expression vector comprising at least one polynucleotide sequence of claim 12.

17. A host cell comprising at least one expression vector of claim 14.

18. A method of expressing at least one non-naturally occurring polynucleotide, comprising placing the host cell of claim 17 under conditions suitable for the expression of said polynucleotide.
